(19) United States
(12) Patent Application Publication
(10) Pub. No.: US 2025/0205037 A1
(43) Pub. Date: Jun. 26, 2025

(54) STENTS WITH NON-CIRCULAR CROSS-SECTIONAL SHAPES

(71) Applicant: EDWARDS LIFESCIENCES CORPORATION, Irvine, CA (US)

(72) Inventors: Yuval Kasher, Kfar Shmuel (IL); Dikla Kersh, Karkur (IL); Roei Mayo, Irvine, CA (US); Emil Karapetian, Huntington Beach, CA (US); Ben Shitrit, Eilat (IL)

(73) Assignee: EDWARDS LIFESCIENCES CORPORATION, Irvine, CA (US)

(21) Appl. No.: 19/075,648

(22) Filed: Mar. 10, 2025

Related U.S. Application Data
(63) Continuation of application No. PCT/US2023/074853, filed on Sep. 22, 2023.
(60) Provisional application No. 63/377,187, filed on Sep. 26, 2022.

(51) Int. Cl. A61F 2/07

(52) U.S. Cl. CPC A61F 2/07; A61F 2210/0009; A61F 2210/0014; A61F 2220/0091; A61F 2230/0006; A61F 2230/0008; A61F 2230/001; A61F 2230/008; A61F 2250/0037; A61F 2250/0039

(57) ABSTRACT

An implant device includes a unitary stent frame including a first axial end segment having a circular cross-sectional shape, a second axial end segment having a circular cross-sectional shape, and a medial segment disposed between the first axial end segment and the second axial end segment, the medial segment having an oval cross-sectional shape. A fluid-tight covering can be disposed on minor-axis sidewall portions of the medial segment of the stent frame.

### RELATED APPLICATION(S)

This application is a continuation of International Patent Application No. PCT/US2023/074853, filed Sep. 22, 2023, which claims the benefit of U.S. Provisional Patent Application Ser. No. 63/377,187, filed on Sep. 26, 2022, the complete disclosures of which are hereby incorporated by reference in their entirety.

### BACKGROUND

The present disclosure generally relates to the field of medical implant devices. Insufficient or reduced compliance in certain blood vessels, including arteries such as the aorta, can result in reduced perfusion, cardiac output, and other health complications. Restoring compliance and/or otherwise controlling flow in such blood vessels can improve patient outcomes.

### SUMMARY

Described herein are devices, methods, and systems that facilitate the restoration of compliance characteristics to undesirably stiff blood vessels. Devices associated with the various examples of the present disclosure can include stents designed to cyclically reshape a target blood vessel segment in a manner as to affect blood flow therein. Such stents, and/or portions thereof, can be configured to transition between circular and non-circular (e.g., oval) shapes for the purpose of changing the volume of the target blood vessel segment. Such stents can have coverings, shapes, and/or other features that may promote efficient reshaping of the target blood vessel and/or hemostatic sealing between the stent and the blood vessel wall.

For purposes of summarizing the disclosure, certain aspects, advantages and novel features have been described. It is to be understood that not necessarily all such advantages may be achieved in accordance with any particular example. Thus, the disclosed examples may be carried out in a manner that achieves or optimizes one advantage or group of advantages as taught herein without necessarily achieving other advantages as may be taught or suggested herein.

Methods and structures disclosed herein for treating a patient also encompass analogous methods and structures performed on or placed on a simulated patient, which is useful, for example, for training; for demonstration; for procedure and/or device development; and the like. The simulated patient can be physical, virtual, or a combination of physical and virtual. A simulation can include a simulation of all or a portion of a patient, for example, an entire body, a portion of a body (e.g., thorax), a system (e.g., cardiovascular system), an organ (e.g., heart), or any combination thereof. Physical elements can be natural, including human or animal cadavers, or portions thereof; synthetic; or any combination of natural and synthetic. Virtual elements can be entirely in silica, or overlaid on one or more of the physical components. Virtual elements can be presented on any combination of screens, headsets, holographically, projected, loud speakers, headphones, pressure transducers, temperature transducers, or using any combination of suitable technologies.

Any of the various systems, devices, apparatuses, etc. in this disclosure can be sterilized (e.g., with heat, radiation, ethylene oxide, hydrogen peroxide, etc.) to ensure they are safe for use with patients, and the methods herein can comprise sterilization of the associated system, device, apparatus, etc. (e.g., with heat, radiation, ethylene oxide, hydrogen peroxide, etc.).

### BRIEF DESCRIPTION OF THE DRAWINGS

Various examples are depicted in the accompanying drawings for illustrative purposes and should in no way be interpreted as limiting the scope of the inventions. In addition, various features of different disclosed examples can be combined to form additional examples, which are part of this disclosure. Throughout the drawings, reference numbers may be reused to indicate correspondence between reference elements.

FIG. 1 illustrates example cardiac and vascular anatomy of a patient having a healthy, compliant aorta.

FIGS. 2A and 2B show side and axial cross-sectional views, respectively, of the healthy aorta of FIG. 1 experiencing compliant expansion.

FIG. 3 shows an example stiff aorta.

FIGS. 4A and 4B show side and axial cross-sectional views, respectively, of the stiff aorta of FIG. 3 experiencing compromised expansion.

FIGS. 5-1 and 5-2 show a blood vessel in circular and non-circular shapes, respectively.

FIGS. 6A-6D show perspective, side, and axial views, respectively, of a non-circular stent in accordance with one or more examples.

FIG. 7 shows an axial view of an uncovered, non-circular stent deployed within a blood vessel in accordance with one or more examples.

FIGS. 8A and 8B show perspective and axial views, respectively, of a non-circular stent having an outer covering in accordance with one or more examples.

FIGS. 9A and 9B show perspective and axial views, respectively, of a non-circular stent having an inner covering in accordance with one or more examples.

FIGS. 10A and 10B show perspective and axial views, respectively, of a non-circular stent having outer and inner coverings in accordance with one or more examples.

FIG. 11 shows a covered non-circular stent deployed within a blood vessel in accordance with one or more examples.

FIGS. 12A-12D show perspective, side, and axial views, respectively, of a stent having circular end portions and a non-circular medial portion in accordance with one or more examples.

FIGS. 13A and 13B show perspective and axial views, respectively, of a non-circular stent having inwardly-deflecting sidewalls in accordance with one or more examples.

FIG. 14 shows an axial view of an uncovered, non-circular stent deployed within a blood vessel in accordance with one or more examples.

FIG. 15 shows a non-circular stent experiencing buckling in accordance with one or more examples.

FIG. 16 shows a covered non-circular stent deployed within a blood vessel in accordance with one or more examples.

FIGS. 17A-17D show perspective, side, and axial views, respectively, of a stent having circular end portions and a non-circular medial portion in accordance with one or more examples.

FIGS. 18A-18D show perspective, side, and axial views, respectively, of a stent having oval (non-peanut) end portions and a peanut-shaped medial portion in accordance with one or more examples.

FIGS. 19-1, 19-2, and 19-3 illustrate a flow diagram for a process for reshaping a blood vessel using a non-circular stent in accordance with one or more examples.

FIGS. 20-1, 20-2, and 20-3 provide images of the stent and certain anatomy corresponding to operations of the process of FIGS. 19-1, 19-2, and 19-3 according to one or more examples.

FIG. 21 shows a covered non-circular stent having one or more fenestrations in accordance with one or more examples.

FIG. 22 shows an axial view of a non-circular stent having circumferential hinge features in accordance with one or more examples.

FIG. 23 shows a side view of a non-circular stent having circumferential hinge features in accordance with one or more examples.

FIGS. 24-1, 24-2, and 24-3 illustrate a flow diagram for a process for reshaping a blood vessel using a non-circular stent including hinge and stopper extension features in accordance with one or more examples.

FIGS. 25-1, 25-2, and 25-3 provide images of the stent and certain anatomy corresponding to operations of the process of FIGS. 24-1, 24-2, and 24-3 according to one or more examples.

FIGS. 26A-26D show perspective, side, and axial views, respectively, of a stent having circular end portions and a non-circular medial portion, wherein the end portions include scallop features in accordance with one or more examples.

FIG. 27 shows a stent with scalloped end portions implanted in a target blood vessel in accordance with one or more examples.

FIGS. 28A-28C show perspective, side, and axial views, respectively, of a stent having one or more balloons associated therewith in accordance with one or more examples.

FIG. 29 is a flow diagram illustrating a process for shaping a stent using one or more balloon devices in accordance with one or more examples.

FIGS. 30A-30C show perspective, side, and axial views, respectively, of a vessel-reshaping balloon device in accordance with one or more examples.

FIGS. 31A and 31C show perspective and axial views, respectively, of a stent-reshaping balloon reshaping a stent in accordance with one or more examples.

FIGS. 31B-1 and 31B-2 show the stent-reshaping balloon of FIGS. 31A and 31C in a blood vessel in circular and non-circular configurations, respectively, in accordance with one or more examples.

FIG. 32-1 shows a circular balloon disposed within a blood vessel in accordance with one or more examples.

FIGS. 32-2 and 32-3 show perspective and axial views, respectively, of a non-circular stent reshaping the balloon and blood vessel of FIG. 32-1 to a non-circular shape in accordance with one or more examples.

### DETAILED DESCRIPTION

The headings provided herein are for convenience only and do not necessarily affect the scope or meaning of the claimed invention.

Although certain preferred examples are disclosed below, it should be understood that the inventive subject matter extends beyond the specifically disclosed examples to other alternative examples and/or uses and to modifications and equivalents thereof. Thus, the scope of the claims that may arise herefrom is not limited by any of the particular examples described below. For example, in any method or process disclosed herein, the acts or operations of the method or process may be performed in any suitable sequence and are not necessarily limited to any particular disclosed sequence. Various operations may be described as multiple discrete operations in turn, in a manner that may be helpful in understanding certain examples; however, the order of description should not be construed to imply that these operations are order dependent. Additionally, the structures, systems, and/or devices described herein may be embodied as integrated components or as separate components. For purposes of comparing various examples, certain aspects and advantages of these examples are described. Not necessarily all such aspects or advantages are achieved by any particular example. Thus, for example, various examples may be carried out in a manner that achieves or optimizes one advantage or group of advantages as taught herein without necessarily achieving other aspects or advantages as may also be taught or suggested herein.

Certain reference numbers are re-used across different figures of the figure set of the present disclosure as a matter of convenience for devices, components, systems. features, and/or modules having features that may be similar in one or more respects. However, with respect to any of the examples disclosed herein, re-use of common reference numbers in the drawings does not necessarily indicate that such features, devices, components, or modules are identical or similar. Rather, one having ordinary skill in the art may be informed by context with respect to the degree to which usage of common reference numbers can imply similarity between referenced subject matter. Use of a particular reference number in the context of the description of a particular figure can be understood to relate to the identified device, component, aspect, feature, module, or system in that particular figure, and not necessarily to any devices, components, aspects, features, modules, or systems identified by the same reference number in another figure. Furthermore, aspects of separate figures identified with common reference numbers can be interpreted to share characteristics or to be entirely independent of one another.

Where an alphanumeric reference identifier is used that comprises a numeric portion and an alphabetic portion (e.g., ‘10a,’ ‘10’ is the numeric portion and ‘a’ is the alphabetic portion), references in the written description to only the numeric portion (e.g., ‘10’) may refer to any feature identified in the figures using such numeric portion (e.g., ‘10a,’ ‘10b,’ ‘10c,’ etc.), even where such features are identified with reference identifiers that concatenate the numeric portion thereof with one or more alphabetic characters (e.g., ‘a,’ ‘b,’ ‘c,’ etc.). That is, a reference in the present written description to a feature ‘10’ may be understood to refer to either an identified feature ‘10a’ in a particular figure of the present disclosure or to an identifier ‘10’ or ‘10b’ in the same figure or another figure, as an example.

Certain standard anatomical terms of location are used herein to refer to the anatomy of animals, and namely humans, with respect to various examples. Although certain spatially relative terms, such as “outer,” “inner,” “upper,” “lower,” “below,” “above,” “vertical,” “horizontal,” “top,” “bottom,” and similar terms, are used herein to describe a spatial relationship of one device/element or anatomical structure to another device/element or anatomical structure, it is understood that these terms are used herein for ease of description to describe the positional relationship between element(s)/structures(s), as illustrated in the drawings. It should be understood that spatially relative terms are intended to encompass different orientations of the element(s)/structures(s), in use or operation, in addition to the orientations depicted in the drawings. For example, an element/structure described as “above” another element/structure may represent a position that is below or beside such other element/structure with respect to alternate orientations of the subject patient or element/structure, and vice-versa. It should be understood that spatially relative terms. including those listed above, may be understood relative to a respective illustrated orientation of a referenced figure.

### Vascular Anatomy and Compliance

Certain examples are disclosed herein in the context of vascular implant devices, and in particular, compliance-enhancing stent implant devices implanted/implantable in the aorta. However, although certain principles disclosed herein may be particularly applicable to the anatomy of the aorta, it should be understood that compliance-enhancement implant devices in accordance with the present disclosure may be implanted in, or configured for implantation in, any suitable or desirable blood vessels or other anatomy, such as the inferior vena cava.

The anatomy of the heart and vascular system is described below to assist in the understanding of certain inventive concepts disclosed herein. In humans and other vertebrate animals, the heart generally comprises a muscular organ having four pumping chambers, wherein the flow thereof is at least partially controlled by various heart valves. namely, the aortic, mitral (or bicuspid), tricuspid, and pulmonary valves. The valves may be configured to open and close in response to a pressure gradient present during various stages of the cardiac cycle (e.g., relaxation and contraction) to at least partially control the flow of blood to a respective region of the heart and/or to blood vessels (e.g., ventricles, pulmonary artery, aorta, etc.). The contraction of the various heart muscles may be prompted by signals generated by the electrical system of the heart.

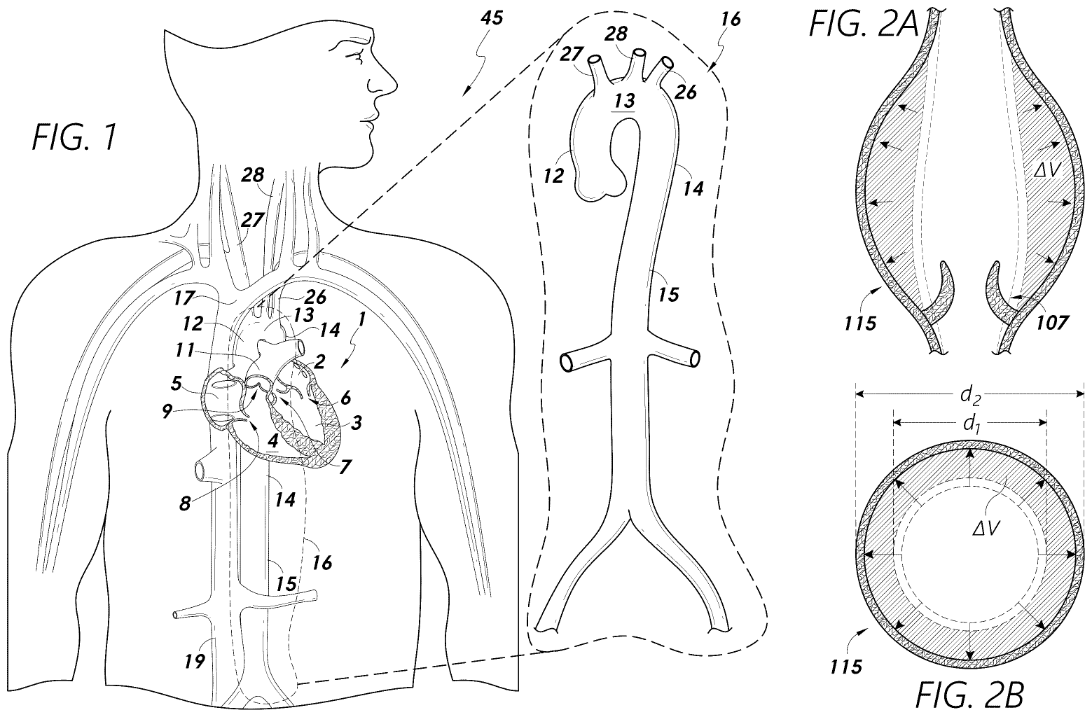

FIG. 1 illustrates an example representation of a heart 1 and associated vasculature having various features relevant to one or more examples of the present inventive disclosure. The heart 1 includes four chambers, namely the left atrium 2, the left ventricle 3, the right ventricle 4, and the right atrium 5. In terms of blood flow, blood generally flows from the right ventricle 4 into the pulmonary artery via the pulmonary valve 9, which separates the right ventricle 4 from the pulmonary artery 11 and is configured to open during systole so that blood may be pumped toward the lungs and close during diastole to prevent blood from leaking back into the heart from the pulmonary artery 11. The pulmonary artery 11 carries deoxygenated blood from the right side of the heart to the lungs. The pulmonary artery 11 includes a pulmonary trunk and left and right pulmonary arteries that branch off of the pulmonary trunk, as shown.

The tricuspid valve 8 separates the right atrium 5 from the right ventricle 4. The tricuspid valve 8 generally has three cusps/leaflets and may generally close during ventricular contraction (i.e., systole) and open during ventricular expansion (i.e., diastole). The mitral valve 6 generally has two cusps/leaflets and separates the left atrium 2 from the left ventricle 3. The mitral valve 6 is configured to open during diastole so that blood in the left atrium 2 can flow into the left ventricle 3, and, when functioning properly, closes during systole to prevent blood from leaking back into the left atrium 2. The aortic valve 7 separates the left ventricle 3 from the aorta 12. The aortic valve 7 is configured to open during systole to allow blood leaving the left ventricle 3 to enter the aorta 12, and close during diastole to prevent blood from leaking back into the left ventricle 3.

The heart valves may generally comprise a relatively dense fibrous ring, referred to herein as the annulus, as well as a plurality of leaflets or cusps attached to the annulus. Generally, the size of the leaflets or cusps may be such that when the heart contracts the resulting increased blood pressure produced within the corresponding heart chamber forces the leaflets at least partially open to allow flow from the heart chamber. As the pressure in the heart chamber subsides, the pressure in the subsequent chamber or blood vessel may become dominant and press back against the leaflets. As a result, the leaflets/cusps come in apposition to each other, thereby closing the flow passage. Dysfunction of a heart valve and/or associated leaflets (e.g., pulmonary valve dysfunction) can result in valve leakage and/or other health complications.

The atrioventricular (mitral and tricuspid) heart valves generally are coupled to a collection of chordae tendineae and papillary muscles (not shown for visual clarity) for securing the leaflets of the respective valves to promote and/or facilitate proper coaptation of the valve leaflets and prevent prolapse thereof. The papillary muscles, for example, may generally comprise finger-like projections from the ventricle wall. The valve leaflets are connected to the papillary muscles by the chordae tendineae. A wall of muscle 17. referred to as the septum, separates the left 2 and right 5 atria and the left 3 and right 4 ventricles.

The vasculature of the human body, which may be referred to as the circulatory system, cardiovascular system, or vascular system, contains a complex network of blood vessels with various structures and functions and includes various veins (venous system) and arteries (arterial system). Generally, arteries, such as the aorta 16, carry blood away from the heart, whereas veins, such as the inferior and superior venae cavae, carry blood back to the heart.

The aorta 16 is a compliant arterial blood vessel that buffers and conducts pulsatile left ventricular output and contributes the largest component of total compliance of the arterial tree. The aorta 16 includes the ascending aorta 12, which begins at the opening of the aortic valve 7 in the left ventricle of the heart. The ascending aorta 12 and pulmonary trunk 11 twist around each other, causing the aorta 12 to start out posterior to the pulmonary trunk 11, but end by twisting to its right and anterior side. Among the various segments of the aorta 16, the ascending aorta 12 is relatively more frequently affected by aneurysms and dissections, often requiring open heart surgery to be repaired. The transition from ascending aorta 12 to aortic arch 13 is at the pericardial reflection on the aorta. At the root of the ascending aorta 12, the lumen has three small pockets between the cusps of the aortic valve and the wall of the aorta, which are called the aortic sinuses or the sinuses of Valsalva. The left aortic sinus contains the origin of the left coronary artery and the right aortic sinus likewise gives rise to the right coronary artery. Together, these two arteries supply the heart.

As mentioned above, the aorta is coupled to the heart 1 via the aortic valve 7, which leads into the ascending aorta 12 and gives rise to the innominate artery 27, the left common carotid artery 28, and the left subclavian artery 26 along the aortic arch 13 before continuing as the descending thoracic aorta 14 and further the abdominal aorta 15. References herein to the aorta may be understood to refer to the ascending aorta 12 (also referred to as the “ascending thoracic aorta”), aortic arch 13, descending or thoracic aorta 14 (also referred to as the “descending thoracic aorta”), abdominal aorta 15, or other arterial (or venous) blood vessel or portion thereof.

Arteries, such as the aorta 16, may utilize blood vessel compliance (e.g., arterial compliance) to store and release energy through the stretching of blood vessel walls. The term “compliance” is used herein according to its broad and ordinary meaning, and may refer to the ability of an arterial blood vessel or prosthetic implant device to distend, expand, stretch, or otherwise deform in a manner as to increase in volume in response to increasing transmural pressure, and/or the tendency of a blood vessel (e.g., artery) or prosthetic implant device, or portion thereof, to recoil toward its original dimensions as transmural pressure decreases.

Arterial compliance facilitates perfusion of organs in the body with oxygenated blood from the heart. Generally, a healthy aorta and other major arteries in the body are at least partially elastic and compliant, such that they can act as a reservoir for blood, filling up with blood when the heart contracts during systole and continuing to generate pressure and push blood to the organs of the body during diastole. In older individuals and patients suffering from heart failure and/or atherosclerosis, compliance of the aorta and other arteries can be diminished to some degree or lost. Such reduction in compliance can reduce the supply of blood to the organs of the body due to the decrease in blood flow during diastole. Among the risks associated with insufficient arterial compliance, a significant risk presented in such patients is a reduction in blood supply to the heart muscle itself. For example, during systole, generally little or no blood may flow in the coronary arteries and into the heart muscle due to the contraction of the heart which holds the heart at relatively high pressures. During diastole, the heart muscle generally relaxes and allows flow into the coronary arteries. Therefore, perfusion of the heart muscle relies on diastolic flow, and therefore on aortic/arterial compliance.

Insufficient perfusion of the heart muscle can lead to and/or be associated with heart failure. Heart failure is a clinical syndrome characterized by certain symptoms. including breathlessness, ankle swelling, fatigue, and others. Heart failure may be accompanied by certain signs, including elevated jugular venous pressure, pulmonary crackles and peripheral edema, for example, which may be caused by structural and/or functional cardiac abnormality. Such conditions can result in reduced cardiac output and/or elevated intra-cardiac pressures at rest or during stress.

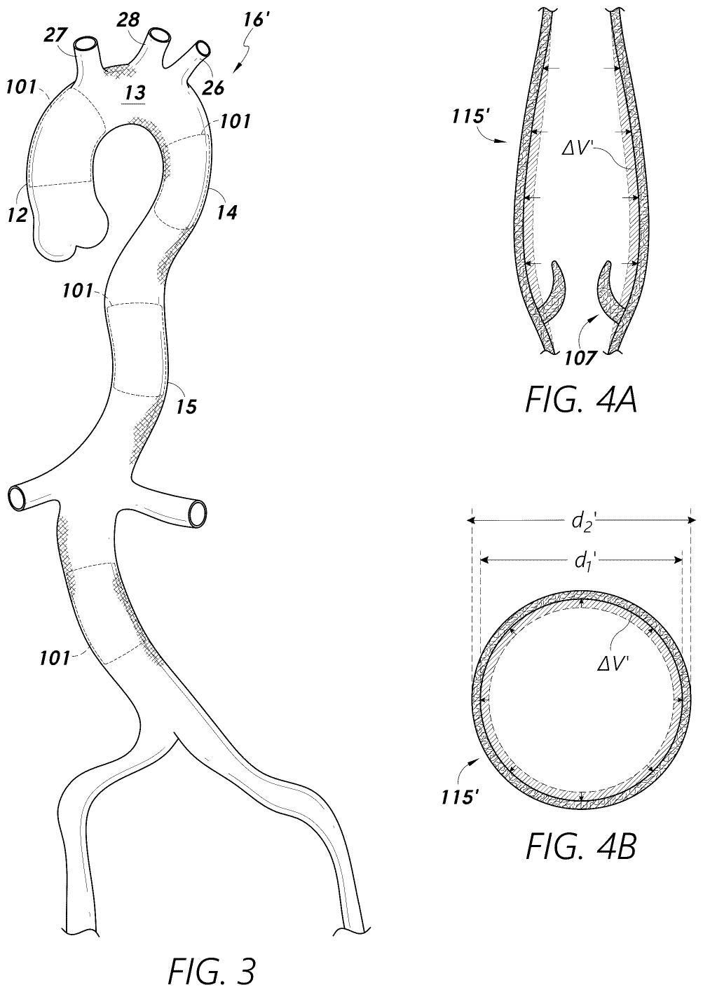

FIGS. 2A and 2B show side and axial cross-sectional views, respectively, of the healthy aorta 16 of FIG. 1 experiencing compliant expansion and contraction over a cardiac cycle. FIG. 3 shows an example stiff aorta 16′, whereas FIGS. 4A and 4B show side and axial cross-sectional views, respectively, of the stiff aorta 16′ of FIG. 3 experiencing compromised expansion and contraction over a cardiac cycle.

The systolic phase of the cardiac cycle is associated with the pumping phase of the left ventricle, while the diastolic phase of the cardiac cycle is associated with the resting or filling phase of the left ventricle. As shown in FIGS. 2A and 2B, with proper arterial compliance, an increase in volume 4v will generally occur in an artery when the pressure in the artery is increased from diastole to systole. With respect to the aorta, as blood is pumped into the aorta 115 through the aortic valve 107, the pressure in the aorta increases and the diameter of at least a portion of the aorta expands. A first portion of the blood entering the aorta 115 during systole may pass through the aorta during the systolic phase, while a second portion (e.g., approximately half of the total blood volume) may be stored in the expanded volume Av caused by compliant stretching of the blood vessel 115, thereby storing energy for contributing to perfusion during the diastolic phase. A compliant aorta may generally stretch with each heartbeat, such that the diameter of at least a portion of the aorta expands.

The tendency of the arteries to stretch in response to pressure as a result of arterial compliance may have a significant effect on perfusion and/or blood pressure in some patients. For example, arteries with relatively higher compliance may be conditioned to more easily deform than lower-compliance arteries under the same pressure conditions. Compliance (C) may be calculated using the following equation, where Δv is the change in volume (e.g., in mL) of the blood vessel, and Δp is the pulse pressure from systole to diastole (e.g., in mmHg):

$$C = \frac{Δ v}{Δ p} \qquad ( 1 )$$

Aortic stiffness and reduced compliance can lead to elevated systolic blood pressure, which can in turn lead to elevated intracardiac pressures, increased afterload, and/or other complications that can exacerbate heart failure. Aortic stiffness further can lead to reduced diastolic flow, which can lead to reduced coronary perfusion, decreased cardiac supply, and/or other complications that can likewise exacerbate heart failure.

Healthy arterial compliance may cause retraction/recoil of the blood vessel wall inward during diastole, thereby creating pressure in the blood vessel to cause blood to continue to be pushed through the artery 115 when the valve 107 is closed. For example, during systole, approximately 50% of the blood that enters the artery 115 through the valve 107 may be passed through the artery, whereas the remaining 50% may be stored in the artery, as enabled by expansion of the vessel wall. Some or all of the stored portion of blood in the artery 115 may be pushed through the artery by the contracting vessel wall during diastole. For patients experiencing arterial stiffness that causes lack of compliance, their arteries may not operate effectively in accordance with the expansion/contraction functionality shown in FIGS. 2A and 2B.

As shown in FIG. 3, the aorta tends to change in shape as a function of age, resulting in a higher degree of curvature and/or tortuosity over time. As the vasculature of a subject becomes less elastic, arterial blood pressure (e.g., left-ventricular afterload) becomes more pulsatile, which can have a deleterious effect, such as the thickening of the left ventricle muscle and/or diastolic heart failure. Stiffness in the aorta and/or other blood vessel(s) can occur due to an increase in collagen content and/or a corresponding decrease in elastin. While stiff/non-compliant blood vessels can generally suffer from a lack of elasticity in the walls thereof, as shown as causing compromised/reduced stretching and volume change Δv′, such vessels can maintain some amount of flexibility/bendability, such that reshaping of the blood vessels can occur without necessarily requiring the stretching of the walls of the blood vessel.

Generally, the majority of aortic compliance is provided in the ascending aorta 12 with respect to healthy anatomy. Furthermore, calcification frequently occurs in the area of the ascending aorta 12, near the aortic arch 13 and the great vessels emanating therefrom. Such anatomical areas can experience relatively higher stresses due to the geometry, elasticity, and flow dynamics associated therewith. Therefore, implantation/deployment of compliance-enhancing, non-circular stent implant devices of the present disclosure can advantageously be in the ascending aorta 12 in some cases. While relatively less calcification tends to occur in the descending 14 and abdominal 15 aorta. implant devices of the present disclosure can advantageously be implanted/deployed in such areas as well for the purpose of increasing compliance in the aortic system. Examples of the present disclosure provide compliance-enhancing stent implant devices, which may be implanted in one or more locations in a compromised aorta and/or other vessel(s). For example, FIG. 3 shows example positions of stent devices 101 including features disclosed herein implanted in various areas of an aorta 16′.

### Compliance-Enhancing Stent Implants

The present disclosure relates to systems, devices, and methods for adding-back and/or increasing compliance in the aorta or other arterial (or venous) blood vessel(s) to provide improved perfusion of the heart muscle and/or other organ(s) of the body. Examples of the present disclosure can include stents that, when implanted, are configured to decrease the cross-sectional area/volume of the blood vessel segment in which the stent is implanted during low-pressure conditions, such as diastole, which serves to force blood through the blood vessel segment by pushing the blood through the vessel as the vessel volume reduces in connection with stent contraction induced by cyclical drops in blood pressure.

The non-circular (e.g., oval- and/or peanut-shaped) stents of the present disclosure can advantageously be configured to generate a differential cross-sectional area or volume of the target blood vessel(s) (e.g., aorta) between high- and low-pressure phases of the cardiac cycle to facilitate perfusion. As described above, relatively non-compliant blood vessels generally may not be able to stretch to thereby lengthen the perimeter of the blood vessel in response to increased pressure conditions. Such inability to stretch can prevent compliant expansion of the blood vessel.

As the stents of the present disclosure produce complaint blood vessel volume change by manipulating/reshaping the native blood vessel walls, compliance can be increased in the target blood vessel without requiring blood vessel grafting or resection. Therefore, compared to blood flow solutions involving blood vessel grafting/resection, examples of the present disclosure can provide a solution that avoids the risks that may be associated with cutting of the vessel and/or devices grafted in/to such vessels, which may present risk of rupture and blood leakage outside of the circulatory system. Hazards associated with extravascular arterial blood leakage, such as within the abdominal and/or chest cavity, can include the risk of serious injury or death.

As described above, desirable diastolic flow in arterial (or venous) blood vessels is enabled by the decrease in cross-sectional area/volume of the blood vessels when transitioning from higher-pressure conditions (e.g., systole) to lower-pressure conditions (e.g., diastole). Where the relevant blood vessel has become stiff and non-compliant, stretching/expanding and subsequent contraction/shrinking of the blood vessel to cause the desired change in area/volume of the blood vessel may be limited due to the perimeter/wall of the blood vessel being resistant to stretching. Examples of the present disclosure provide implants that cause a change in cross-sectional area/volume of a target blood vessel without requiring stretching in the blood vessel wall. Rather, such cyclical change in blood vessel area/volume can be achieved through manipulation of the shape (e.g., cross-sectional shape) of the target blood vessel, wherein a transition between blood vessel shapes occurring in response to changing pressure conditions can reduce and increase the area/volume of the blood vessel in a cyclical manner to promote more even flow of blood through the blood vessel throughout the cardiac cycle.

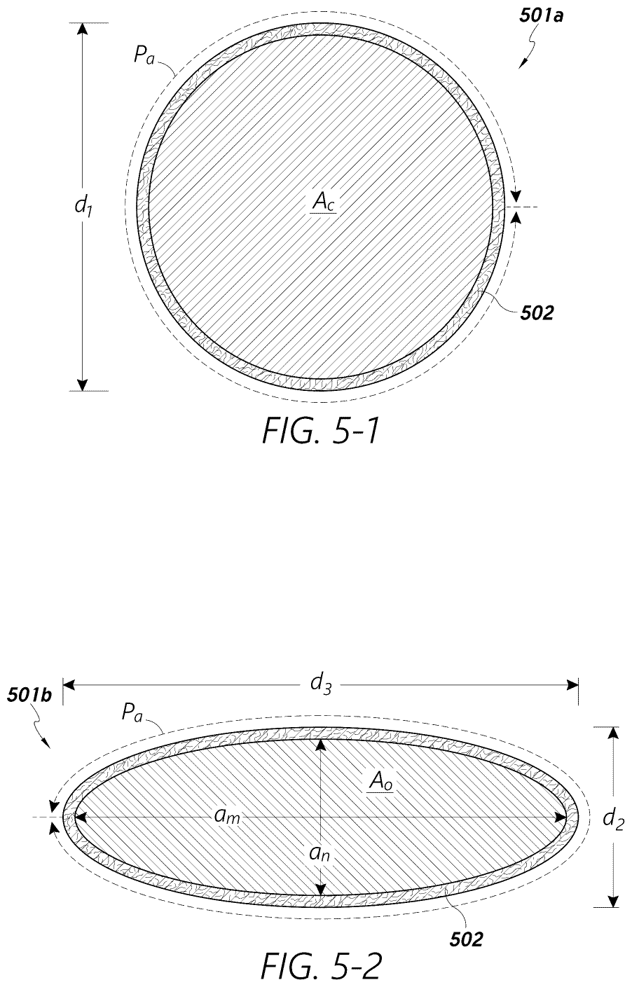

With respect to a blood vessel having a relatively fixed perimeter, wherein the blood vessel wall does not expand sufficiently due to stiffness and/or other factors of non-compliance, generally, the greatest area/volume of the blood vessel may be present/achieved when the blood vessel wall forms a circular cross-sectional shape, which may maximize the cross-sectional area and volume of the blood vessel. FIG. 5-1 shows an example blood vessel 501 (identified as blood vessel 501a in FIG. 5-1) having a generally circular cross-sectional shape, such that the area A_{c} thereof is maximized for the given perimeter/wall-length Pa. In the circular configuration, the diameter di is substantially constant at every angle about the axis of the vessel.

Diverging from a circular cross-sectional shape can produce a cross-sectional area/volume for a blood vessel that is less than the maximum area A_{c} shown in FIG. 5-1. For example, FIG. 5-2 shows the blood vessel 501 (identified as vessel 501b in FIG. 5-2) having a shape that resembles an oval/ellipse, which produces the cross-sectional area Aₒ that is less than the area A_{c} with the same blood vessel wall/perimeter length Pₐ. The oval shape of the vessel 501b may have a major axis aₘ having a dimension d₃ that is greater than a dimension d₂ of the minor axis aₙ thereof.

With further reference to FIGS. 5-1 and 5-2, due to the area Aₒ of the oval vessel of FIG. 5-1 being less than the area A_{c} of the circular configuration shown in FIG. 5-1, transitioning from the circular shape 501a to the non-circular shape 501b, can provide a reduction in area/volume of the blood vessel, and therefore solutions that cause transitions between circular and non-circular blood vessel shapes between cardiac phases can provide compliance characteristics without the need for elasticity in the blood vessel wall tissue. For example, where a mechanism is implemented to cause a blood vessel to transition between circular and non-circular shapes in response to changing pressure conditions, such manipulation of the blood vessel shape can introduce volumetric change in the blood vessel in response to the typical changes in pressure experienced during the cardiac cycle, thereby increasing cardiac efficiency and reducing pulsatile load.

In view of the foregoing, examples of the present disclosure provide stent implant devices and associated processes configured to transition the shape/area of a blood vessel from circular/more-circular to non-circular/less-circular shapes, and vice versa, to enhance compliance with respect to the area of the implant reshaping. Such stent implant devices/processes may affect vessel reshaping through dynamic reshaping of the structural shape of the stent in a way that produces a change in shape of the blood vessel in which it is implanted to produce a change in blood vessel area/volume between the systolic and diastolic phases of the cardiac cycle. As described above, for relatively stiff blood vessels, radial outward expansion/stretching of the blood vessel sufficient to achieve a change in volume that produces desirable compliance may not occur as pressure conditions change. Using stent implant devices in accordance with aspects of the present disclosure may be desirable to provide the necessary change in volume of the target blood vessel.

Examples of the present disclosure provide for stent-type implants that are biased to a non-circular cross-sectional area, such that, in a relaxed/non-pressurized state, a first diameter of the stent has a greater dimension along a major axis compared to a second diameter of the stent along a minor axis, wherein such stents are configured to transition to a more-circular shape when pressure within the blood vessel overcomes the non-circular bias of the stent and causes the stent walls to be pushed to the more-circular configuration. The ability of stent implant devices of the present disclosure to reshape the target blood vessel in the manner described above to produce the desired oval cross-section of the blood vessel can be achievable due to stiff/non-compliant blood vessels, which may be unable to stretch to a substantial degree, still retaining the ability to bend to a sufficient degree to allow for such shaping of the blood vessel. That is, the bending stiffness of a non-compliant blood vessel may be relatively lower compared to the stretching stiffness thereof. Therefore, examples of the present disclosure achieve compliance through bending energy with respect to the blood vessel wall, as opposed to stretching energy. When stents of the present disclosure are forced to a circular, or relatively more-circular, axial cross-sectional shape, energy may be stored in the shape memory of the walls of the stent, wherein recoil/contraction of the stent towards its biased, oval/non-circular configuration can return/release energy to the blood circulation.

In some examples, a stent and/or similar device may provide an added compliance (i.e., added change in volume over a constant change in pressure) to any blood vessel in or on which it is placed. The term “stent” is used herein in accordance with its broad and ordinary meaning and may refer to any device configured to be implanted in a lumen of a blood vessel, the device having a tubular form forming a lumen through which blood can flow.

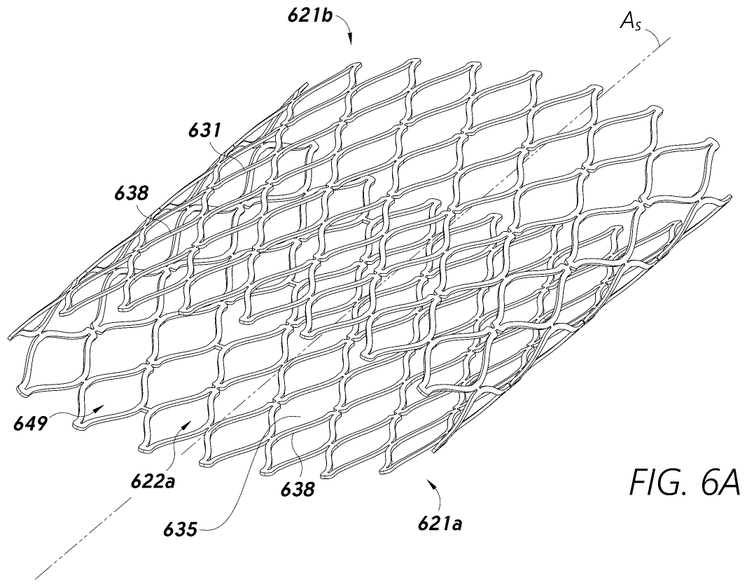

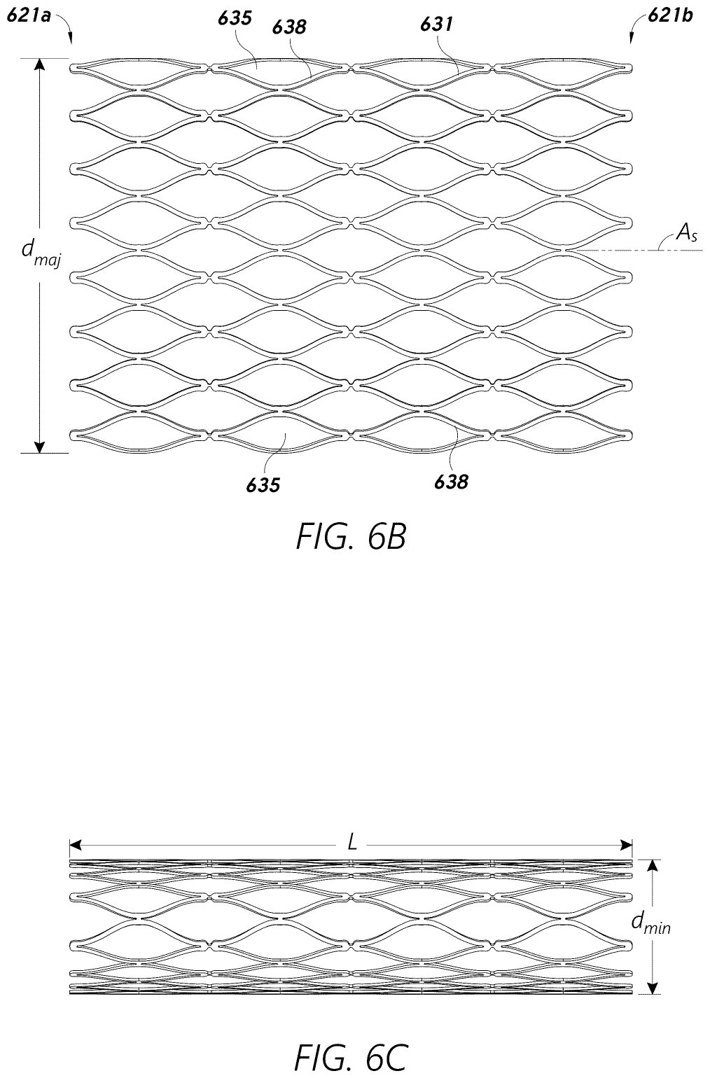

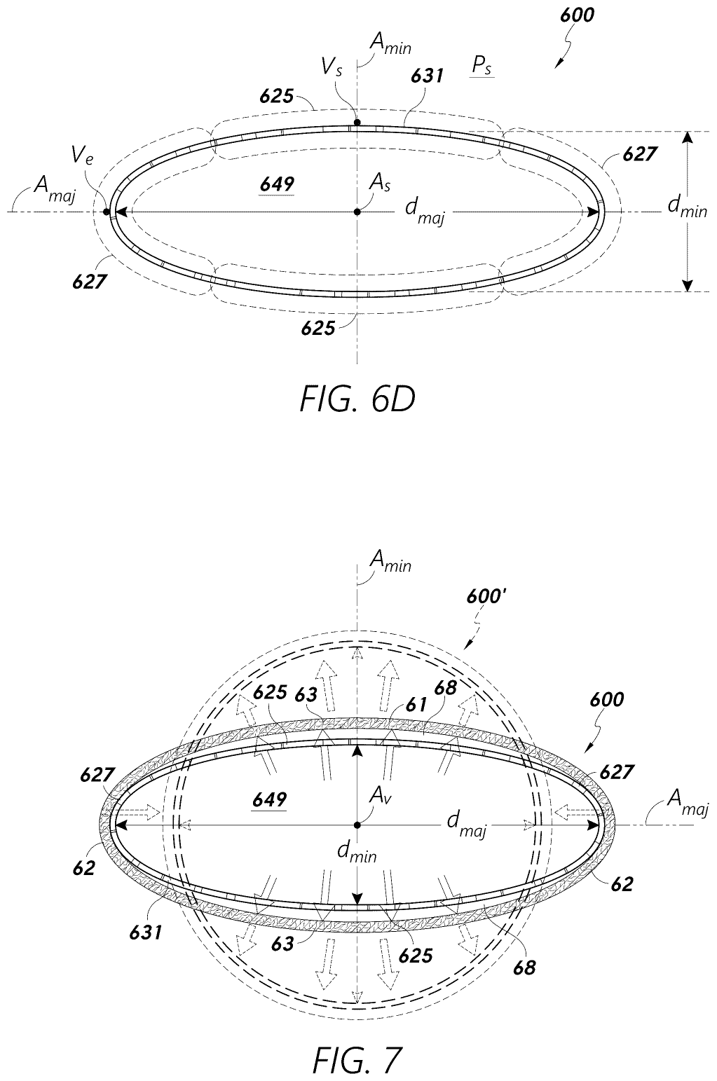

FIGS. 6A-6D show perspective, minor-axis side, major-axis side, and axial views, respectively, of a non-circular stent 600 in accordance with one or more examples. The stent may be deployable within a blood vessel lumen. However, it should be understood that example stent devices of the present disclosure may alternatively or additionally be deployable in a position around an outer surface of a target blood vessel. Although not shown for clarity in the figures of the present disclosure, it should be understood that example stents described herein may comprise one or more hooks, barbs, and/or other attachment features/means adapted to facilitate secure attachment of the stent to the tissue of the target blood vessel wall. The description of the stent 600 may be understood to relate to, and/or describe aspects of, any of the stents described herein; that is, description of aspects of any example stent of the present disclosure may be understood to be implementable in any other example stent of the present disclosure.

The stent 600, as with other stents disclosed herein, may be formed of a tubular frame 631, which may form a wall around an axial channel 649, thereby defining the channel 649. The stent 600 may be an elongate/elongated stent, in that a length L of the stent is greater than a maximum diameter dₘₐⱼ of the stent. As described herein, the frame wall 631 of the stent 600 can be considered a single, circumferentially-wrapped wall, or may be considered to comprise multiple walls, or wall segments. For example, with respect to oval stents and other non-circular stents, as illustrated in FIGS. 6A-6D, such stents may be considered to comprise sidewall segments 625 that run along relatively long sides of the stent that are aligned generally with the orientation of the major axis/dimension Aₘₐⱼ of the stent, as well as end wall segments 627, which may connect the side walls 625 on major-axis ends of the stent 600. The end walls 627 may be outwardly-curved/concave with respect to an axis Aₛ of the stent 600. In some examples, a tangent line associated with a vertex Vₑ of the end wall(s) 627 may align in parallel with the minor axis/dimension Aₘᵢₙ of the stent 600. The sidewalls 625 may be generally straight over at least a portion of a length thereof, and/or may bow/deflect inward and/or outward, cither in a resting, unpressurized state, or in conditions of hoop/wall stress on the frame 631. For example, the sidewalls 625 may bow outward such that the sidewalls 625 are concave from the perspective of the axis Aₛ of the stent 600 and convex from the perspective of the exterior of the stent 600, wherein the sidewalls 625 form a vertex Vₛ, which may be aligned with the minor axis/dimension Aₘᵢₙ of the stent 600.

Certain stent shapes are described herein, including non-circular-, oval-, peanut-, and other-shaped stents. It should be understood that such description of stent shapes refers to a shape of an axial cross-section of a stent, as depicted in the view of FIG. 6D. Although oval- and peanut-shaped stents are described, it should be understood that the principles of the present disclosure may relate to stents having any non-circular shape in at least some configurations thereof (e.g., relaxed configuration). Descriptions of stents in a relaxed configuration should be understood to relate to a configuration that a stent naturally assumes in the absence of tension on the stent wall(s) from external forces (e.g., ambient fluid pressure, physical contact forces, etc.).

The stent 600 may be considered an oval stent with respect to the shape of the axial cross-section thereof, as shown in FIG. 6D. The term “oval” is used herein according to its broad and ordinary meaning and may be used substantially interchangeably with the term “ellipse” and/or “oblong,” which terms are likewise used according to their broad and ordinary meanings. The term “oval” may be used to refer to any non-circular closed curve having major and minor axes, the major axis being greater than the minor axis. With respect to “oval”-shaped stents disclosed herein, such stents may have relatively flatter minor-axis sidewalls (compared to curved major-axis end walls), wherein the sidewalls may bow radially outward, and/or may be deflected/curved radially inward so as to produce external concavity and internal convexity in such sidewalls (e.g., forming a peanut-shaped stent). Major-axis walls of an oval stent as described herein may be considered wall portions of a stent that are intersected by a major axis of the stent that runs through an axial center of the stent. Minor-axis walls of such oval stents may be considered wall portions that are intersected by a minor axis of the stent that runs through the axial center of the stent. The description below of the various examples of stents having non-circular cross-sectional portions/sections provide further context for interpreting the terms “oval,” “peanut,” and “non-circular” in the context of oval stents and stents having oval portions/segments. Example stents of the present disclosure may be considered to have an oval shape whether or not the shape thereof is definable by an algebraic curve. Example stents of the present disclosure may be considered oval stents when the wall(s) of the stent in an axial-cross-sectional perspective form(s) a closed curve in a plane Ps that is non-circular; one or more segments/areas thereof may resemble the outline of a portion of an egg. Oval stents of the present disclosure may include either one or two axes of symmetry of an ellipse, such as the illustrated major Aₘₐⱼ and minor Aₘᵢₙ axes. The axial cross-section of some examples of oval stents of the present disclosure may resemble the union of two semicircles on opposite sides of a rectangle, providing a shape evoking the likeness of a speed skating rink or an athletics track. In some contexts, the oval stent 600 may be referred to as a “stadium”-shaped stent, or an elongated oval.

The stent frame 631 comprises stent wall(s) defining an elongated tubular structure having a first axial end 621a with a first opening 622a. The tubular structure may further comprise a second axial end 621b with a second opening 622b, wherein the lumen/channel 649 extends between the first opening 622a and the second opening 622b, traversing the length L of the stent 600. The frame 631 and/or wall(s) thereof may comprise an open-cell structure adapted to be expanded to secure the stent 600 to a blood vessel internal (or external) wall, such as through a pressure-fit deployment, one or more tissue anchors/barbs, and/or endothelialization of the frame 631 to the vessel tissue over time.

The stent 600 may be elastically deformable between a first, non-circular configuration and a second, more-circular configuration (see dashed-line representation in FIG. 7), with the stent 600 biased toward the first configuration. In some examples, the stent frame 631 may comprise a shape-memory material, such as Nitinol. Although shown as an oval-shaped stent, the stent 600 may be any non-circular shape in a resting state thereof, such as a triangle, peanut, figure-8, and/or kidney shape.

The stent 600 may be configured to be percutaneously delivered to a blood vessel in a compressed delivery configuration. Once within the blood vessel lumen at the target deployment site, the stent 600 and/or frame 631 thereof may be configured to be radially expanded into direct surface contact with the blood vessel wall (e.g., the inner wall of an aorta segment). In some examples, the stent 600 may be configured to be expanded such that the perimeter of the stent 600 approximates and/or exceeds a perimeter of the blood vessel portion where the stent 600 is implanted, at least immediately prior to deployment/expansion of the stent. In some cases, a stent configured to expand to a greater perimeter than the native blood vessel may provide improved traction and/or resistance to migration within the blood vessel. Moreover, where the stent has a perimeter approximate to and/or slightly greater than the blood vessel perimeter may increase and/or ensure positive engagement with the blood vessel and/or maximize a compliance effect. The stent wall and/or a portion of the stent wall may be configured to be endothelialized to the blood vessel wall.

In the oval configuration shown in FIGS. 6A-6D, the stent 600 may have a cross-sectional area having a major/long axis diameter dₘₐⱼ that is substantially larger than the minor/short axis diameter dₘᵢₙ. For example, the major-axis diameter/dimension dₘₐⱼ may advantageously be at least twice as long as the minor-axis diameter/dimension dₘᵢₙ, or even 3, 4, 5, 6, or 7 times greater. The stent 600 may be configured to increase compliance of a blood vessel though constant or near-constant pressure at one or more points along a perimeter/circumference of the blood vessel to cause a change in the perimeter geometry of the vessel. For example, the blood vessel may be changed and/or moved from a non-circular/less-circular shape to a circular/more-circular shape.

The stent frame wall(s) 631 may be at least partially composed of struts 638 and/or stent openings/cells 635 between the struts 638. The dimensions and/or shape of the stent 600 may vary based on the particular application and/or target implantation anatomy. For example, the stent length Z may be selected to extend over all or a portion of an identified non-compliant length of a target blood vessel. The stent major axis dₘₐⱼ and minor axis dₘᵢₙ , when averaged, may be approximately equal to the diameter of the native blood vessel. For example, for a stent configured for deployment in an aorta, the length L may be between 1-45 cm, and in the biased oval/diastolic configuration the major axis dₘₐⱼ may be between 1-4 cm (or larger/smaller depending on the particular anatomy), and the minor axis dₘᵢₙ can be between 20-50 percent of the major axis dₘₐⱼ. However, other sizes and/or shapes are also within the scope of this disclosure.

FIG. 7 shows an axial view of the uncovered, non-circular stent 600 shown in FIGS. 6A-6D deployed within a blood vessel 61 in accordance with one or more examples. The stent 600 may be biased toward the illustrated oval and/or other non-circular relaxed/diastolic configuration (shown in solid-line), and may, when subjected to mechanical forces associated with high luminal pressure, be configured to responsively transform to a more circular systolic configuration (shown in dashed-line) such that the minor axis dₘᵢₙ approaches, and may equal, the major axis dₘₐⱼ. The cells 635 of the frame 631, formed by the arrangement of the struts 638, provide openings in the frame 631 that allow blood in the blood vessel in which the stent 600 is deployed to transfer pressure through the frame 631, to thereby load the inner diameter/surface of the blood vessel with a force resulting from increases in the luminal blood pressure as the heart beats.

Luminal pressure forces against the blood vessel wall increase the hoop stress on the blood vessel, which may force the blood vessel, and with it the stent 600, to assume a more-circular shape. The resulting hoop stress, also referred to as “tangential stress” or “circumferential stress,” from luminal pressure increase exerts radially-outward force along the blood vessel's inner circumference, such stresses/forces being tensile in nature, which can tend to cause the blood vessel to increase in diameter. However, where the elasticity of the blood vessel wall is compromised, as with the blood vessel 61, the expansion of the blood vessel diameter is limited, and therefore, the pressure increase reshapes the blood vessel without substantially increasing the circumference thereof. The blood pressure force on the blood vessel wall and resulting inward deflection of the blood vessel walls (due to outward deflection of the vessel wall portions in the area of the minor axis Amin) at the major-axis-ends 627 of the stent 600 may cause inward deflection of the ends 627 of the stent 600 to form a desired geometric change to a more-circular shape of the blood vessel and stent 600.

The transition of the stent from oval to the more-circular stent shape 600′ (shown in dashed-line in FIG. 7) causes energy to be stored in the stent frame 631 (e.g., in the elasticity and/or shape memory thereof), such that energy is returned to the blood vessel walls 62, and therefore to the blood circulation within the blood vessel segment, when the frame transitions back to the oval shape as pressure decreases. The shape memory forces of the stent 600 are advantageously sufficient to overcome the pressure forces within the blood vessel 61 to return the stent 600 to the oval configuration 600 in the presence of diastolic pressure conditions, thereby reshaping the blood vessel 61 to a non-circular (e.g., generally-oval) cross-sectional shape. With respect to implantation within the aorta or other arterial blood vessel, the systolic phase of the cardiac cycle, during which pressure levels in the aorta/arteries are relatively higher, causes the expansion of the blood vessel 61 and stent 600 to the more-circular shape (shown in dashed-line in FIG. 7), whereas the diastolic phase, which is associated with relatively lower arterial blood pressure levels, causes the elongation of the stent in the major axis Aₘₐⱼ dimension to the lower-energy oval configuration, thereby forcing the blood vessel 61 to likewise assume a more oval shape due to the blood vessel having a perimeter/circumference that is sufficiently close to the perimeter/circumference length of the stent 600 (e.g., within 20% of the length of the perimeter/circumference of the stent 600).

The natural cross-sectional shape of the aorta (and other blood vessels) may generally be circular; as explained above, for a given blood vessel wall circumference/perimeter length, the circular configuration of the blood vessel may provide the maximum area/volume within the respective blood vessel segment. Therefore, any deviation from such circular/cylindrical form of the blood vessel wall may decrease the area/volume within the respective blood vessel segment. With the oval stent 600 pushing outward to the oval configuration, the wall portions 63 may be pulled/drawn at least partially towards an axial center Aᵥ of the blood vessel 61 and/or towards each other in a manner as to cause the blood vessel 61 to form a non-circular/-cylindrical shape, such as an oval shape as shown in FIG. 7. That is, compared to the circular/cylindrical shape of the blood vessel 61, the blood vessel 61 in the oval shape has a cross-sectional area in the axial segment/area where the stent 600 is implanted that is reduced.

Due to the circumference/perimeter of the blood vessel 61 being similar to the circumference/perimeter of the stent frame 631, the configuration of the stent 600 in the oval shape causes the aortic wall 61 to assume a more oval shape to match the stent 600. However, depending on the relative size of the stent 600 to the vessel 61, the blood vessel 61 may not necessarily conform exactly to the circumference and/or shape of the oval stent 600, and some minor gap(s) 68 may be present and/or form between the frame 631 and the blood vessel wall 63 as the luminal pressure increases and pushes the vessel side walls 63 away from the frame sidewall 625.

As the stent 600 comprises a bare-frame (e.g., bare-metal) stent, wherein the frame 631 is not covered internally or externally by a fluid-tight covering, the origin of the driving force required to transition the frame 631 from the oval shape to the more circular shape 600′ may necessarily be based on hoop pressure/stress against the vessel inner diameter. Coverings and other components of example implant devices of the present disclosure can comprise any type of biocompatible material, such as, but not limited to, expanded polytetrafluoroethylene (ePTFE), polyester, polyurethane, fluoropolymers (e.g., perfluoroelastomers and the like), polytetrafluoroethylene, polyethylene terephthalate (Dacron), silicones, urethanes, ultra-high molecular weight polyethylene, aramid fibers, and combinations thereof.

The luminal pressure exerts radial outward force against the vessel internal wall, wherein such forces indirectly act against the major axis dₘₐⱼ of the stent 600 to force outward deflection of the sidewalls 625 towards the circular shape 600′. For example, as the blood pressure increases in the vessel 61, the hoop stress on the blood vessel walls may force the side wall portions 63 of the blood vessel 61 to deflected radially outward towards a more uniform circular shape of the blood vessel 61, wherein the stiffness of the blood vessel wall causes the outward deflection of the sidewalls 63 to pull radially inward on the end walls 62 of the blood vessel, thereby applying radially-inward pressure on the end walls 627 of the stent frame 631. Such pressures force inward deflection of the walls 627 of the stent 600, thereby allowing the sidewalls 63 of the blood vessel 61 to deflect outwardly towards the circle dimension as pushed by the stent sidewalls 625. The walls 63 may be forced outward by outward radial deflection of the sidewalls 625 of the stent resulting from the translation/transfer of mechanical force from the end walls 627 to the side walls 625 causing a radially-outward moment/force in the side walls 625. The force vector pushing radially inwardly on the side wall 627 creates peripheral tension/stress (e.g., ring/hoop stress) in the sidewalls 625, which causes the outward deflection thereof. Therefore, with a bare-frame stent, transition force for transitioning the stent to a circular shape from an oval shape may necessarily be implemented from two primary contact points in the areas of the sidewalls 627 and/or vertices Vₑ associated with the major-access ends of the stent frame 631. Such forces inwardly compress the major axis dₘₐⱼ of the stent 600. The force vectors pushing against the ends 627 of the frame 631 may necessarily be required to be sufficient to overcome the concentrated resistance at the sides 627 and/or vertices Vₑ of the frame 631. In view of the elongated shape of the oval frame 631, the radially-outward resistive force of the frame 631 may be greater at the major-axis ends 627 than at the minor axis walls 625, such that a relatively substantial force may be required to cause the inward deflection of the frame ends 627.

As the pressure in the blood vessel 61 increases (e.g., in connection with the systolic phase of the cardiac cycle), the plastically-deformable nature of the stent 600 allows for the sidewalls 625 of the frame 631 to be pushed outward to accommodate the shortening of the stent 600 in the major axis dimension Aₘₐⱼ. When the ends 627 of the stent 600 are brought closer together (as pushed radially inward by the blood vessel walls 62), the stent 600 allows/compels the blood vessel 61 to assume a more circular cross-sectional shape. That is, the stent 600 improves cardiac perfusion by causing a decrease in cross-sectional blood vessel area during diastole relative to systole due to the reshaping of the blood vessel 61 caused by the lengthening of the major diameter dₘₐⱼ of the stent 600. For example, for an insufficiently-compliant blood vessel, the outer wall/perimeter of the blood vessel may generally not change sufficiently between systole in diastole due to a lack of stretching of the blood vessel wall. Therefore, with a typical circular cross-sectional shape throughout the cardiac cycle, the cross-sectional area of the blood vessel may not change sufficiently between cardiac phases, resulting in poor perfusion. Examples of the present disclosure solve this problem by leveraging the principle that a non-circular cross-sectional shape will have lesser area than a circular cross-sectional shape having the same perimeter length. In particular, implant devices presented herein are configured to achieve improved perfusion by changing the cross-sectional area of the aorta (or other blood vessel) from a more-circular to a less-circular (e.g., oval) shape as the cardiac cycle transitions from systole to diastole. The compliance-enhancing stent 600 is expanded in the minor-axis dimension dₘᵢₙ in response to higher-pressure conditions to allow the vessel 61 to assume a more circular shape, after which the shape memory bias in the stent 600 pushes the vessel walls 62 back outward as pressure drops, thereby pushing blood through the vessel and improving perfusion.

The stent frame 631 may be inclined to experience tissue in-growth in one or more areas thereof. For example, the end walls 627 may be relatively stationary relative to the blood vessel walls 62 throughout the cardiac cycle due to the relatively constant contact between the stent side walls 627 and the blood vessel sidewalls 62 between oval and circular shapes of the stent 600. Conversely, the sidewalls 625 of the frame 631 and the sidewalls 63 of the blood vessel may tend to come into contact with one another and separate in a cyclical manner as the blood pressure forces the blood vessel sidewalls 63 outward and away from the stent sidewalls 625 during portion(s) of the high-pressure phase/stage of the cardiac cycle. Therefore, tissue ingrowth may occur primarily between the blood vessel sidewalls 62 and the stent sidewalls 627 in situations in which tissue ingrowth occurs. That is, due to the hoop stresses on the blood vessel 61 causing cyclic detachment preventing tissue ingrowth with respect to the longer and/or straighter sidewalls of the stent and blood vessel, tissue ingrowth can grow more in the major-axis regions where contact between stent and blood vessel is relatively more continuous/constant throughout cardiac cycling.

As with any of the examples disclosed herein, the stent 600 can be configured to deflect from the oval shape to a more-circular shape in the presence of threshold blood pressure levels greater than 80 mmHg, such as blood pressure levels greater than 90 mmHg (e.g., between 90-120 mmHg).

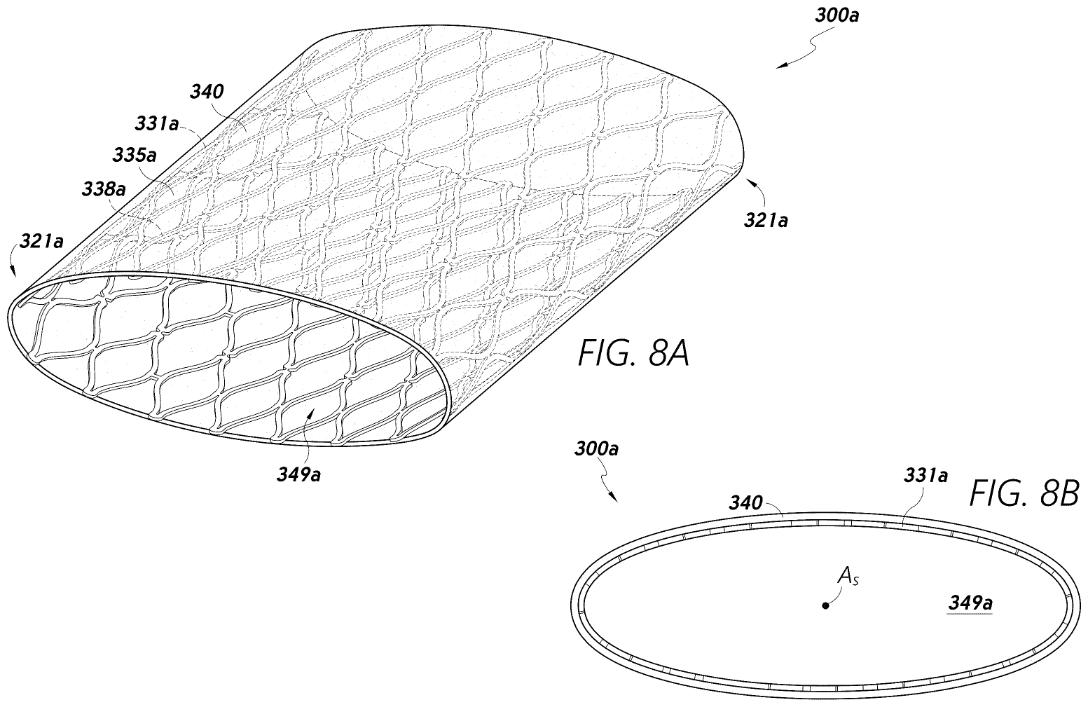

FIGS. 8A and 8B show perspective and axial views, respectively, of a non-circular stent 300a having an outer covering 340 in accordance with one or more examples. The stent 300a may have the features of any of the example non-circular stents disclosed herein. For example, the stent 300a may comprise a stent frame 331a formed of an arrangement of struts 338a arranged to form a plurality of rows and/or columns of cells 335a. Providing an outer cover/covering 340 can be advantageous as providing a mechanism to limit contact between the struts 338a of the frame 331a and the surrounding blood vessel wall, thereby reducing risks of irrigating the blood vessel tissue and/or facilitating thrombosis.

An outer surface of at least a portion of the frame 331a and/or struts 338a thereof is/are covered by a covering 340, which is advantageously fluid-tight, such that pressurized blood against the surface of the covering 340 is blocked or impeded from passing through the covering. The covering 340 may be designed to cause luminal pressure within a channel 349a of the stent 300a to exert distributed force against the covering 340, which is advantageously fixed to the frame 331a and one or more portions/areas thereof, thereby translating such luminal forces into hoop stress/tension in the covering 340 and/or frame 331. The covering 340 prevents blood in the channel 349 from propagating/passing through the cells 335a to contact and/or exert force on the blood vessel walls outside of the stent frame 331.

The outer covering 340 may have sufficient thickness and/or other characteristics to protect the blood vessel wall from damage caused by the frame 331a, such as imprinting of the frame 331a on the vessel wall, which may cause tissue necrosis or other damage to the vessel. Furthermore, the covering 340 can promote tissue ingrowth and/or other attachment or secure frictional interference with the blood vessel wall to secure the stent 300a in-place in the target position in the blood vessel.

As with any of the stent coverings disclosed herein, the covering 340 can span the entire circumference of the stent frame 331, or only a portion of the circumference. For example, in some implementations, the covering 340 is composed of a plurality of sub-sections of covering that cover respective regions around the perimeter of the stent. Such covering segments may be circumferentially spaced/offset from one another. In some implementations, covering segments may be positioned in areas of a major axis of an oval stent to promote ingrowth of the stent with the surrounding blood vessel tissue at major-axis ends of the stent. Alternatively or additionally, covering segments may be positioned on minor axis sidewalls to facilitate the outward deflection of such walls in the presence of sufficiently high luminal pressure within the flow channel of the stent.

The covering 340, as with any other covering disclosed herein, may comprise any suitable or desirable material. For example, the covering 340 may comprise a sheet of polymer, such as PTFE, ePTFE, TPU, or other type of polymer. In some implementations, stent coverings of examples of the present disclosure comprise fabric configured to induce and/or encourage tissue ingrowth with the covering layer(s), or alternatively designed to impede tissue-ingrowth with respect to an area or region of the stent for which endothelialization is not desirable.

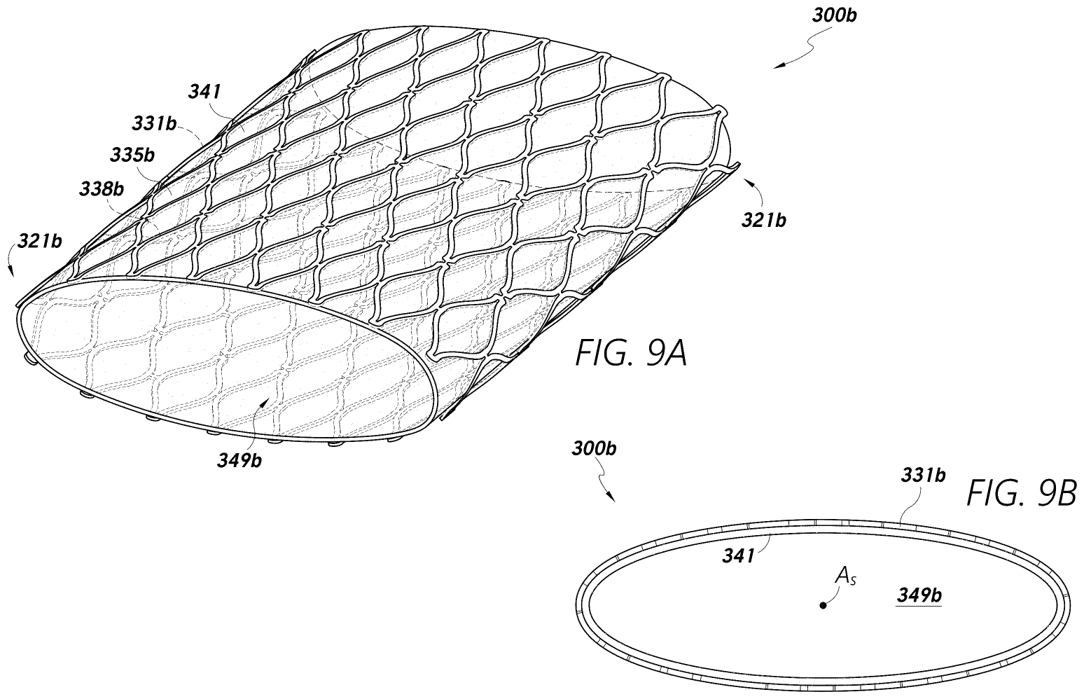

FIGS. 9A and 9B show perspective and axial views, respectively, of a non-circular stent 300b having an inner covering 341 in accordance with one or more examples. The stent 300b may be similar to the stent 300a of FIGS. 8A and 8B, with the exception that the covering 341 is disposed at least partially within the stent frame 331, such that the covering is disposed on and/or lining a radially-inner surface of the struts 338b of the frame 331b.

In some implementations, the covering 341 may adhere otherwise be secured to one or more portions of the inner surface/diameter of the frame 331, wherein portions of the covering 341 that span between struts 338b and across cells 335b of the frame 331b fill the spaces between the struts 338b in the cells 335b. That is, portions of the covering 341 may deflect radially outward in the cell spaces 335b, such that such portions of the covering 341 are disposed/sunk/nested in the opening/recesses provided by the cells 335b. Disposing the covering 341 on the inner layer may be preferable to outer covering implementations as the pressure forces within the channel 349b will push outward against the covering, which in-turn presses outwardly against the inner diameter of the stent frame 331b. For example, if only an outer covering is implemented, radially-outward force against the covering may be prone to push the covering away from the frame where the covering is not sufficiently secured to the frame. Inner coverings, conversely, may not need to be as securely fixed to the frame to avoid separation of the frame and covering due to the pressing of the covering against the frame as opposed to pushing the covering away from the frame.

The outer covering 340 of the stent 300a and/or the inner covering 341 of the stent 300b may be configured to promote hemostasis scaling between the stents and the blood vessel in which they are implanted. Such sealing may advantageously occur at least at end region/areas 321 of the stent 300. Stent coverings described herein may comprise textiles or other materials configured to promote endothelization, which may help to secure the stent 300 to the blood vessel, as well as provide sealing functionality to prevent blood from passing on an outer diameter of the stent 300. Where blood is permitted to pass on an outside of the stent 300, such blood may provide pressurized fluid that reduces the pressure gradient between the flow channel of the stent and the exterior thereof, thereby inhibiting reshaping/expansion of the stent in accordance with aspects of the present disclosure.

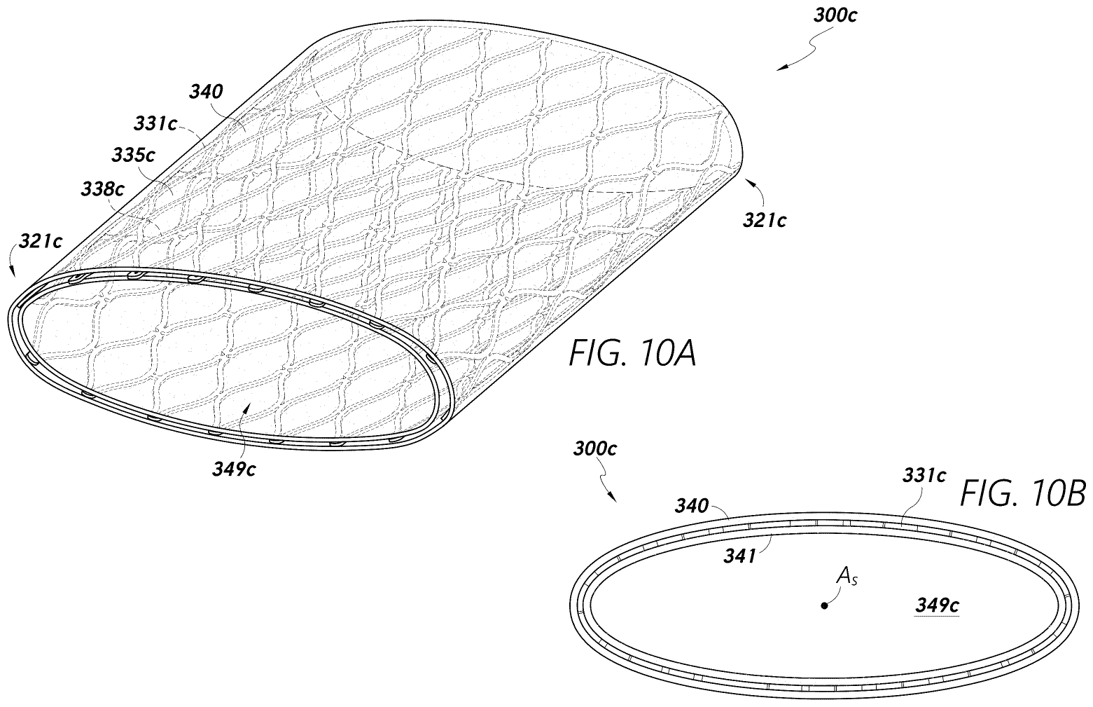

FIGS. 10A and 10B show perspective and axial views, respectively, of a non-circular stent 300c having outer 340 and inner 341 coverings in accordance with one or more examples. The outer 340 and inner 341 coverings may sandwich the stent frame 331c therebetween. The implementation of two layers of covering on inner and outer sides/portions, respectively, of the stent frame 331c may provide increased sealing functionality and/or fluid-tightness of the stent 300c relative to single-covering/layer implementations. In some examples, the inner 341 and outer 340 covering layers may adhere to one another in the spaces/areas between the struts 338c, such as within the cells 335c, which may advantageously improve the adhesion/attachment of the covering to the stent frame 331c. That is, the adhesion between the layers of covering may provide the attachment mechanism/means for attaching to the stent frame 331c. In some implementations, the covering layers 341, 340 are formed of a single unitary sheet/layer of covering material that wraps over one or more ends 321c of the frame 331c and doubles-back on itself to provide the two-layer sealing configuration/structure shown.

With inner 341 and outer 340 layers of covering, the inner layer 341 may fill the spaces of the opening/cells 335c, providing advantageous sealing characteristics thereby, whereas the outer cover layer 340 may provide a protective layer between the stent frame 331c and the outer blood vessel wall. The inner 341 and outer 340 layers of covering may comprise a common material and/or different materials. For example, the inner layer 341 may comprise material designed to provide advantageous sealing characteristics, whereas the outer layer 340 may provide a material/surface that promotes tissue ingrowth and/or protection for the device 300c. In some implementations, the outer cover layer 340 may be thicker than the inner layer 341, or vice versa. The outer layer may protect the vessel wall from trauma or imprinting of the stent frame 331c, which may prevent tissue necrosis or other damage to the blood vessel tissue.

Although described as stents, it should be understood that any of the devices 300 of FIGS. 8-10 may be implemented and/or implanted as grafts that replace resected blood vessel segment(s). For example, end portions of the devices 300 may be sutured or otherwise attached to open ends of blood vessel segments such that at least a portion of the length of the device 300 spans a distance between the blood vessel segments.

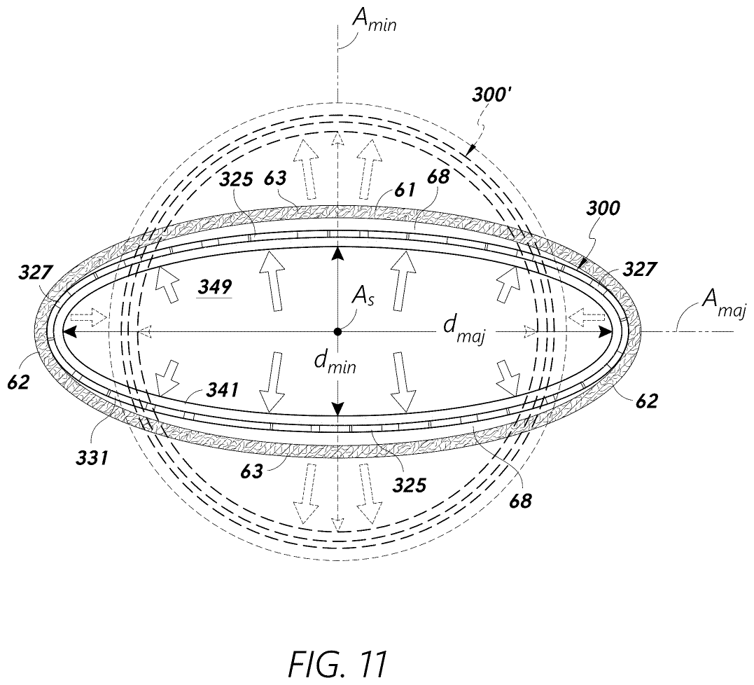

FIG. 11 shows a covered non-circular stent 300 deployed within a blood vessel 61 in accordance with one or more examples. The stent 300 may have any of the features of the frames 300a, 300b, 300c described above, and the description of FIG. 11 can be understood with reference to the description of such devices in connection with FIGS. 8A, 8B, 9A, 9B, 10A, and 10B. The stent 300 may be biased toward the illustrated oval and/or other non-circular relaxed/diastolic configuration (shown in solid-line), and may, when subjected to radially expansive forces, be configured to responsively transform to a more-circular systolic configuration (shown in dashed-line) such that the minor axis dim of the stent 300 approaches, and may equal, the major axis dₘₐⱼ. The cells 335 of the frame 331 of the stent 300 may be covered within and/or without by fluid-tight covering 341 (shown as an inner covering), such that the openings of the cells 335 are closed to pass-through fluid and prevent blood in the blood vessel 61 in which the stent 300 is deployed and within the channel 349 of the stent 300 from transferring pressure through the frame 331. Therefore. intraluminal pressure within the flow channel 349 of the stent 300 loads against the frame 331 (rather than directly against the blood vessel walls 63) to provoke reshaping thereof. Although the covering 341 is shown as an inner covering on an inside of the frame 331, it should be understood that the covering 341 may be disposed outside of the frame 331 or both within and without the frame 331.

The blood-pressure-induced force against the covering 341 and/or stent frame 331 increases the hoop stress on the frame 331 and/or covering 341, which may force the frame 331, and with it the blood vessel 61, to assume a more-circular shape. The resulting hoop stress, also referred to as “tangential stress” or circumferential stress,” from increase in luminal pressure exerts radially-outward force along the cover's and/or frame's circumference, such stresses/forces being tensile in nature, which can tend to cause the frame 331 to increase in diameter along the minor axis Amin. The blood pressure force on the cover/frame of the stent 300 and resulting deflection of the of the frame walls 325 in the minor-axis dimension Aₘᵢₙ may cause inward deflection of the ends 327 of the stent 300 to form a desired geometric change to a more-circular shape of the stent 300 and blood vessel 61.

The transition of the stent from oval 300 to the more-circular 300′ stent shape (shown in dashed-line in FIG. 11) causes energy to be stored in the stent frame 331 (e.g., in the elasticity and/or shape memory thereof), such that energy is returned to the blood circulation when the frame 331 transitions back to the oval shape 300 as pressure decreases due to the decrease in stent channel 349 volume when moving from circular to oval cross-sectional shape. The shape memory forces of the stent 300 are advantageously sufficient to overcome the pressure forces within the channel 349 to return the stent 300 to the oval configuration in the presence of diastolic pressure conditions, thereby reshaping the blood vessel 61 to a non-circular (e.g., generally-oval) cross-sectional shape. With respect to implantation within the aorta or other arterial (or venous) blood vessel, the systolic phase of the cardiac cycle, during which pressure levels in the aorta/arteries are relatively higher, causes the expansion of the blood vessel 61 and stent 300 to the more-circular shape (shown in dashed-line in FIG. 11), whereas the diastolic phase, which is associated with relatively lower arterial blood pressure levels, causes the elongation of the stent in the major axis Aₘₐⱼ dimension to the lower-energy oval configuration, thereby forcing the blood vessel 61 to likewise assume a more-oval shape due to the blood vessel having a perimeter/circumference that is sufficiently close to the perimeter/circumference length of the stent 300 (e.g., within 20% of the length of the perimeter/circumference of the stent 300).

The natural cross-sectional shape of the aorta (and other blood vessels) may generally be circular; as explained above, for a given blood vessel wall circumference/perimeter length, the circular configuration of the blood vessel may provide the maximum area/volume within the respective blood vessel segment. Therefore, any deviation from such circular/cylindrical form of the blood vessel wall may decrease the area/volume within the respective blood vessel segment. With the oval stent 300 pushing outward to the oval configuration, the wall portions 325 may be pulled/drawn at least partially towards an axial center Aₛ of the stent 300 and/or towards each other in a manner as to cause the stent frame 331 to form a non-circular/-cylindrical shape, such as an the oval/ellipse/slot shape shown in FIG. 11. That is, compared to the circular/cylindrical shape 300′ of the stent, the stent 300 in the oval shape has a cross-sectional area that is reduced.

Due to the circumference of the blood vessel 61 being similar to the circumference of the stent frame 331, the configuration of the stent 331 in the oval shape causes the or aortic walls 61 to assume a more oval shape to match the stent 300. However, depending on the relative size of the stent 300 to the vessel 61, the blood vessel 61 may not necessarily conform exactly to the circumference and/or shape of the oval stent 300, and some minor gap(s) 68 may or may not be present between the stent walls 325 and the vessel walls 63. The covering 341 and/or frame 331 may facilitate a hemostatic seal between the stent 300 and the vessel at least with respect to axial end portions of the stent 300.

Unlike with bare-frame stents that do not include fluid-tight coverings, the endoluminal pressure forces in the channel 349 of the covered stent 300 are distributed over/against the covering 341 and/or frame 331 to directly reshape the stent 300, rather than indirectly reshaping the stent 300 through force on a finite number of end contact/pressure points of the frame 331. As the pressure in the channel 349 increases (e.g., in connection with the systolic phase of the cardiac cycle), the plastically-deformable nature of the stent frame 331 allows for the sidewalls 325 of the frame 331 to be pushed outward along with the shortening of the stent 300 in the major axis dimension Aₘₐⱼ. When the sidewalls 325 are deflected outward, the channel 349 assumes a more circular cross-sectional shape. That is, the stent 300 improves cardiac perfusion by causing a decrease in cross-sectional blood flow channel area during diastole relative to systole due to the reshaping of the channel 349 caused by the lengthening of the major diameter dₘₐⱼ of the stent 300 and the shortening of the minor diameter dₘₐⱼ. For example, for an insufficiently-compliant blood vessel, the outer wall/perimeter of the blood vessel may generally not change sufficiently between systole in diastole due to a lack of stretching of the blood vessel wall. Therefore, with a typical circular cross-sectional blood flow channel shape throughout the cardiac cycle, the cross-sectional area of the flow channel may not change sufficiently between cardiac phases, resulting in poor perfusion. Examples of the present disclosure solve this problem by leveraging the principle that a non-circular cross-sectional shape will have lesser area than a circular cross-sectional shape having the same perimeter length. In particular, implant devices presented herein are configured to achieve improved perfusion by changing the cross-sectional area of the blood flow channel from a more-circular to a less-circular (e.g., oval) shape as the cardiac cycle transitions from systole to diastole. The compliance-enhancing stent 300 is expanded in the minor axis dimension dₘᵢₙ in response to higher-pressure conditions to allow the blood flow channel to assume a more circular shape, after which the shape memory bias in the stent 300 pushes the stent frame walls back outward as pressure drops, thereby pushing blood through the stent and improving perfusion.

The covering 341 and/or frame 331 may be inclined to experience tissue ingrowth in one or more areas thereof. For example, the portion of the covering 341 and/or frame 331 in the area of the end walls 327 may be relatively stationary relative to the blood vessel walls 62 throughout the cardiac cycle due to the relatively constant contact between the covering 341 and/or frame 331 and the blood vessel sidewalls 62. Conversely, the covering 341 and/or frame 331 in the area of the sidewalls 325 of the frame 331 and the sidewalls 63 of the blood vessel may tend to come into contact with one another and separate in a cyclical manner as the shape memory of the frame 331 forces the stent sidewalls 325 inward and away from the blood vessel sidewalls 63 during portion(s) of the low-pressure phase/stage of the cardiac cycle. Therefore, tissue ingrowth may occur primarily between the blood vessel sidewalls 62 and the stent frame 331 and/or covering 341 in the areas of the frame sidewalls 327 in situations in which tissue ingrowth occurs.

Covered oval/non-circular stents of the present disclosure can advantageously spread elevated luminal pressure forces in a more homogenous manner across/along a circumference of the covering and/or frame of the stent, thereby forcing the covered stent itself, and thereby the blood vessel wall pressed against the stent, to assume a more circular shape. Where covering is applied properly to the stent frame, proper hemostasis may be provided at one or both axial ends of the stent. In such cases, the luminal pressure forces do not exert hoop stress directly on the blood vessel around the stent, but rather act against the covering itself, such that the pressure can be utilized to directly push/pull the minor-axis sidewalls of the stent. Although the majority of the pressure forces are exerted on the covering and/or frame of the stent, as opposed to the blood vessel wall in the axial area of the stent, the blood vessel may nevertheless be reshaped by the stent due to the conformity of the blood vessel with the stent shape.

During low-pressure (e.g., diastole), the stent 300 may physically interact with the blood vessel 61 in an at least partially isolated manner through interaction with the blood vessel walls 62 in the areas of the major-axis ends 327 of the stent, which push outward from the axis Aₛ to force the vessel 61 into an oval shape. The diameter of the stent 300 may be similar to and/or slightly greater than the corresponding diameter of the blood vessel 61, which may serve to facilitate secure positioning of the stent 300 within the blood vessel 61.

With respect to any of the example stents disclosed herein, although some such examples are described as having frames composed of various arrangements of struts that form cells, it should be understood that such stent devices may alternatively (or additionally) be formed of sheets of material, rather than frame struts forming open cells. In such examples, the functionality of the covering of the stent frame that is described in detail herein, which serves to present a relatively broad surface against which luminal pressure forces can press/push to reshape an oval/non-circular stent in accordance with aspects of the present disclosure, may be achieved by the planar surface of the non-open frame.

Although the blood vessel 61 is shown, it should be understood that the covered stent 300 can be implanted as a graft, wherein at least a portion of the length of the device is not covered by blood vessel, but rather is open in the chest cavity or other anatomical chamber/area.

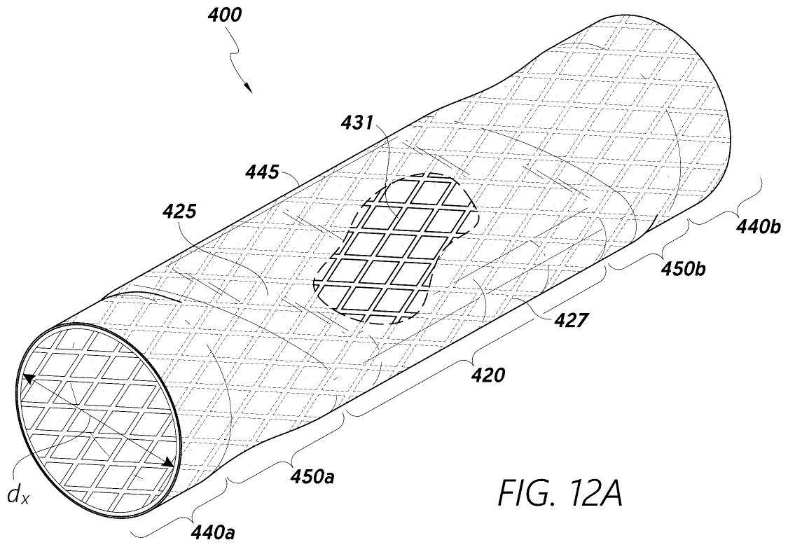

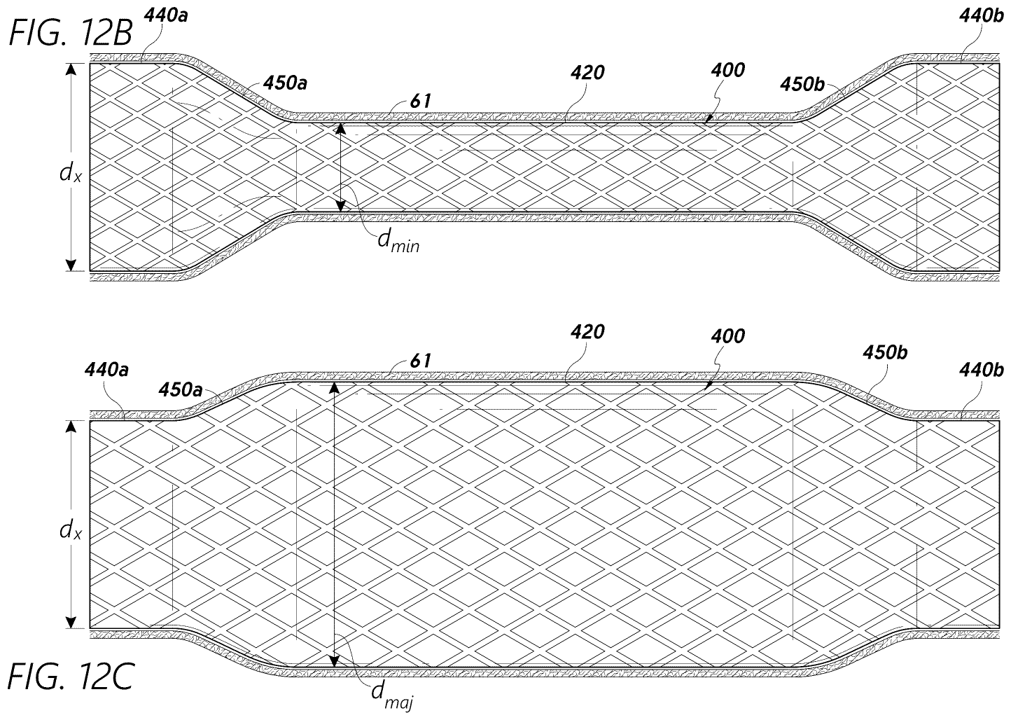

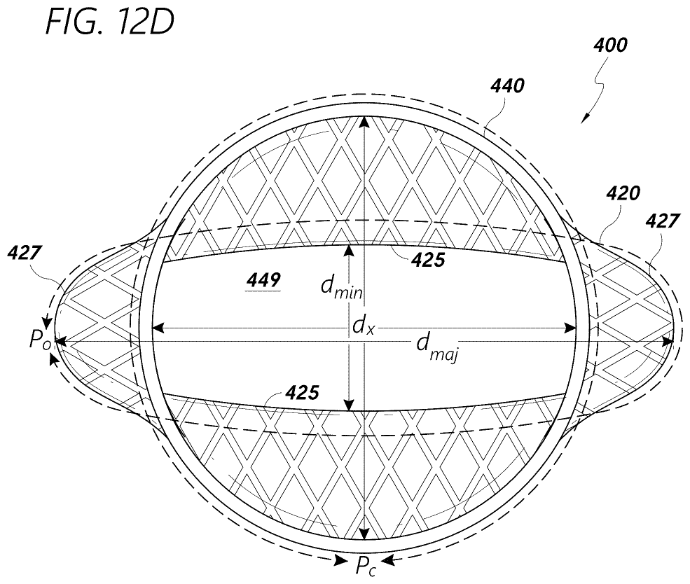

FIGS. 12A-12D show perspective, side, and axial views, respectively, of a stent 400 having circular end portions 440 and a non-circular (e.g., oval) medial portion 420 in accordance with one or more examples. Although the device 400 is described below as a stent, it should be understood that, as with any other device disclosed herein, the device 400 may be implemented and/or implanted as a graft that replaces a resected blood vessel segment or other gap between blood vessel segments. For example, the end portions 440 of the devices 400 may be sutured or otherwise attached to open ends or openings of/in blood vessel segment(s), such that at least a portion of the length of the medial portion 420 of the device 400 spans a distance between the blood vessel segments and/or is not entirely endovascular when implanted.

As described in detail throughout the present disclosure, oval-shaped and other non-circular stents can be utilized to improve compliance characteristics of a target blood vessel or blood vessel segment. Due to various hemodynamic considerations, as referenced above, it may be desirable to implement such stents in a manner such that a hemostasis seal is present/facilitated on axial ends of the stent. Furthermore, due to the natural circular/cylindrical shape of the blood vessel, oval stents of some examples may not fit as securely within a target blood vessel as compared to traditional circular/cylindrical stents of the same size. For example, while a circular/cylindrical stent may generally distribute contact with the blood vessel wall evenly around the circumference thereof, such that contact between the stent and the blood vessel is not concentrated in specific contact points around the circumference of the stent, oval stents may have a tendency to contact the blood vessel primarily at the major axis ends of the stents oval form, as described in detail above. Furthermore, due to the dynamic reshaping of certain oval stents as described herein, minor axis sidewalls of the oval stent may become cyclically spaced/separated from the adjacent blood vessel wall, further compromising the secure axial fixation of the stent within the blood vessel and/or the hemostatic seal/barrier between the stent and the vessel wall. Therefore, oval stents in accordance with aspects of the present disclosure can benefit from utilization with certain vessel-anchoring features to further facilitate/improve the axial fixation of such stent devices in the target blood vessel segment and/or promote hemostasis between the stent and the blood vessel wall and/or prevent the collection of blood between the stent and the blood vessel wall.

In some implementations, stent device of the present disclosure include stent/frame portions that have a circular/cylindrical relaxed shape/form, wherein such stent portions can be integrated with oval portion(s) of the stent in some manner, such that the circular portion(s) of the stent serve to securely hold the stent in place in the blood vessel, whereas the oval portion(s) of the stent can function to increase blood flow (e.g., diastolic flow) through the stent as the oval stent portion transitions between oval and circular/cylindrical configurations in response to changing pressure conditions, as described in detail herein.

The stent 400 includes circular stent portions 440a, 440b, which are associated with respective axial ends of the stent 400. Although circular portions 440 are shown on both axial ends of the stent 400, it should be understood that circular-oval stent devices of the present disclosure may include only a single circular portion on one end of the device in some cases. The circular stent portions 440 can have any suitable or desirable axial length. In some implementations, the circular portions 440 have a length (in the dimension L) that is less than the oval portion 420. For example, the oval portion 420 may have a length that is at least twice as long as either of the circular portions 440, or twice as long as the combined lengths of the circular portions 440.

The circular portions 440 may be formed of portions of a stent frame 431, which may be integrally formed with the frame portion forming the oval portion 420 of the stent 400. For example, the frame 431 may transition between the circular shape of the circular portions 440 and the oval shape of the oval portion 420. The frame 431 may transition in a relatively smooth/gradual manner from the shape of the oval segment 420 to the circular shape of the circular segments 440 in transition portions/segments 450 of the stent 400. That is, with respect to the flat/long sidewalls 425 that run in the major axis dimension of the oval portion 420, the diameter of the frame 431 may transition from a narrow minor-axis diameter dₘᵢₙ to the circular diameter dₓ of the circular portions 440 moving axially from the oval portion 420 towards the circular end portions 440. Furthermore, with respect to the curved/short end walls 427 of the oval portion 420 that curve around the major axis ends of the oval form thereof, the diameter dₘₐⱼ associated therewith may transition from the relatively long dimension dₘₐⱼ to the relatively shorter circular diameter dₓ moving from the oval portion 420 through the transition portions 450 to the circular portions 440. In some examples, as shown in FIGS. 12A-12D, the diameter dₓ of the circular portions 440 may be less than the major diameter dₘₐⱼ of the oval portion 420, but greater than the minor diameter dₘᵢₙ of the oval portion 420.

As shown in FIG. 12A, the stent 400 may include a covering 445, which is illustrated on an outer surface of the stent 400, although it should be understood that such covering 445 may be disposed within the frame 431 on an inner side/diameter thereof and/or within and without the frame 431, as with other examples of the present disclosure. For clarity, the covering 445 is shown only in FIG. 12A. However, it should be understood that the covering 445 may be present in any example stents disclosed herein. Furthermore, while a stent frame 431 and covering 445 are shown in FIG. 12A, wherein the covering provides a fluid-tight surface against which pressurized blood within the channel 449 of the stent 400 can press to reshape the oval portion 420 of the stent 400, it should be understood that in some examples, the stent 400 comprises a planar/sheet form that is fluid tight in one or more portions thereof, wherein such planar/sheet form provides the structure/frame of the stent 400 and substitutes for the illustrated strut-based frame 431 and covering 445.

The stent 400, which may be a covered or bare-frame stent, is formed with the central/medial oval-shaped segment 420, which assumes and oval or other non-circular shape at least in a free/relaxed state thereof, with both ends of the stent having circularly-shaped forms so as to fully-engage with the surrounding vascular wall. In some implementations, the end portions 440 can be relatively oversized to conform with and press against the blood vessel walls, which can improve hemostasis, thereby restricting blood flow to solely through the stent's lumen 449. The oversizing of the circular portions 440 relative to the native blood vessel can further help the stent 400 resist migration in either axial direction.

The stent 400 and/or frame 431 thereof may have the same perimeter length P_{c} in the circular portions 440 as in the oval portion 420 (perimeter Pₒ shown in FIG. 12D). The transition segment/portions 450 may likewise have the same perimeter as the end 440 and oval 420 segments. The perimeter P_{c} of the circular portions 440 may be selected to match the circumference of the target blood vessel segment. For example, the perimeter P_{c} may be within 10% of the length of the circumference of the target blood vessel in the implantation area. The circumference/perimeter P_{c} of the stent may be slightly larger than that of the target blood vessel prior to deployment of the stent 400, to thereby promote secure attachment of the stent 400 to the blood vessel.

Where the circumference P_{c} of the circular segments 440 and the circumference P_{c} f the oval segment 420 are substantially the same and matched to that of the blood vessel, the blood vessel 61 may be forced to assume the same shape as the stent 400 in the respective areas thereof, such that the blood vessel walls 61 are pressed and/or disposed relatively tightly around the stent's outer walls. For example, as shown in FIGS. 12B and 12C, the blood vessel 61 may assume the circular shape around the circular portions 440 in the areas adjacent thereto and transition to the oval shape of the oval segment 420 in such area. With the blood vessel 61 being forced around the shape of the stent 400 in a relatively tight configuration, any blood disposed on the outside of the stent between the stent 400 and the blood vessel wall 61 during deployment of the stent 400 may be forced out from around the stent 400, such that little or no blood is present between the stent 400 and the blood vessel 61 after deployment/implantation of the stent 400.

When the stent 400 is implemented with a covering 445, the circular end-portions 440 can serve as funnels that restrict blood flow to flowing through the channel of the medial portion 420, and not flowing around the outside of the medial portion 420. However, as the circular end portions 440 may not be configured to change in shape throughout the cardiac cycle as pressure changes, such portions may not contribute to the stent's compliance-enhancing, vessel-reshaping effect. Moreover, the circular shape of the end portions 440 may cause the end portions to serve as stationary harnessing portions that may limit the movement of the central/medial 420 portion between systolic and diastolic phases. Furthermore, the relatively abrupt transition from the enlarged circular shape of the end portions 440 to the narrower profile of the non-circular medial portion 420 shaped portion may result in certain local flow disturbances, and/or pose manufacturing complications associated with increased deformations/strains. Therefore, it may be desirable to implement the end portions 440 as non-circular portions in some implementations.

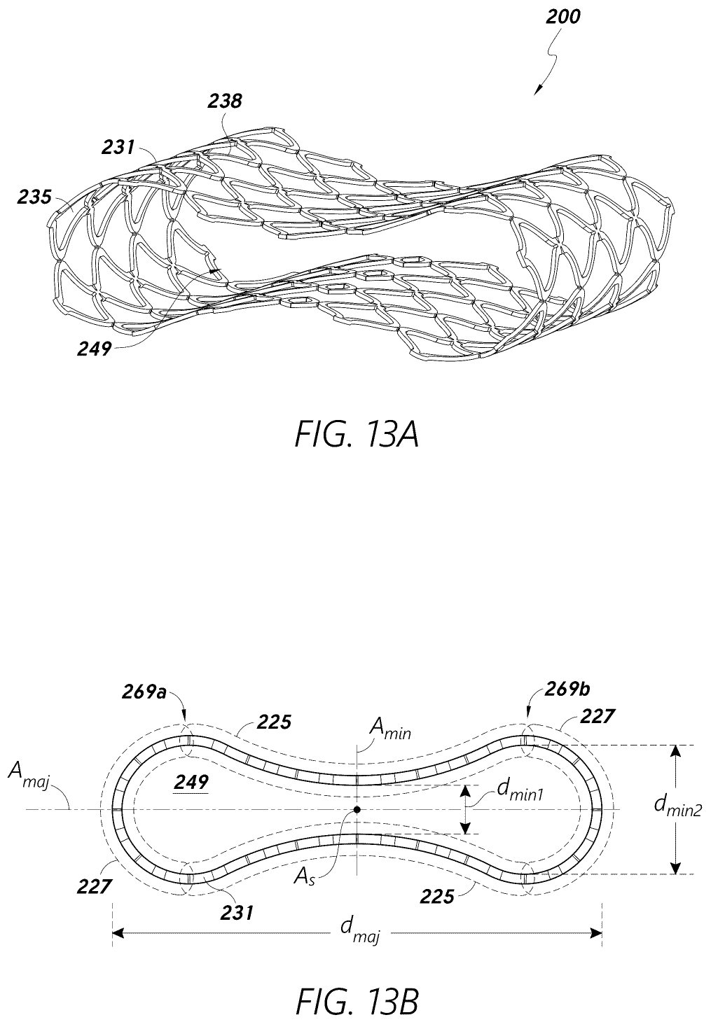

FIGS. 13A and 13B show perspective and axial views, respectively, of a non-circular stent 200 having inwardly-deflected sidewalls 225 in accordance with one or more examples. The stent 200 may represent an example implementation of any of the non-circular/oval stents disclosed herein, or portion(s) thereof. The stent 200 forms/defines an elongate/elongated tubular member, as shown in FIG. 13A, which forms a blood flow lumen 239.

The shape of the stent 200 deviates from the stent shown in FIGS. 6A-6D only in that the minor-axis (e.g., relatively flat and/or long) sidewalls 225 deflect to a greater degree towards the center/axis As of the stent 200 in a relaxed state. The resulting shape may resemble that of an hourglass and/or peanut shape with respect to the axial cross-section shown in FIG. 13B. The stent 200 may have a major-axis diameter/dimension dmaj that is greater than a minor axis diameter/dimension dmin of the oval cross-section. As with other oval stents disclosed herein, the stent 200 is configured to change in perimeter geometry to transition from the illustrated non-circular shape to a more-circular configuration in response to the application of certain mechanical forces on one or more points/portions thereof.

In some examples, the stent 200 may be biased to a shape having a minor axis dimension dₘᵢₙ that is non-constant along the major axis Aₘₐⱼ dimension, which forms an externally-concave/internally-convex surface/form with respect to the minor axis sidewalls 225. For example, the minor-axis sidewalls 225 may have a diameter dimension dₘᵢₙ₁ at a center thereof (with respect to the major axis dimension Aₘₐⱼ) that is less than the diameter/dimension dₘᵢₙ₂ at/towards the end portions of the minor-axis sidewalls 225. Non-circular stents of the present disclosure that have externally-concave/internally-convex minor-axis sidewalls as shown in FIGS. 13A and 13B are referred to herein as peanut-shaped stents; such peanut shape can be considered a variation of an oval, or oval-shaped, stent as described herein. The inwardly-bent walls 225 can allow for a transition reshaping between peanut-shaped, to outwardly-bowed oval shape, and ultimately to circular/more-circular shape.

The stent 200, though configured to transition to a more-circular configuration when certain forces are applied thereto, may be biased toward the illustrated non-circular, peanut diastolic shape. The stent 200 may be biased (such as being memory-set via a memory material such as Nitinol) toward the peanut shape. With the sidewalls 225 deflected inwardly, as shown, the stent frame 200 may form bulging/bulbous portions 269a, 269b on either side of the minor axis Aₘᵢₙ , wherein such portions have a diameter/dimension dₘᵢₙ₂ that is greater than the shortened diameter dₘᵢₙ₁. The resulting shape of a blood vessel in which the stent 200 is deployed when the stent 200 is in the relaxed peanut configuration can reduce a diameter/dimension of the blood vessel parallel with the minor axis Aₘᵢₙ in the area of the minor axis. The dimension/diameter of the blood vessel in the major axis dimension Aₘₐⱼ may become elongated when the wall portions 225 are brought closer together to form the peanut shape.

When deployed in a blood vessel, oval stents that are configured to transition between oval and more-circular shape/configurations may experience some degree of transition towards a more-circular shape in response to even minimum pressure conditions within the blood vessel, such as may be associated with the diastolic phase of the cardiac cycle with respect to arterial stent deployments. Therefore, between the low- and high-pressure phases of the cardiac cycle, the change in cross-sectional area of the stent may be reduced to some degree by the premature transition to a more-circular shape due to the minimum luminal blood pressure in the target vessel. The inward deflection of the stent walls 225 to produce concavity thereof in the peanut shape in the relaxed configuration of the stent 200 can bias the shape of the stent in a manner such that slight outward deflection of the walls 225 caused by minimum pressure conditions in the target blood vessel serves to flatten-out the walls 225 without deflecting-/bowing-out the walls 225 to a substantial degree rather than producing a more-circular configuration thereof. Therefore, the desired change in area/volume of the stent between low- and high-pressure phases is preserved/maintained. Therefore, the peanut configuration of the sidewalls 225 can compensate for premature rounding-out/transformation of the stent into more-circular shapes.

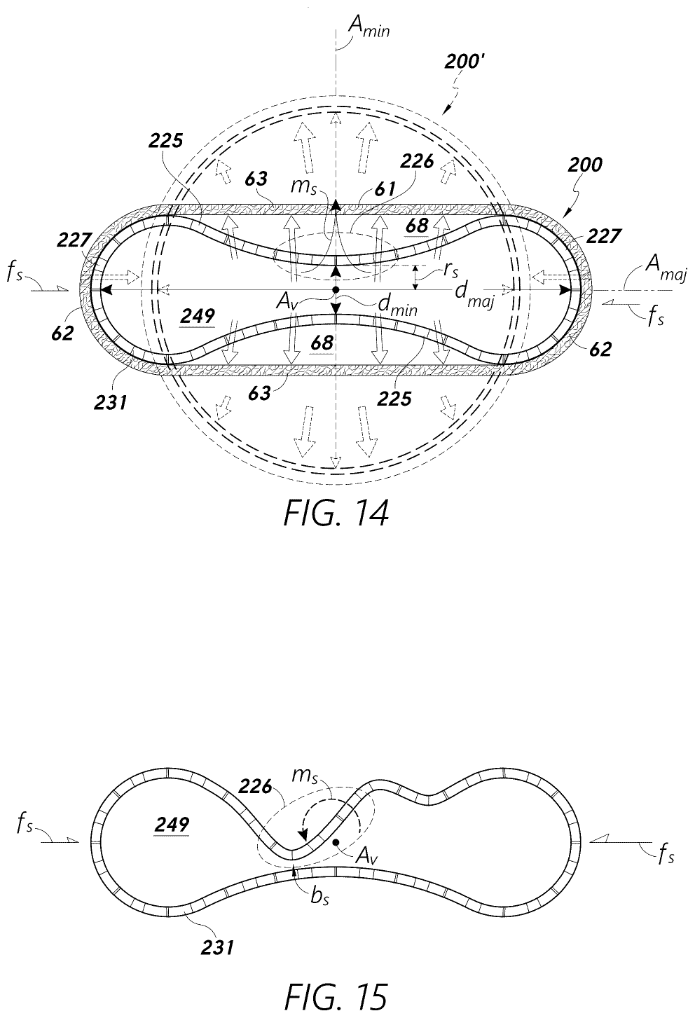

FIG. 14 shows an axial view of the uncovered, peanut-shaped stent 200 shown in FIGS. 13A and 13B deployed within a blood vessel 61 in accordance with one or more examples. The stent 200 is biased toward the illustrated peanut relaxed/diastolic configuration (shown in solid-line). When subjected to radially expansive forces, the stent is configured to responsively transform to a more circular systolic configuration (shown in dashed-line) such that the minor axis dₘᵢₙ approaches, and may equal, the major axis dₘₐⱼ. The cells 235 of the frame 231, formed by the arrangement of the struts 238, provide openings in the frame 231 that allow blood in the blood vessel in which the stent 200 is deployed to transfer pressure through the frame 231, to thereby load the inner diameter/surface of the blood vessel 61 with a force resulting from increases in the luminal blood pressure as the heart beats.

The blood pressure force against the blood vessel walls increases the hoop stress on the blood vessel 61, which may force the blood vessel, and with it the stent 200, to assume a more-circular shape. When the elasticity of the blood vessel wall 61 is compromised, the expansion of the blood vessel diameter is limited, and therefore, the pressure increase reshapes the blood vessel without substantially increasing the circumference thereof. The blood pressure force on the blood vessel wall 63 and resulting inward deflection of the blood vessel walls 62 at the major-axis-ends 227 of the stent 200 may cause inward deflection of the ends 227 of the stent 200 to form a desired geometric change to a more-circular shape of the stent 200, and with it the blood vessel 61.

The transition of the stent from oval to the more-circular stent shape 200′ (shown in dashed-line in FIG. 14) causes energy to be stored in the stent frame 231 (e.g., in the elasticity and/or shape memory thereof), such that energy is returned to the blood vessel walls 62, and therefore to the blood circulation within the blood vessel segment, when the frame transitions back to the oval shape as pressure decreases. The shape memory forces of the stent 200 are advantageously sufficient to overcome the pressure forces within the blood vessel 61 to return the stent 200 to the oval configuration in the presence of diastolic pressure conditions, thereby reshaping the blood vessel 61 to a non-circular (e.g., generally-oval) cross-sectional shape. With respect to implantation within the aorta or other arterial blood vessel, the systolic phase of the cardiac cycle, during which pressure levels in the aorta/arteries are relatively higher, causes the expansion of the blood vessel 61 and stent 200 to the more-circular shape (shown in dashed-line in FIG. 14), whereas the diastolic phase, which is associated with relatively lower arterial blood pressure levels, causes the elongation of the stent in the major axis Aₘₐⱼ dimension to the lower-energy oval configuration, thereby forcing the blood vessel 61 to likewise assume a more oval shape due to the blood vessel having a perimeter/circumference that is sufficiently close to the perimeter/circumference length of the stent 200 (e.g., within 20% of the length of the perimeter/circumference of the stent 200). As with any stent device disclosed herein, the stent 200 can be implanted within the venous system, such as within the inferior vena cava or superior vena cava.

The natural cross-sectional shape of the aorta (and other blood vessels) may generally be circular; as explained above, for a given blood vessel wall circumference/perimeter length, the circular configuration of the blood vessel may provide the maximum area/volume within the respective blood vessel segment. Therefore, any deviation from such circular/cylindrical form of the blood vessel wall may decrease the area/volume within the respective blood vessel segment. With the oval stent 200 pushing outward to the oval configuration, the wall portions 63 may be pulled/drawn at least partially towards an axial center Aᵥ of the blood vessel 61 and/or towards each other in a manner as to cause the blood vessel 61 to form a non-circular/-cylindrical shape, such as the peanut shape shown in FIG. 14. That is, compared to the circular/cylindrical shape of the blood vessel 61. the blood vessel 61 in the oval shape has a cross-sectional area in the axial segment/area where the stent 200 is implanted that is reduced.

Due to the circumference/perimeter of the blood vessel 61 being similar to the circumference/perimeter of the stent frame 231, the configuration of the stent 231 in the oval shape causes the or aortic wall 61 to assume a more oval or peanut shape to match the stent 200. However, depending on the relative size of the stent 200 to the vessel 61, the blood vessel 61 may not necessarily conform exactly to the circumference and/or shape of the oval stent 200, and some minor gap 68 between the stent walls 225 and the vessel walls 63 may be present and/or form as the luminal pressure increases and pushes the vessel side walls 63 away from the frame sidewalls 225.

As the stent 200 comprises a bare-frame stent, wherein the frame 231 is not covered by a fluid-tight covering, the origin of the driving force required to transition the frame 231 from the oval shape to the more circular shape 200′ may necessarily be based on hoop pressure/stress against the vessel inner diameter, wherein the luminal pressure exerts radial outward force against the vessel internal wall, such forces indirectly acting against the major axis dₘₐⱼ of the stent 200 to force outward deflection of the sidewalls 225 towards the circular shape 200′. For example, as the blood pressure increases in the vessel 61, the hoop stress on the blood vessel walls may force the side wall portions 63 of the blood vessel 61 to deflected radially outward towards a more uniform circular shape of the blood vessel 61, wherein the stiffness of the blood vessel wall causes the outward deflection of the sidewalls 63 to pull radially inwardly on the end walls 62 of the blood vessel, thereby applying radially-inward pressure on the end walls 227 of the stent frame 231. Such pressures force inward deflection of the walls 227 of the stent 200, thereby allowing the sidewalls 63 of the blood vessel 61 to deflect outwardly towards the circle dimension.

The translation of force from the end wall 227 to the side wall 225 can cause a radially-outward moment/force in the side wall 225. For example, the force vector pushing radially inwardly on the side walls 227 can create peripheral tension/stress (e.g., ring/hoop stress) in the sidewalls 225 due to the mechanical continuity/coupling between the end walls 227 and the sidewalls 225, which can cause the outward deflection of the sidewalls 225. Therefore, with a bare-frame peanut stent, transition force for transitioning the stent to a more-circular shape from a peanut shape may necessarily be implemented from two primary contact points in the area of the sidewalls 227 and/or vertices vₑ associated with the major-access ends of the stent frame 231. Such forces inwardly compress the major axis dₘₐⱼ of the stent 200. The force vectors pushing against the ends 227 of the frame 231 may necessarily be required to be sufficient to overcome the concentrated resistance at the sides 227 and/or vertices Vₑ of the frame 231. In view of the elongated shape of the peanut frame 231, the radially-outward resistive force of the frame 231 may be greater at the major access ends 227 than at the minor axis walls 225, such that a relatively substantial force may be required to cause the inward deflection of the frame ends 227.

As the pressure in the blood vessel 61 increases (e.g., in connection with the systolic phase of the cardiac cycle), the plastically-deformable nature of the stent 200 allows for the sidewalls 225 of the frame 231 to be pushed outward to accommodate the shortening of the stent 200 in the major axis dimension Aₘₐⱼ. When the ends 227 of the stent 200 are brought closer together (as pushed radially inward by the blood vessel walls 22, the stent 200 allows the blood vessel 61 to assume a more circular cross-sectional shape. That is, the stent 200 improves cardiac perfusion by causing a decrease in cross-sectional blood vessel area during diastole relative to systole due to the reshaping of the blood vessel caused by the lengthening of the major diameter dₘₐⱼ of the stent 200.

As shown in FIG. 14, for an uncovered peanut-shaped stent, during high pressure/force conditions (e.g., systole), a concentrated force fₛ is imparted/exerted on the stent 200 at its contact points with the blood vessel walls 62. Such force may be translated to the concave/waist portion 226 of one or both of the sidewalls 225 with a magnitude that is a product of the distance rₛ between the concave portion 226 and the center of the stent 200 with respect to the minor axis Aₘᵢₙ when the force is exerted. Ideally, the force fₛ is translated from the sidewalls 227 to the concave/central portion 226 of the sidewalls 225 in a manner as to outwardly deflect the sidewalls 225. The translation of force from the end walls 227 to the concave portion 226 of the sidewalls 225 can produce a force moment mₛ. The moment mₛ, depending on the magnitudes of the forces fₛ from the sides 227 and/or the relationship or relative magnitudes thereof can result in a rotational force/moment having clockwise and/or counterclockwise component(s) with respect to the axial view shown. Where the forces fₛ are substantially balanced, the force moment MS may advantageously push the concave portion 226 outward in a manner as to move towards the more-circular shape 200′. That is, ideally, the concave portions 226 of the sidewalls 227 are deflected in an amount and/or shape such that when force is applied from the side walls 227, based on the cell structure and/or other characteristics of the frame 231, the walls 225 are pushed out towards the more-circular shape 200′ desired, rather than buckling inward and/or in the direction of the major axis Aₘₐⱼ.

FIG. 15 shows the peanut-shaped stent 200 experiencing buckling in accordance with one or more examples. Where the design of the stent 200 is such that the moment mₛ produced by the inward force(s) fₛ from one or more sides of the frame 231 causes a rotational moment having, for example, counterclockwise (or clockwise) chirality, buckling bₛ may occur, which may prevent the stent 200 from achieving the desired circular shape as pressure/force increases, but rather may cause the frame 231 to crimp, fold, bend, and/or otherwise buckle in a manner as to prevent the desired outward deflection of the wall(s) 225.

Various parameters or factors associated with the design of the stent 200 may contribute to the tendency thereof to buckle in a manner shown in FIG. 15. For example, the stent strut 238 shape and/or geometry may affect the buckling tendency thereof. Furthermore, the degree to which the concave sidewalls 225 deflect inward toward the axis Aₛ of the stent 200 and/or towards the major axis Aₘₐⱼ. When buckling occurs, the walls 225 may deflect inward rather than outward, thereby defeating the compliance-enhancing functionality of the stent 200.

Generally, it may be desirable to design the stent 200 to be slightly oversized relative to the size of the target blood vessel to prevent axial migration of the stent when deployed in the blood vessel. If the stent is designed too large relative to the blood vessel, such oversizing of the stent 200 can compound the risk of buckling occurring by forcing the sidewalls 225 to deflect to a greater degree inward than desired. Such oversizing of the stent can cause the stent frame 231 to form a figure-eight shape, wherein the walls 225 are brought substantially close to one another, rather than the desired peanut shape illustrated in certain figures herein. In some implementations, the patient anatomy may be determined through imaging and/or other visualization/characterization thereof, which may be relied upon for the purpose of determining a diameter of the target blood vessel. Therefore, the design of the stent for the particular patient anatomy may be impactful with respect to the risk of buckling.

Buckling risk may be reduced with respect to peanut-shaped stents that include covering(s), as described in detail herein. For example, such coverings may advantageously spread pressure forces on an inside of the deflected/concave minor-axis sidewalls of the stent, thereby preventing such walls from buckling inward. Therefore, stents having concave/inwardly-deflected sidewalls may derive particular benefit from being implemented with stent coverings in accordance with aspects of the present disclosure.

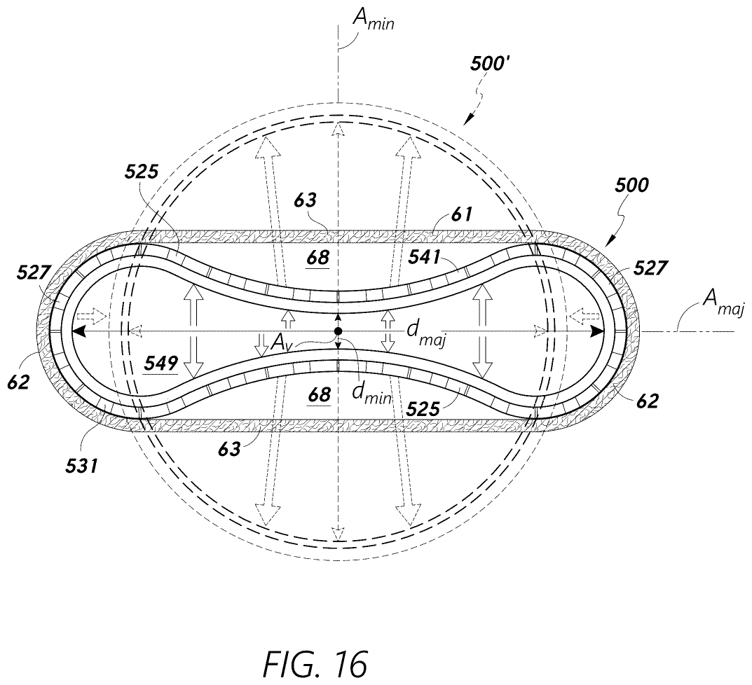

FIG. 16 shows a covered peanut-shaped stent 500 deployed within a blood vessel 61 in accordance with one or more examples. With respect to the bare-frame peanut stent 200 (e.g., without a fluid-tight covering) shown in FIG. 14, the mechanism required for transitioning an implanted peanut-shaped oval stent may involve the inward contact/pressure of the blood vessel wall along the major axis dimension Aₘₐⱼ against the major-axis end walls 227, which may indirectly result in the radially-outward deflection of the concave sidewalls 225, ultimately resulting in a more circular shape of the frame if undesirable buckling does not occur. Such indirect manipulation of the sidewalls may be relatively less efficient with respect to the forces required, and/or resulting shape produced, when transitioning the stent from the peanut oval shape shown to a circular shape. Furthermore, indirect forcing of the deflection of concave sidewalls can result in buckling that defeats the ability of the stent to form a circular shape. Therefore, covering the stent frame on inner and/or outer surfaces thereof can be beneficial to allow for more even distribution of hoop forces against the stent frame to prevent bucking and more efficiently and/or effectively transition the frame from the peanut shape to a circular, or more-circular, shape.

In the peanut stent 500 configuration of FIG. 16, the longer/flatter sidewalls 525 of the frame 531, which may comprise sides/walls of the frame 531 that run generally in alignment with and/or parallel to the long/major axis Amy of the stent 500, may be deflected inward in an externally concave manner, as shown, such that the diameter between the walls 525 is greater in the major-axis end regions than in the medial region in the area of the minor/short axis Aₘᵢₙ.

Adding a cover 541 to the stent 500 can mitigate the risk of buckling by spreading the blood pressure forces along the inner wall of the stent 500, such that substantially the entire perimeter thereof is pressed radially outwardly. Such outward pressure/force can resist any force/moment inclined to otherwise torque the waist 526 of the frame inward on one or more sides thereof to cause buckling.

In the presence of inwardly-directed forces along the major axis Aₘₐⱼ on the respective sidewalls 527 of the frame 531, the concavity/deflection of the sidewalls 525 may be resolved, and further outward deflection/external-convexity may occur in the walls 525, such as in the area of the minor axis Aₘᵢₙ.

Once deployed within a blood vessel, the stent 500 (and blood vessel). during diastolic pressure, may remain in a somewhat peanut shape or, due to outward pressure from the blood within the blood vessel (even during diastole), the stent 500 and/or blood vessel may deform into a substantially oval, non-concave/peanut shape. When subjected to the larger pressures of systole, the stent 500 and/or blood vessel may deform to a more circular shape. The covering 541 may serve to facilitate such reshaping of the stent 500 to achieve the desired change in an area/volume of the stent throughout the cardiac cycle.

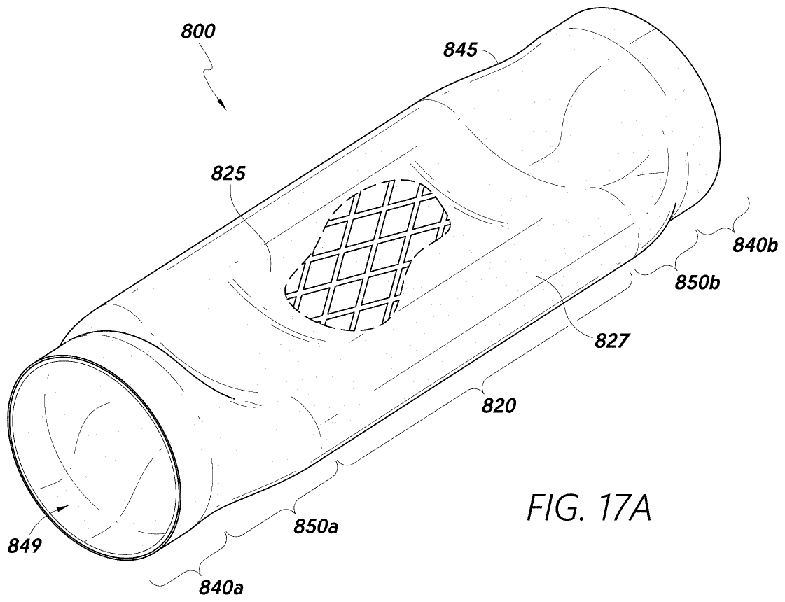

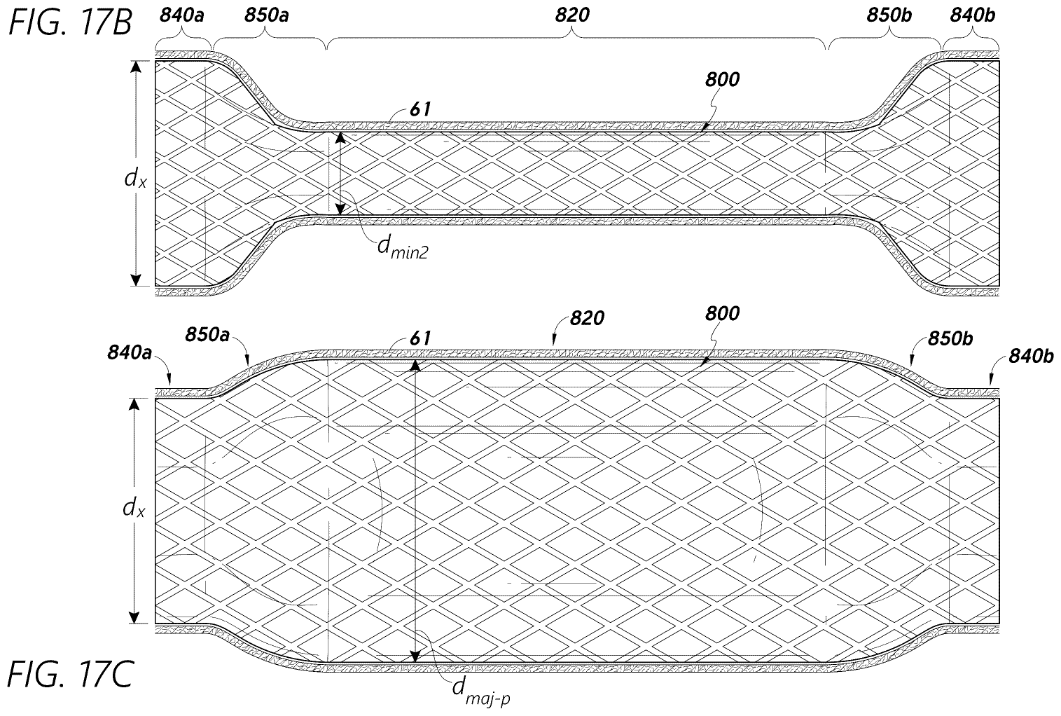

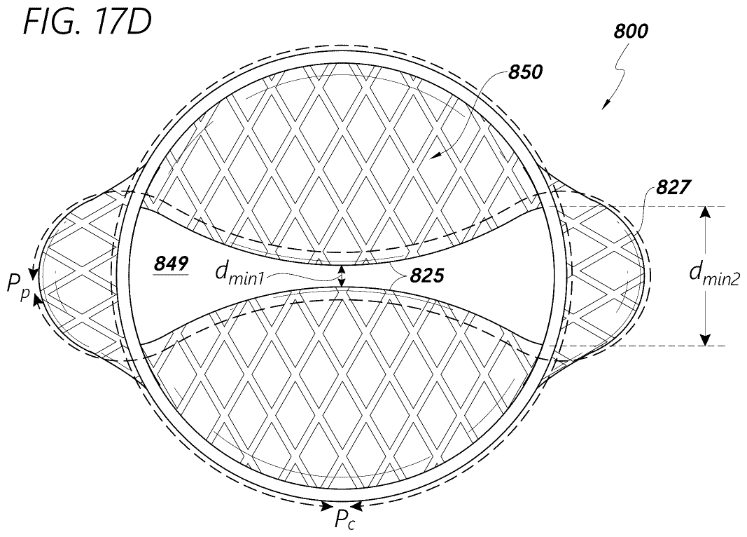

FIGS. 17A-17D show perspective, side, and axial views, respectively, of a stent 800 having circular end portions 840 and a non-circular (e.g., peanut-shaped) medial portion 820 in accordance with one or more examples. Although the device 800 is described below as a stent, it should be understood that, as with any other device disclosed herein, the device 800 may be implemented and/or implanted as a graft that replaces a resected blood vessel segment or other gap between blood vessel segments. For example, the end portions 840 of the devices 800 may be sutured or otherwise attached to open ends or openings of/in blood vessel segment(s), such that at least a portion of the length of the medial portion 820 of the device 800 spans a distance between the blood vessel segments and/or is not entirely endovascular.

The stent 800 may have any of the features disclosed above in connection with the stent 400 of FIGS. 12A-12D. However, with respect to the medial portion 820 of the stent 800, such portion may have a cross-sectional axial shape resembling the peanut-type shape disclosed in FIGS. 13A and 13B, as well as in other figures of the present disclosure, which are described in detail herein. Similarly to the stent 400 of FIGS. 12A-12D, the stent 800 can include axial circular ends 840 and transition portions/segments 850 that transition the shape of the stent 800 between the circular ends 840 and the peanut-shaped medial portion 820.

The medial portion 820 may have any suitable or desirable length, and may comprise inwardly-deflected minor axis walls 825, which are connected at major axis ends 827 as with other examples disclosed herein. Although a particular peanut shape is shown for the medial section 820, as illustrated clearly in the axial view of FIG. 17D, it should be understood that the medial segment/portion 820 may have any suitable or desirable degree of deflection/curvature, or other features defining an at least partially peanut-shaped conduit. For example, the medial portion 820 may have any suitable minimum diameter dₘᵢₙ₁ (see FIG. 17D) and/or any suitable or desirable maximum minor-axis dimension dₘᵢₙ₂. Furthermore, the curvature and/or angle(s) of deflection of the walls 825/827 of the peanut-shaped shaped medial portion 820 may have any suitable or desirable parameters. As shown in FIG. 17A, the stent 800, as with other examples disclosed herein, may or may not comprise a covering 845, which may be disposed on an outer surface and/or inner surface of a frame/skeleton structure of the stent 800. The stent frame 800 can comprise a solid-surface form, rather than a structure formed of struts and open cells as illustrated and described in detail herein.

From the top view of FIG. 17C, it can be seen that in some examples, the peanut-shaped medial portion 820 has a major diameter dₘₐⱼ₋ₚ that may be greater than a diameter dₓ of the circular portion 840. Conversely, the minor axis dimension dₘᵢₙ₂, as shown in the side view of FIG. 17B, may have a value less than the diameter dₓ of the circular portions 840. As with other similar examples disclosed herein, the circular end portions 840 may be configured to provide a desirable (e.g., hemostatic) seal between the axial end(s) of the stent 800 and the surrounding blood vessel wall, whereas the differently-shaped medial portion 820 may be shaped to cause radial elongation of the blood vessel in which it is implanted, to thereby transition the blood vessel between oval-shaped and more-circular-shaped in the presence of relatively high luminal pressure within the channel 849 of the device 800. That is, in high pressure conditions, the stent 800 may assume a substantially-continuously circular/cylindrical shape throughout the axial length thereof.

In some examples, the perimeter/circumference P_{c} of the circular portions 840 may be substantially equal to the perimeter/circumference Pₚ of the peanut-shaped portion 820 and/or at least a portion thereof. Therefore, when the peanut-shaped portion 820 transitions to a more-circular shape, the cross-sectional shape thereof may resemble that of the circular ends 840.

FIGS. 12A-12D and 17A-17D, described in detail above, relate to non-circular stents having circular end portions/edges, which provide sealing and/or funneling functionality. Generally, such circular stent end portions may not contribute significantly to the compliance or efficacy of the device with respect to increasing diastolic flow and/or limiting systolic flow through the stent. Therefore, such end segments can be considered to be occupying space that otherwise could be dedicated to compliance-enhancing elements/segments. Furthermore, circular stent ends/edges can act as a stationary harness that at least partially limits the movement of the non-circular middle/medial portion (e.g., peanut-shaped portion, as in FIG. 17A-17D). Furthermore, when moving from circular end portions into a peanut-shape (or oval-shaped) medial portion, such transition may represent relatively drastic narrowing that may cause flow issues and/or manufacturing complications with respect to relatively large deformation/strains.

With particular reference to the embodiment of FIGS. 17A-17D described in detail above, which relates to a covered peanut-shaped stent 800 including circularly-shaped ends 840 that seal against the surrounding vascular wall, the non-circular central/medial portion of the stent disposed between the circular ends can be configured to be biased toward the peanut-shaped configuration under low pressures. Under relatively-higher systolic pressure conditions, the aorta (or other blood vessel in which the stent is implanted) can be deformed into a more-circular shape, after which the stent is configured to revert the aorta back toward the peanut shape in the area spanned by the peanut-shaped medial portion 820. In this manner, the peanut-shaped stent segment is configured to restore some compliance to the otherwise non-compliant (or less-complaint) aortic section. Since the circular end portions 840 are not configured to change in shape, they generally may not contribute to the compliance or efficacy of the stent 800, and may even limit the transitional movement of the stent's central/medial portion 820. Moreover, where the stent 800 presents a relatively abrupt transition from the circular to the narrower (e.g., significantly-narrower) peanut-shape can result in flow disturbances, and may present manufacturing complication associated with significant deformations/strains.

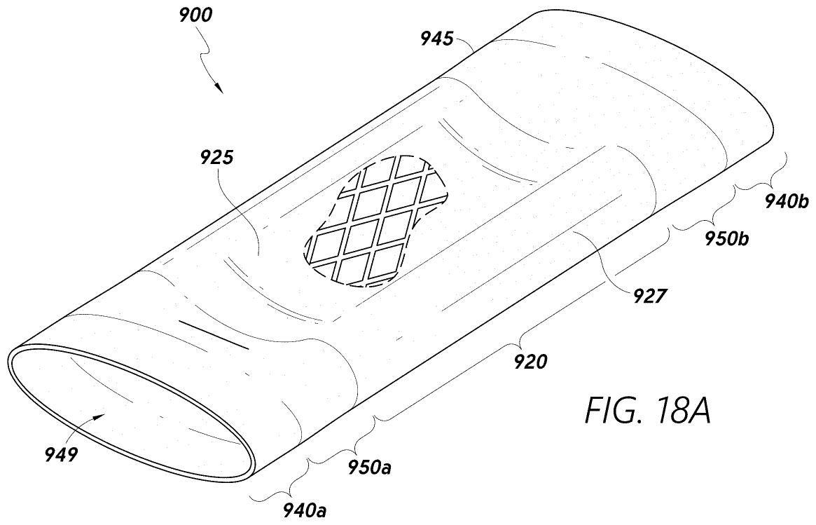

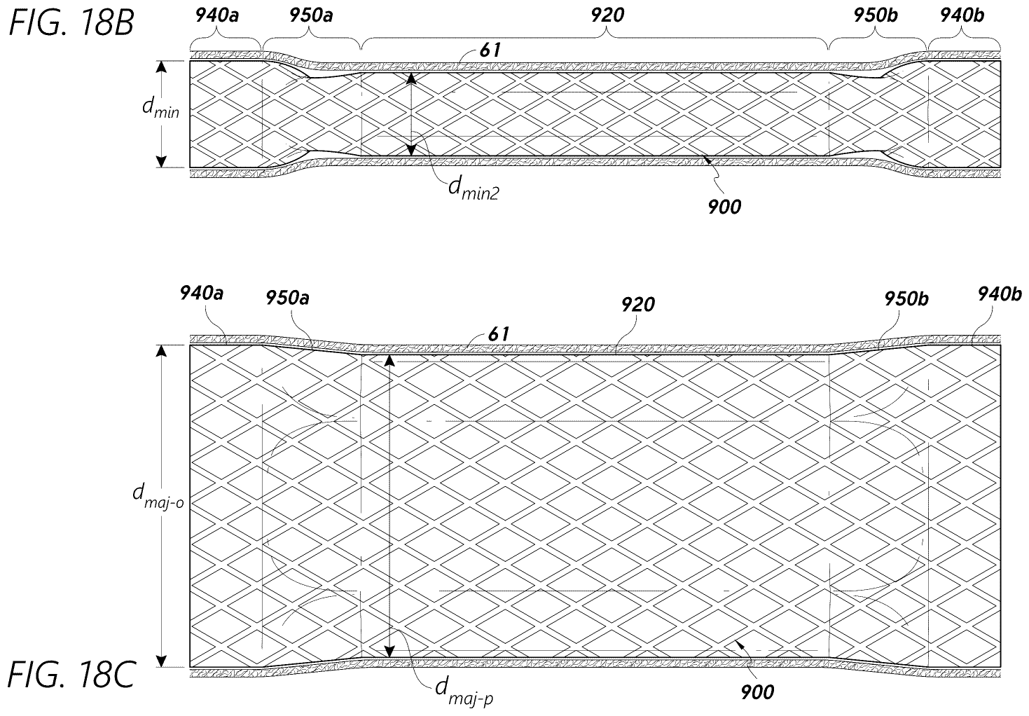

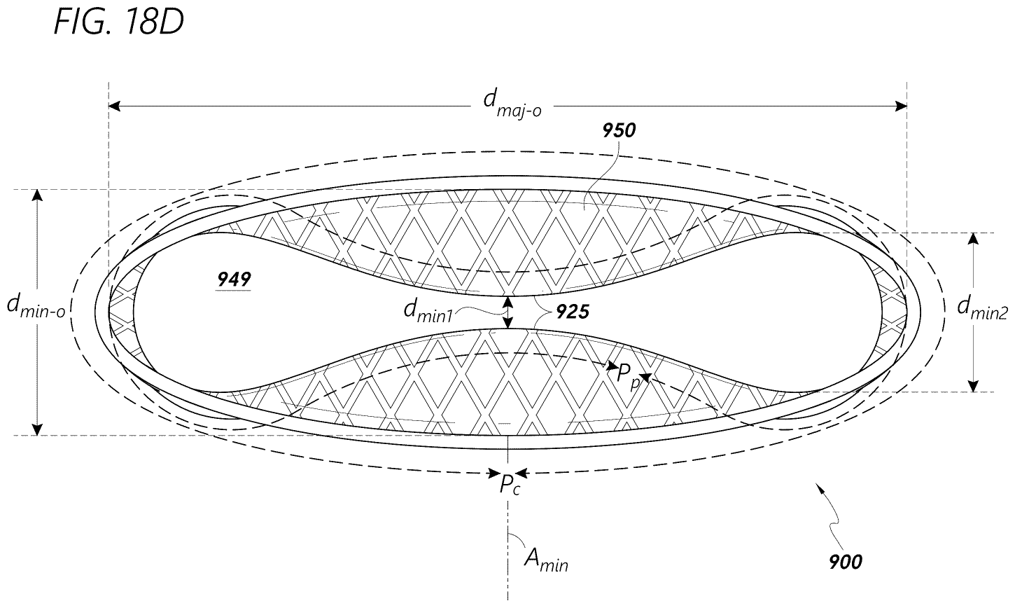

In some implementations, the present disclosure relates to variations of stent designs having differently-shaped end and medial portions/segments. For example. aspects of the present disclosure can provide stents having end portions that are not circular, but rather some non-circular shape that is different than the non-circular shape (e.g., peanut shape) of the medial portion of the stent. In such examples, one or both ends of a compliance-enhancing stent can be designed, with respect to a relaxed shape/configuration thereof, as compliant oval-shaped portions, which may advantageously retain proper sealing against the blood vessel, while also contributing to the stent's blood-vessel-reshaping functioning during transitions between low (e.g., diastole) and high (e.g., systole) pressure phases/conditions. Such stent end shaped can further form more gradual transitions in shape that reduce the likelihood of flow disturbances through the stent. FIGS. 18A-18D show perspective, side, and axial views, respectively, of a stent 900 having oval end portions 940 with radially-outwardly-deflecting/bowing minor-axis sidewalls and an oval medial portion 920 with radially-inwardly-deflecting/concave minor-axis sidewalls (e.g., peanut shape), in accordance with one or more examples. Although the device 900 is described below as a stent, it should be understood that, as with any other device disclosed herein, the device 900 may be implemented and/or implanted as a graft that replaces a resected blood vessel segment or other gap between blood vessel segments. For example, the end portions 940 of the devices 900 may be sutured or otherwise attached to open ends or openings of/in blood vessel segment(s), such that at least a portion of the length of the medial portion 920 of the device 900 spans a distance between the blood vessel segments and/or is not entirely endovascular when implanted.

The stent shape of the stent 900 of FIGS. 18A-18D may have certain advantages over other stent designs with a peanut-shaped medial portion and including non-compliant circular end portions. For example, while for some designs with circular end portions, such end portions may not be configured to change in shape throughout the cardiac cycle, and thus may not contribute to the stent's compliance-enhancing vessel-reshaping functionality, the oval end portions 940 of the stent 900 may be configured to change in shape in a cyclical manner, thereby contributing to compliance enhancement. Furthermore, while circle-shaped end portions may limit the transitional movement of a stent's central/medial portion, the oval-shaped end portions 940 may be less limiting with respect to the transitional movement of the medial, peanut-shaped portion 920 between the relaxed peanut shape thereof and a more-circular configuration. In addition, while some designs with circular end portions may present relatively abrupt transitions from the circular shaped ends to the narrower (e.g., significantly narrower) peanut-shape of the medial portion of the stent, which can result in flow disturbances, and may present manufacturing complication associated with significant deformations/strains, the transition between the oval shape of the end portions 940 and the peanut shape of the medial portion 920 may have less of an effect on the flow and structural design strains/deformations than some circle-end stents.

The stent 900 may be similar to either or both of the stent 400 and 800 described above in connection with FIGS. 12A-12D and FIGS. 17A-17D, respectively, with the exception that the end portions 940 of the stent 900 may have an oval or other non-circular cross-sectional/axial shape in a relaxed state thereof, as opposed to the circular end portions described above in connection with other examples.

The oval end portions 940 can transition to a peanut-shaped medial portion/segment 820, wherein transition segments/portions 950 may transition the various dimensions/diameters of the oval segments 940 to those of the peanut-shaped segment 920. The configuration illustrated in FIGS. 18A-18D represent the stent 900 in a relaxed, low-pressure configuration, wherein higher luminal pressure in the channel 949 of the stent 900 may cause the end portions 940 and/or the peanut-shaped medial portion 920 to expand in the minor axis dimension Aₘᵢₙ to move the shape of the end portions 940 and the shape of the medial portion 920 to more-circular shapes. Therefore, with respect to the example shown in FIGS. 18A-18D, substantially the entire length of the stent 900 may transition between non-circular and circular cross-sectional shapes throughout the cardiac cycle, thereby potentially increasing the amount of total volume change for the stent 900 relative to its overall length, which may increase compliance-enhancement functionality thereof. The perimeter/circumference of the oval end portions/segments 940 may be similar to the perimeter/circumference of the peanut-shaped portion/segment 820. Therefore, in the presence of sufficiently-high luminal pressures, the end 940 and medial 920 segments may assume relatively circular cross-sectional shape that is substantially continuous along the length of the stent 900. As shown in FIG. 18A, the stent 900, as with other examples disclosed herein, may or may not comprise a covering 945, which may be disposed on an outer surface and/or inner surface of a frame/skeleton structure of the stent 900.

Advantageously, the oval shape of the end portions 940 can serve to retain contact along its entire circumference with the blood vessel (as with circular end portions of examples disclosed herein), thus preserving proper scaling against the artery wall and channeling the flow into the stent's lumen through the medial portion 92. Sealing can be achieved with the oval-shaped end portions 940 due to the contact of the oval end portions along substantially the entire circumference thereof, unlike peanut-shaped stent portions, which may have a tendency to present gaps on minor-axis sidewalls and/or pull such walls away from the blood vessel wall in concave areas of the stent wall.

Furthermore, since the oval end portions are compliant due to the reshaping ability thereof with respect to their ability to transition between oval and circular configurations between the diastolic and systolic phases, the end portions 940 can contribute to the effectiveness and functioning of the stent as a flow-improving implant device. The end portions 940 can transition in their cross-sectional shape along with the central/medial peanut-shaped portion 920, thus avoiding the disadvantages of examples of stents with circular end portions that potentially limit the transitional movement of the medial portion thereof. Since the edges/ends 940 are generally not stationary, such portions can present as a less drastic transition from the peanut-shaped portion 920,

In addition, the transition between the oval end portions 940 and the central/medial peanut-shaped portion 920 may advantageously be more gradual than certain other example devices, thus reducing the likelihood of flow disturbances as blood flow moves through the stent. The design of the stent 900 provides relatively less drastic narrowing in the stent, and more gradual narrowing in the vessel naturally.

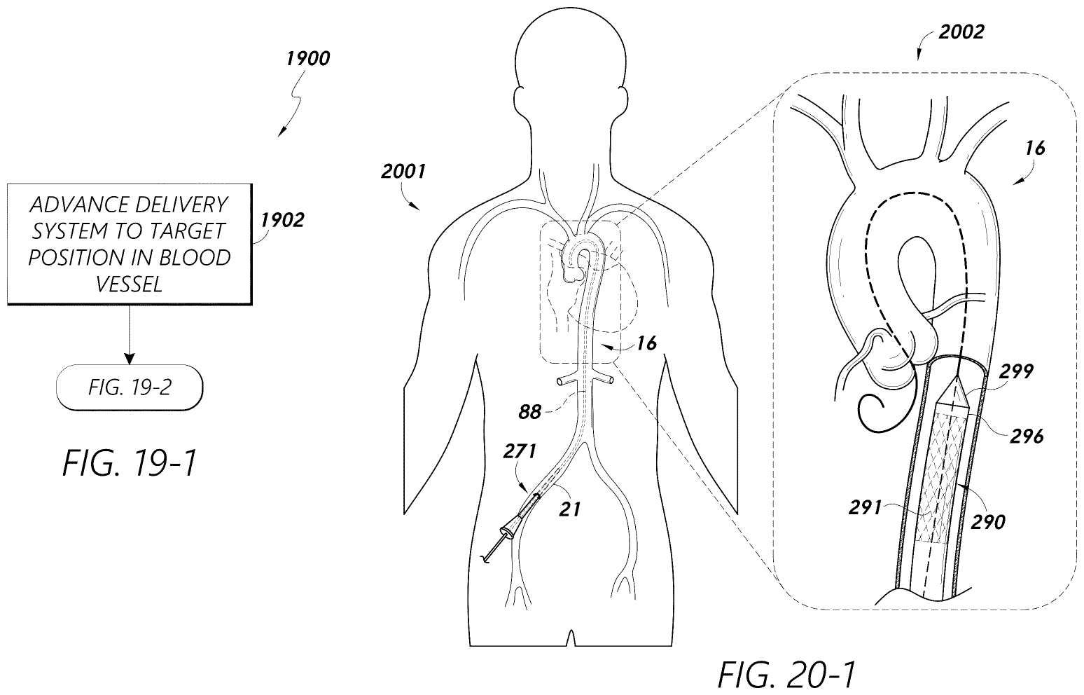

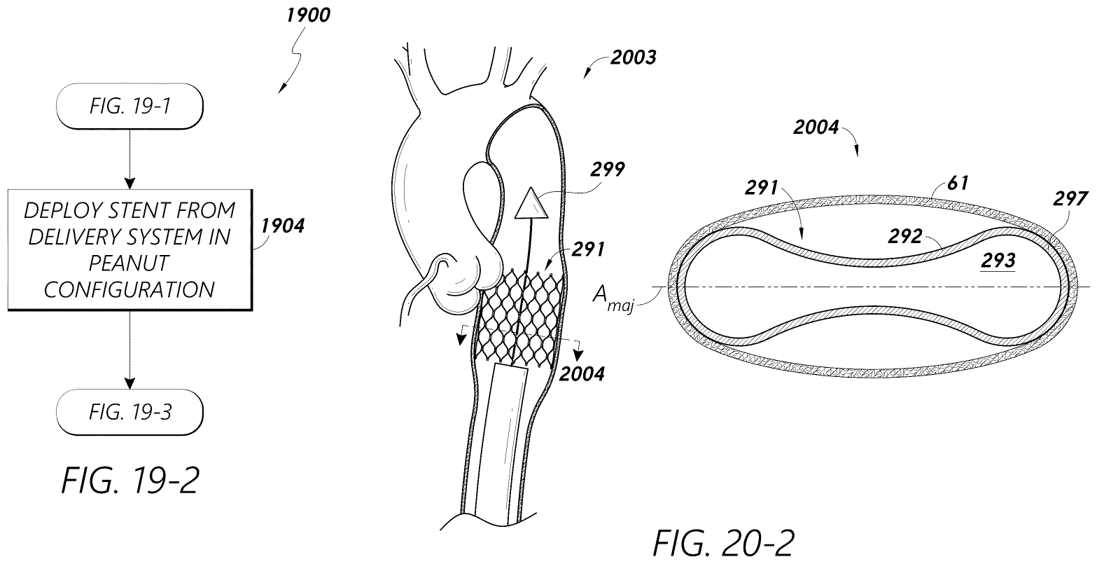

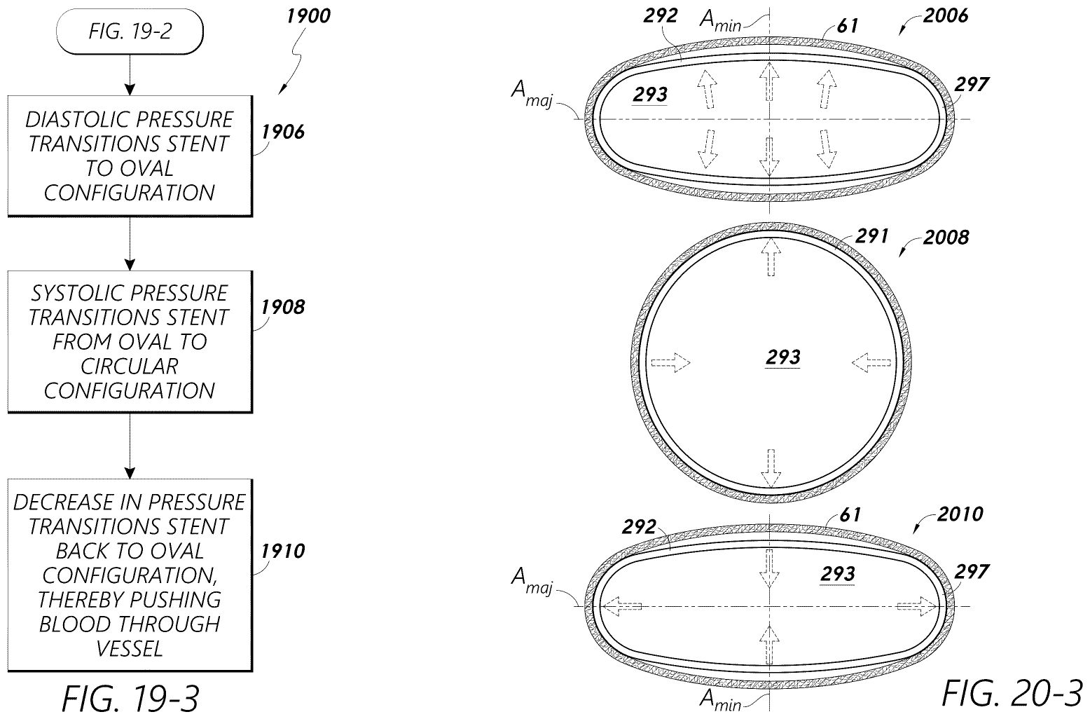

FIGS. 19-1, 19-2 and 19-3 illustrate a flow diagram for a process 1900 for reshaping a blood vessel using a non-circular stent 291 in accordance with one or more examples. FIGS. 20-1, 20-2, and 20-3 provide images of the stent 291 and certain anatomy corresponding to operations of the process 1900 of FIGS. 19-1, 19-2, and 19-3 in accordance with one or more examples.

At block 1902, the process 1900 involves advancing a delivery system 290 to a target position in a blood vessel 16, such as the aorta, as shown in image 2001. For example, the delivery system 290 may be advanced through a percutaneous introducer or other minimally-invasive access 271 into the vasculature of the patient, and further within the vasculature to a target position within the aorta 16 of the patient. The delivery system may include one or more catheters/sheaths 296 and/or a nosecone 299 or other feature configured to facilitate the forward advancement of the delivery system 290 through tortuous anatomy of the vasculature.

At block 1904, the process 1900 involves deploying a non-circular stent 291 from the delivery system 290, as shown in image 2003, wherein the stent 291 may be transported to the target site in a radially crimped configuration (see image 2002) to fit within a catheter/sheath 296 having relatively small profile. The stent 291 may have a relaxed shape that resembles a peanut or other non-circular shape, wherein the axial cross-sectional form of the stent 291 (see image 2004) includes long sidewalls 292 that are deflected radially inward to some degree, as described in detail herein.

At block 1906, the process 1900 involves submitting the stent 291 to the minimum pressure levels in the blood vessel 16, which may correlate with diastolic pressure levels, for example. In such pressure conditions, the stent 291 may be configured to assume a transitional configuration/form as shown in image 2006 between the peanut configuration shown in image 2004 and the circular configuration shown in image 2008. That is, while the stent 291 may be configured to assume a peanut-shaped form while in a relaxed state in the absence of internal luminal pressure forces, it should be understood that such non-circular stents may have such peanut shape/configurations in a free-relaxed state out of the patient's body, but may be inclined to assume a more-oval shape as shown in image 2006 (e.g., involving the straightening of the longer sidewalls 292 that run generally in the orientation of the major axis Aₘₐⱼ of the stent 291) when subjected to diastolic pressure in the patient's body. Therefore, in connection with on-going reshaping/transitioning over cardiac cycling, the stent 291 may transition between the diastolic oval shape of image 2006 (rather than the peanut shape of image 2004) and the more-circular shape of image 2008 during systole.

The shape of the stent 291 in the oval transition shown in image 2006 can resemble an oval shape, wherein the sidewalls 292 are not deflected inwardly as in the image 2004, but rather are substantially straight, or slightly outwardly-deflected/bowed, and/or remain inwardly deflected albeit to a lesser degree than in the image 2004. Therefore, by constructing the stent 291 in such manner as to have the sidewalls 292 inwardly-deflected, as opposed to straight or outwardly deflected as with some oval stent examples, the minimum pressure conditions within the target blood vessel may serve only to expand the stent to an oval shape, rather than pushing the stent walls 292 towards a more-circular configuration. Therefore, in such state, the volume change between diastolic and systolic phases of the cardiac cycle may be maximized compared to configurations in which diastolic pressure conditions force the stent into more-circular configurations, such that the shape transition between diastolic and systolic phases is less substantial than with the peanut-shaped examples. Furthermore, in implementations in which the stent 291 is covered internally and/or externally, as described in detail herein, risk of buckling at the waist of the peanut shape of the stent may be mitigated at least in part by spreading the expansive pressure forces along the inner wall of the covering and/or stent frame, and therefore along the perimeter of the stent, thereby pushing radially outward against the inwardly-deflected walls 292 in a manner as to prevent inward bending/contortion that may otherwise result in buckling.

At block 1908, the process 1900 involves exposing the channel 293 of the stent 291 to blood pressure levels within the blood vessel lumen that are associated with a relatively high-pressure phase of the cardiac cycle, such as the systolic phase of the cardiac cycle with respect to arterial implantation. In such conditions, the stent 291 may further expand to a more-circular, fully-expanded configuration, as shown in image 2008.

At block 1910, the process 1900 involves submitting the stent 291 to decreased pressure levels as the blood pressure transitions back from higher-pressure to lower-pressure moving from, for example, systole to diastole. Such decrease in pressure may allow the shape-memory of the stent 292 to transition back to the oval configuration thereof (see image 2006), although the stent 291 may not revert fully to the original peanut-shaped configuration (see image 2004) due to the pressure in the channel 293 maintaining a minimum pressure associated with the circulation, such pressure being sufficient to hold the walls 292 in the outwardly-bowed oval configuration of image 2006.

Preliminary peanut-to-oval transition/reshaping of the stent 291 in the presence of minimum blood pressure levels in the aorta or other blood vessel (see block 1906 and image 2006) can advantageously improve the long-term durability of the stent 291, as the transition between concave and convex configurations of the minor-axis sidewalls 292 of the stent 291 can subject the walls to relatively high levels of fatigue compared with the transition between the less-concave (e.g., linear), or convex, oval configuration of the walls 292 shown in image 2006 and the high-pressure circular configuration shown in image 2008.

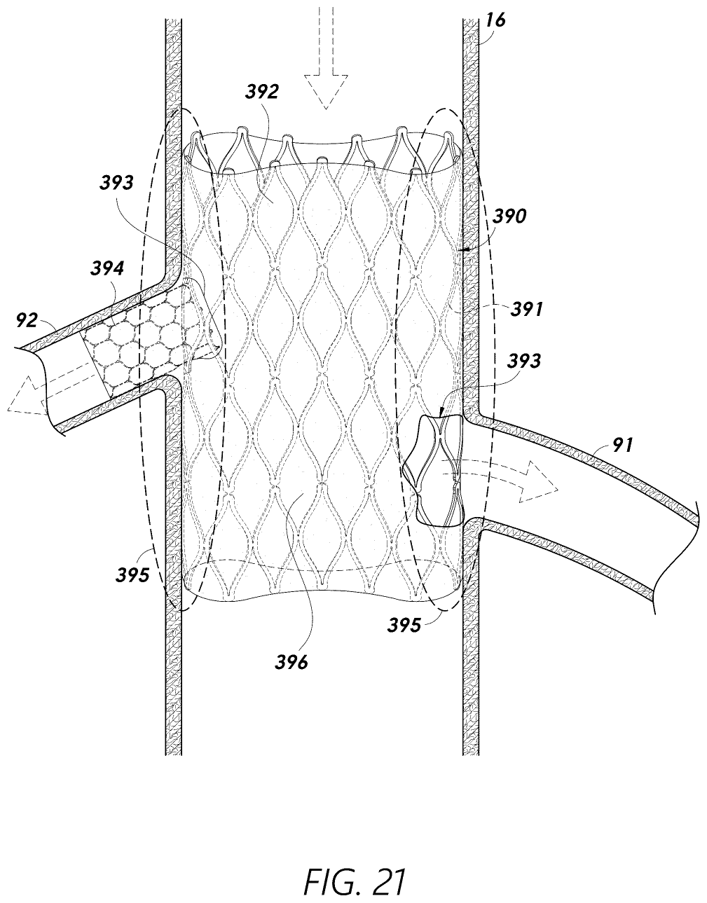

FIG. 21 shows a covered non-circular stent 390 having one or more fenestrations/openings 393 in a covering 392 thereof accordance with one or more examples. The stent 391 may represent an example implementation of any of the non-circular stents disclosed herein, wherein such stents include a covering to promote the oval/non-circular-to-circular transition of the frame/structure thereof in response to increases in luminal pressure. The stent 390 includes a covering 392, which may be disposed on an exterior or interior of the frame 391 of the stent 390. The stent frame 391 may have an oval and/or peanut-shaped form, in accordance with any of the examples disclosed herein. Furthermore, the covering 392 may be disposed on an inside surface/portion of the stent frame 391, on an outside thereof, or both inside and outside of the frame 391. The target blood vessel 16 (e.g., thoracic or abdominal aorta) may have certain side branches 91, 92, which may comprise intercostal blood vessels, vertebral blood vessels, or the like, and may project from right and/or left sides of the blood vessel 16, as shown.

The covering 392 includes one or more openings or fenestrations 393 therein, which may be positioned to accommodate blood flow through the covering 392 into one or more side branches 91, 92 associated with the target blood vessel 16. The positioning of the opening(s)/fenestration(s) 393 may allow for implantation of a covered non-circular/oval stent 391 in accordance with aspects of the present disclosure, wherein the covering 392 does not occlude/block blood flow into the side branch(es) from the target blood vessel 16, which may otherwise result in reduced supply of blood to organs or tissues associated with such branches, which may be detrimental to the health of the patient.

By implementing covering fenestrations 393 as shown in FIG. 21, the blocking of flow into side branches that may exist at the implantation site can be avoided, thereby allowing for continuous perfusion of such branches. Continuous perfusion of side branches may further be facilitated by implementing parallel stent grafting via chimney/periscope stent frame configurations that are designed to fit within side branch(es). For example, the stent 390 may include the illustrate side stent 394 emanating from and/or otherwise associated with the stent frame 391. The branch stent 394 may shoot off of the main body of the stent 390 and extend into one or more side branches 92 to thereby hold the lumen and/or ostium thereof in a desirable open state and/or further secure the stent 390 in place, with cover fenestrations 939 properly positioned over the side branch(es) to permit flow through the covering 392. That is, side branches may be accommodated by fenestrations 393 in the covering 392 and/or by additional stent frame/structure(s) 394 configured to engage therewith and channel blood into such branches.

Covering fenestrations and/or auxiliary branch stent structures in accordance with aspects of the present disclosure can help to keep critical branch blood vessels irrigated. It may be desirable to design such stents in a manner as to keep the fenestrations thereof stationary after implantation in relation to the side branch vessel ostium associated with each fenestration.

As described in detail above, non-circular stents may have primary tissue contact in the non-circular configuration thereof on major-axis end walls 395, wherein longer minor-axis sidewalls 396 may be configured to cyclically contact and pull, or be pulled, away from the adjacent blood vessel wall. Therefore, as it may be desirable to maintain side-branch-irrigating covering fenestrations and/or stent branches in relatively continuous contact and/or position relative to the adjacent blood vessel wall, it may be desirable to implement oval stents of the present disclosure in a manner such that side-branch fenestrations and/or auxiliary graphs/stents thereof are associated with major-axis ends of the oval form. Such positioning of covering fenestrations and/or branch stents at major-axis end walls of the stent can facilitate continuous contact/position of the fenestration(s)/branch(es) with the branch ostia and/or adjacent blood vessel wall/area.

In some anatomy, side branches of a primary blood vessel may project from opposite sides of the blood vessel, such as from the left and right sides of the aorta or other blood vessel. Therefore, by orienting an oval stent such that major-axis sides 395 thereof are positioned on left and right sides, respectively, of the aorta or other target blood vessel, side-branch fenestrations/stents on the major-axis sides 395 of the oval shape of the stent can align with the branch vessel opening(s) to promote continuous perfusion of such vessel side branches while allowing for cyclical deflection of the minor axis walls 396 without pulling the major-axis walls 395 away from the vessel walls and side-branch ostia. Implementation of covered oval stents having major-axis fenestrations and/or side-branch stent projections may be particularly suitable for abdominal implantation, where certain critical aortic branch vessels may emanate from the aorta on one or more sides thereof.

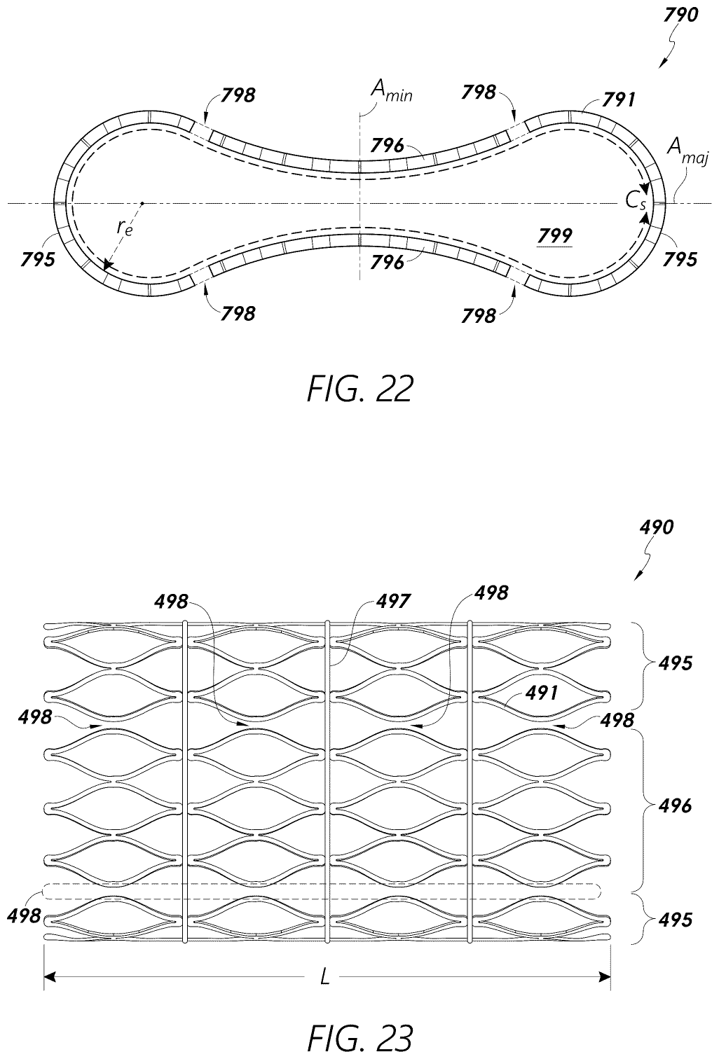

FIG. 22 shows an axial view of a non-circular stent 790 having circumferential hinge features 798 in accordance with one or more examples. The non-circular stent 790 may be configured such that the hinge features/lines 798 are distributed about a circumferential dimension Cs of the frame 790. The hinge features 798 may be positioned and/or disposed in areas separating/between adjacent minor-axis sidewalls 796 and major-axis end walls 795. That is, the circumference C, of the stent frame 790 can be divided into four quadrants or sections, each section separated from one or more adjacent circumferential sections by a hinge feature 798.

As described in detail above, examples of the present disclosure can include non-circular stents including certain coverings that distribute internal forces against the covering and/or stent frame, wherein such forces are exerted radially outwardly against the longer and/or straighter sidewalls 796 that are oriented generally parallel with the major-axis Aₘₐⱼ, as well as against the end walls 795, which may be curved and/or align generally more closely with the minor axis Aₘᵢₙ and connect between the sidewalls 796.

As the sidewalls 96 are outwardly deflected due to pressure forces in the lumen 799, the mechanical stress/force in the sidewalls 796 can be transferred/translated from the sidewalls 796 to the curved end walls 795. Such translation of mechanical reshaping force in the sidewalls 796 to the end walls 795 can cause the radius of curvature rₑ of the end walls 795 to increase, resulting in a wider shape of the frame 791 in the minor-axis dimension Aₘᵢₙ. In order to maximize volume change of the channel/lumen 799, it may be desirable to maintain a relatively narrow/thin profile of the frame 791 in the minor-axis dimension Aₘᵢₙ as the sidewalls 396 are outwardly deflected. The hinge features 798 may provide structural boundaries/breaks between the end walls 795 and the sidewalls 796, thereby reducing the translation of mechanical forces from the sidewalls 796 to the end walls 795.

Mechanical interruption introduced by the hinge features 798 can disrupt the otherwise continuous transition between such wall segments that can cause reshaping forces to impart undesirable widening of the curved end wall segments 795. That is, the stent 790 can be divided into quadrants that include hinge-lines formed at the boundaries between the side 796 and end 795 sections. These hinge features/lines 798 can allow the outwardly directed forces imparted by the elevated blood pressure in the lumen 799 to independently act against the different sections 796, 795 and avoid exertion of lever/moment between circumferential quadrants/sections.

The hinge features/lines 798 can be implemented in any number of ways, such as by designing the cells and/or struts of the stent frame 791 along the area/line 798 to act as weakened lines or links. For example, the hinge features/lines 798 may comprise gaps in the stent frame structure, or may be formed/configured by using thinner and/or weaker strut shape and/or thicknesses in such areas. In some implementations, a laser-cut pattern of the stent frame 791 may be designed to be more dense with respect to struts and cells of the stent frame in some areas and less dense and/or more flexible in others. For example, strut(s) within the sections 798 may be produced in a manner as to minimize the lever from the sidewalls 796 to the end walls 795, and vice versa, while still providing a continuous stent structure spanning across the circumferential sections 798. That is, the hinge features/spaces 798 may have relatively less metal/material passing therethrough than in other circumferential areas of the stent 790 to decrease force transfer across the sections 798.

For bare-frame stent implementations, may be desirable for forces of deflection of the end walls 795 to translate into outward deflection force in the sidewalls 796 to promote transition between oval and circular shapes. However, with respect to examples of oval stents comprising coverings that distribute the luminal pressure forces in a manner as to directly push/deflect outward the sidewalls 796, such translation of mechanical forces from the end walls 795 to the sidewalls 796 may not be necessary or desirable. Therefore, implementation of hinge features 798 may be particularly suitable for non-circular stent implant devices of the present disclosure that comprise covering features, as described in detail herein.

FIG. 23 shows a side view of a non-circular stent 490 having circumferential hinge features 498 in accordance with one or more examples. The stent 490 includes a frame 491 comprising a plurality of struts arranged to form cells therebetween as with other examples disclosed herein. The hinge features 498, which may be formed in circumferentially-offset positions about the circumference/perimeter of the frame 491 in a manner as to provide a mechanical disruption/discontinuity between adjacent circumferential segments 496, 495, can be formed of physical gaps, which may be implemented by adding or removing material from an otherwise continuous circumferential structure of the frame 491. The hinge features/gaps 498 may extend longitudinally across at least a portion of a length L of the stent 490, as shown. The gaps 498 may be present between circumferentially-adjacent cells 492 of the frame 491, such that the gaps separate columns of cells of the frame.

In some examples, the adjacent circumferential segments 496, 495 of the frame 491 may be secured to one another via circumferentially-oriented connecting struts 497, which may span circumferentially across the gap areas 498 of the hinge line/features, as shown. The connectors 497 may comprise relatively thin splines or struts that run through the gaps 498 and connected between struts of adjacent circumferential sections 496, 495 of the frame 491.

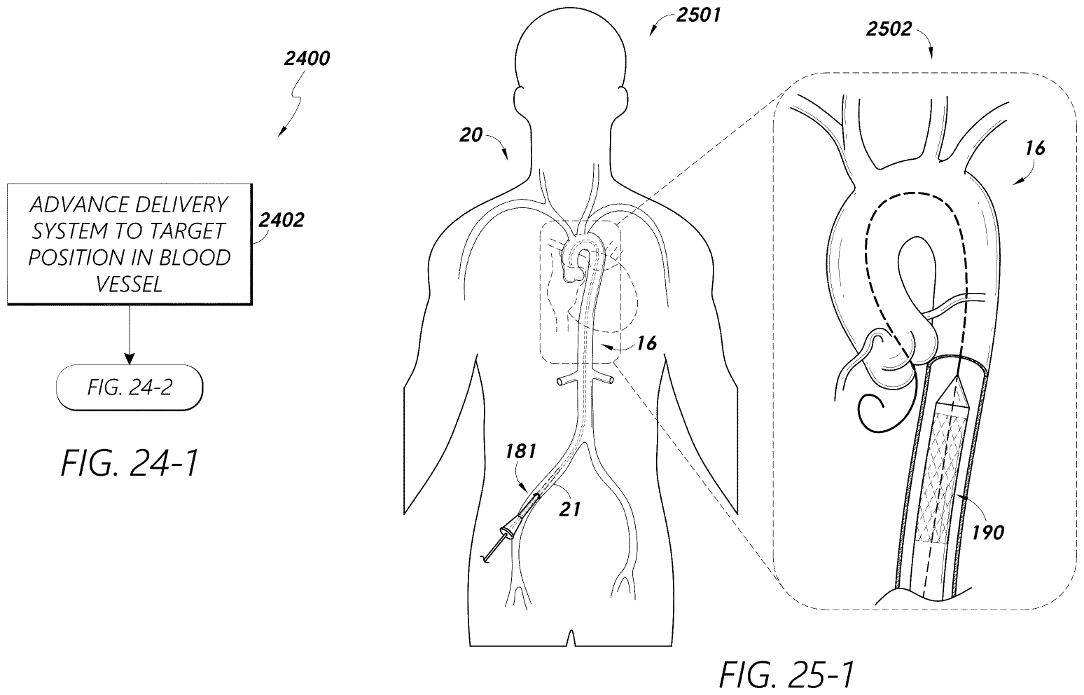

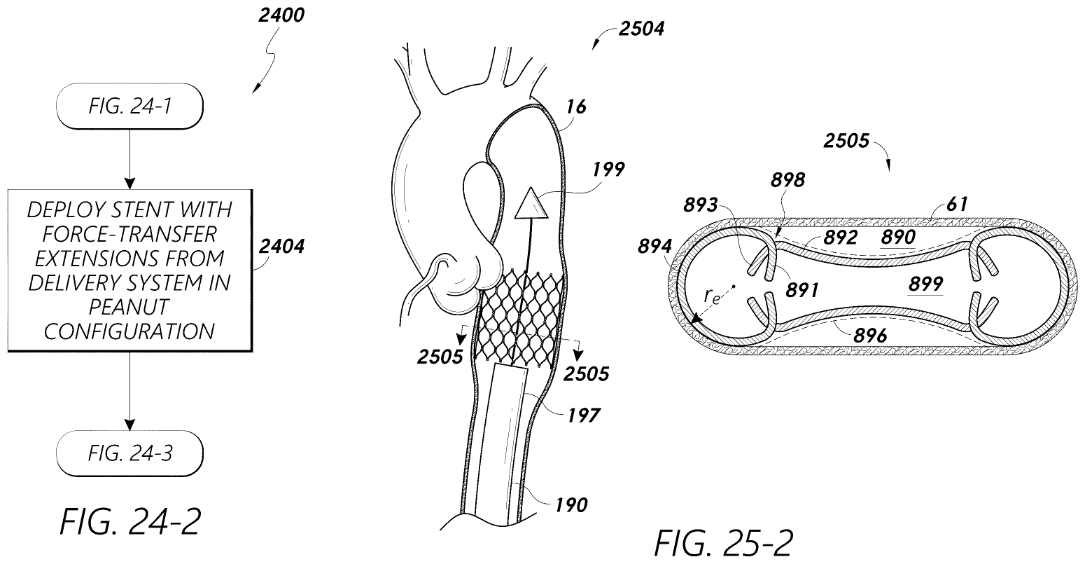

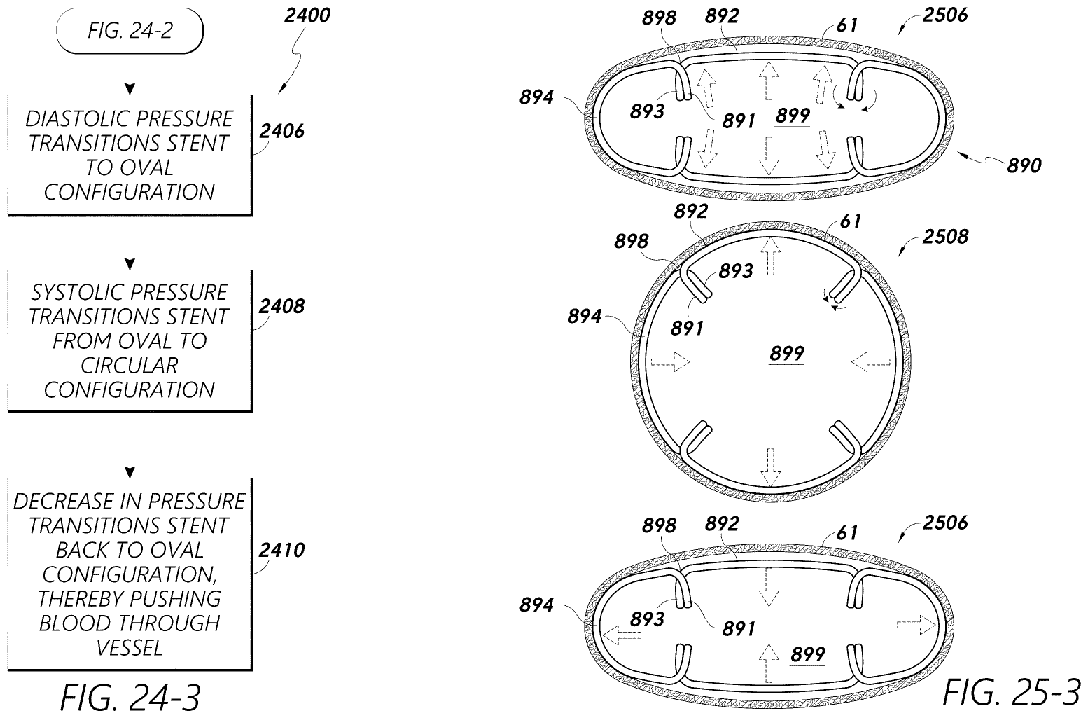

FIGS. 24-1, 24-2, and 24-3 illustrate a flow diagram for a process 2400 for reshaping a blood vessel 16 using a non-circular stent 890 including hinge 898 and stopper 893 features in accordance with one or more examples. FIGS. 25-1, 25-2, and 25-3 provide images of the stent and certain anatomy corresponding to operations of the process 2400 of FIGS. 24-1, 24-2, and 24-3 according to one or more examples.

At block 2402, the process 2400 involves advancing a delivery system 190 to a target position in a blood vessel 16, such as the aorta. For example, the delivery system 190 may be advanced through a percutaneous introducer or other minimally-invasive access 181 into the vasculature of the patient, and further within the vasculature to a target position within the aorta 16 of the patient. The delivery system 190 may include one or more catheters/sheaths 197 and/or a nosecone 199 or other feature configured to facilitate the forward advancement of the delivery system 190 through tortuous anatomy of the vasculature. The percutaneous entry 181 may be at the femoral artery or other arterial blood vessel.

At block 2404, the process 2400 involves deploying an oval (e.g., peanut-shaped) stent 890 from the delivery system 190, wherein the stent 890 is configured to assume a peanut-shaped configuration in a relaxed, non-pressurized state thereof. The stent 890 may be transported to the implantation site in a radially crimped/compressed configuration, wherein the stent may expand in accordance with self-expansion when deployed from the delivery system 190, or may be expanded using a balloon catheter or similar device. The image 2504 shows the stent 890 deployed in the blood vessel 16, whereas the image 2505 provides an axial cross-sectional detail of the stent 890. The stent 890 is a covered stent having a covering 896 on an inner or outer surface thereof, such that luminal pressures in the channel 899 cause outward deflection of the sidewalls 892 without requiring force transfer from the end walls 894 to the sidewalls 892 across the hinge lines 898.

As shown in the image 2505, the stent 890 may include hinge features 898, which may have any of the features of hinge features disclosed herein, wherein the hinge features 898 may provide a circumferential structural discontinuity between circumferentially-adjacent minor-axis sidewalls 892 and major-axis end walls 894 of the stent frame. The hinge features 898 may be desirable to eliminate the transfer of lever forces between circumferentially-adjacent stent frame segments, though the elimination of such force transfer may be desirable only to a certain point and/or with respect to certain shapes or configurations of the stent 890. For example, when expanding to a circular shape, it may be desirable to transfer forces from sidewalls 892 to end walls 894 to increase a radius of curvature rₑ thereof.

As described above, circumferential hinge features can be desirable to prevent outward deflection forces on the sidewalls 899 from causing expansion of the radius of curvature r, of the end walls 894. Such expansion risk may primarily be a concern when the stent 890 is in an oval configuration during diastole. However, as described a block 2406, with respect to covered peanut-shaped stents, minimum diastolic pressures in the target blood vessel may cause outward deflection of the sidewalls 892 to shape the sidewalls 892 into a flatter configuration. During expansion of the sidewalls 892 from the inwardly-deflected concave configuration of image 2505 to the flatter configuration of image 2506, it may be desirable to prevent such deflection of the sidewalls 892 from transferring force across the hinge line 898 in a manner as to enlarge the radius rₑ of the curved and walls 894. Therefore, the hinge features 898 can be desirable as preventing such effect on the end walls 894 when the sidewalls 892 deflect outwardly to the flatter/oval configuration during diastole.

After transition to the oval configuration of image 2506, it may be desirable to allow/promote mechanical force transfer between the sidewalls 892 and the end walls 894 to assist in reshaping of the end walls 894 to more-circular curvature as the sidewalls 892 are deflected to more-circular curvature. Therefore, the stent 890 may advantageously include certain extension features 891, 893, which may serve to increase the force transfer between the sidewalls 892 and the end walls 894 when the sidewalls 892 have been outwardly deflected to some degree as in image 2506. For example, the extensions 891. 893 may serve as interference structures that move with respective associated wall portions. For example, the stopper extension 891 may be associated with the end wall 894, such that inward and/or outward deflection of the end wall 894 causes the extension stopper 891 to deflect in a commensurate manner. Furthermore the extension stopper 893 may be mechanically coupled and/or otherwise associated with the side wall 892, such that outward deflection of the side wall 892 causes deflection (e.g., counterclockwise deflection with respect to the illustrated orientation), wherein when the sidewalls 892 are outwardly deflected to the flatter configuration of the diastolic pressure state of the stent 890, the extension stoppers 891, 893 are deflected towards one another and into physical contact with one another, such that further deflection of the end walls side wall 892 causes exertion of force across the hinge line 898, such that outward deflection of the sidewalls 892 causes inward deflection of the end walls 894 to facilitate the transition of the shape of the stent 890 from the oval shaped shown in image 2506 to a more circular shape shown in image 2508.

The extension stoppers 891, 893 may comprise internally-directed, angled extensions, which separately extend from the ends of the circumferential wall segments 892, 893, respectively. The angles of the extensions 891, 893 may be designed to allow the concave sides 892 to straighten under diastolic pressure without exerting lever forces across the hinge lines 898 to the arched and walls 894 on the major axis, thereby keeping the arched end walls 894 at a desirable/minimum radius rₑ during diastolic deflection of the sidewalls 892. After deflection of the sidewalls 892 to the flatter configuration shown in image 2506, the extensions 891. 893 may lock/contact against one another, thereby bringing the circumferential sections of the stent frame into interaction with one another to facilitate transition of lever forces across the hinge lines 898 to allow for expansion to a circular cross-section in the high-pressure phase of the cardiac cycle.

By implementing the extension stoppers 891, 893, the outward deflection of the sidewalls 892 during diastole may not result in the enlarged expansion of the radius rₑ of the curved end walls 894, whereas in transitioning from diastole to systole, due to the interference of the extension stoppers 891, 893 with one another, the outward deflection of the sidewalls 892 from the flatter configuration out towards a more circular convex configuration can allow for transfer of force to the sidewalls 894 in a manner as to inwardly deflected such walls and facilitates a more circular shape of the end walls 894 in combination with the sidewalls 892, as shown in image 2508. With the stopper extensions 891, 893 in contact with one another, the entire circumference of the stent frame can act as a continuous body irrespective of the hinge lines/features 898, such that the deflection of the various wall segments exerts normal forces/levers across the hinges 898. The interference contacts/extensions 891, 893 optimize efficacy in terms of volume change of the stent 890 by promoting greater reshaping of the stent frame and cooperation between the circumferential stent frame segments during systole.

During diastole, the diastolic pressure baseline, which represents a minimum pressure of the cardiac cycle, deflect/straightens the waste of the stent 890 formed by the inward/concave deflection of the sidewalls 892, wherein such straightening/deflection brings the stoppers 891, 893 into the interference contact shown in image 2506. Interference contact of the stoppers 891, 893 may further prevent buckling of the sidewalls 892 in some instances where the sidewalls 892 are prone to such buckling. For example, such buckling may be prevented by contact between the side wall 892 and the stopper 891, which may physically prevent further inward deflection of the side wall 892.

At block 2408, the process 2400 involves transitioning of the stent 890 from the oval state to the circular state in response to increase systolic pressures, wherein the contact between end wall and side wall extension stoppers facilitates the transfer of expansion forces between circumferentially-adjacent segments/portions of the stent frame to promote circular expansion.

The stopper extensions 891, 893 allow for the outward deflection of the sidewalls 892 without substantially enlarging the minor diameter of the stent, thereby allowing for the stent to keep a relatively thin oval shape in the diastolic state shown in image 2506. For diastolic pressure, it may be desirable to prevent excessive widening of the oval shape of the stent, to thereby keep the profile/minor axis of the oval as low as possible while allowing for the concave sidewalls 892 to deflect to a flatter configuration. Therefore, neutralization of the deflection of the sidewalls 892 from the physical transfer of such forces to cause deflection of the end walls 894 can be desirable. The stent 890 is configured such that the deflection of the concave inwardly-deflected walls 892 outward straightens the sidewalls 892 without enlarging the radii of the ends of the stent, thereby producing a relatively thin oval shaped in the diastolic state. Once the thin oval shape shown in image 2506 is achieved in connection with the low-pressure phase of the cardiac cycle, it may be desirable to maximize the transition from the thin oval shaped to a full, round/circular shape shown in image 2508, and therefore the greater transfer of forces circumferentially through the stent frame between the diastolic and systolic phases may be desirable, such differential force transfer throughout the cardiac cycle being enabled by the configuration of the stent 890.

At block 2410, the process 2400 involves the decrease in pressure from the systolic phase back to the diastolic phase causes the stent 890 to transition back to a more-oval configuration, which can serve to push blood through the blood vessel and increase diastolic pressure and flow.

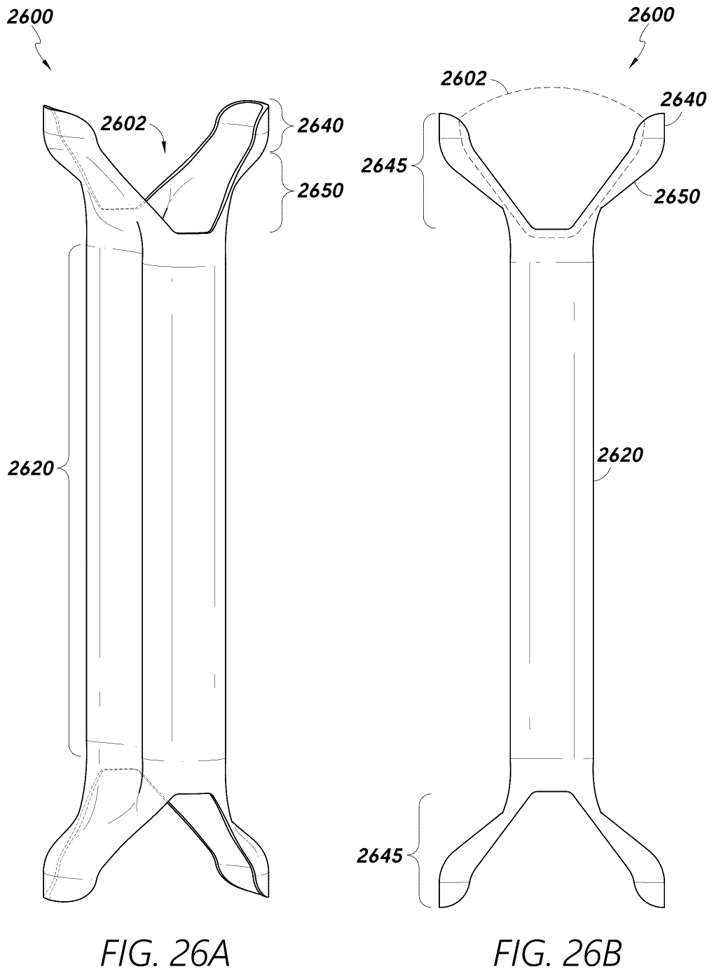

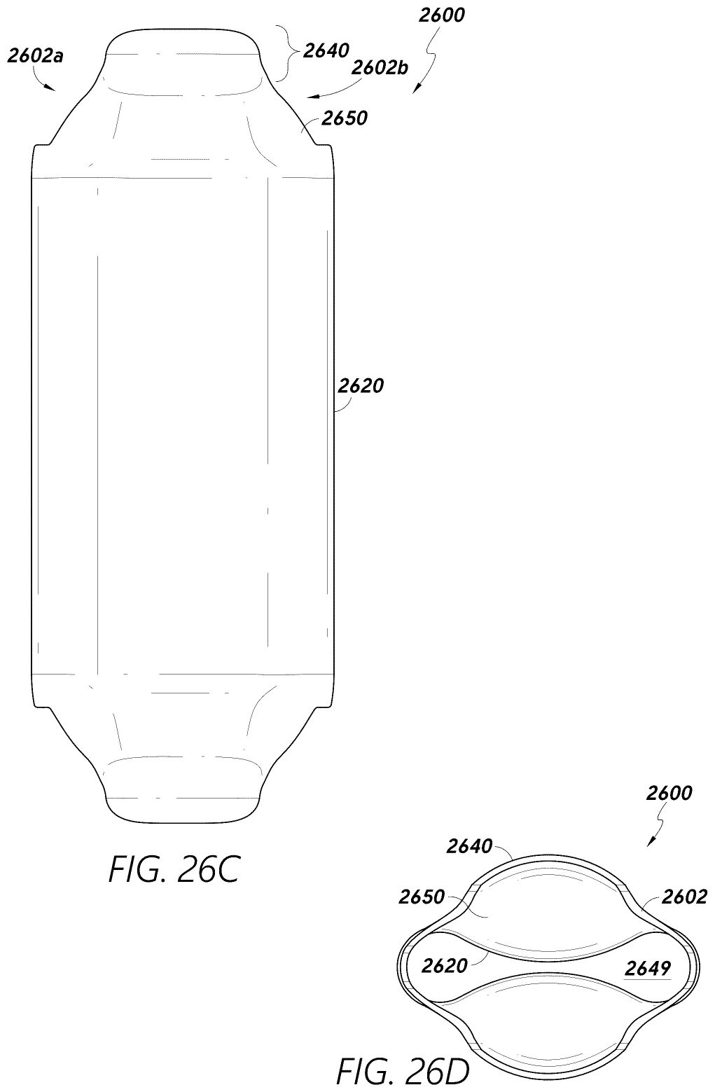

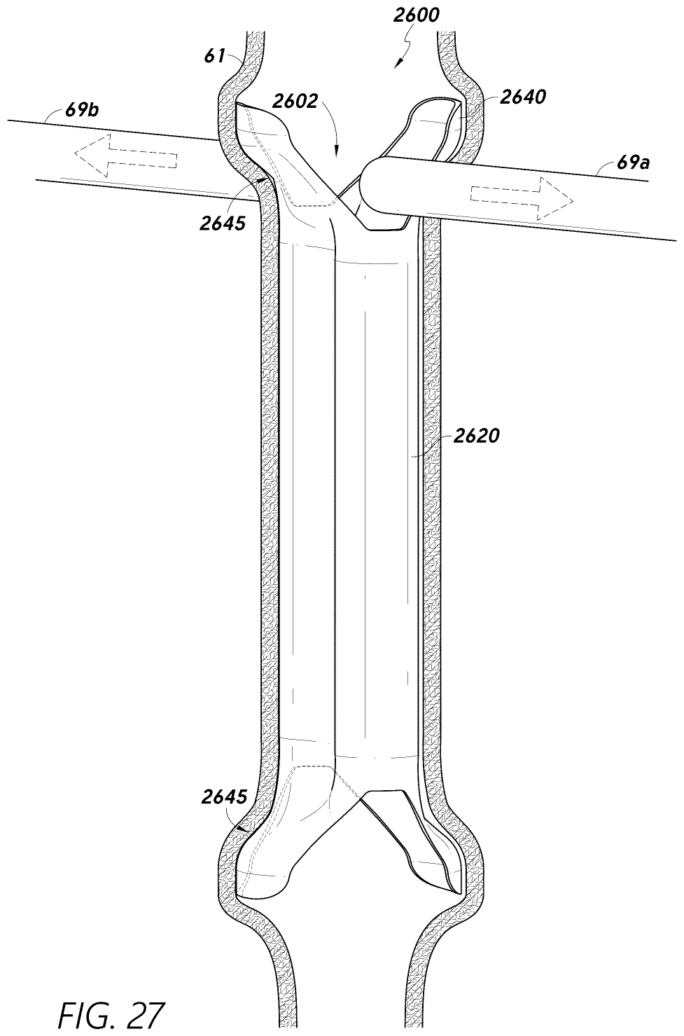

FIGS. 26A-26D show perspective, side, and axial views, respectively, of a stent 2600 having circular end portions 2645 and a non-circular medial portion 2620, wherein the end portions 2645 include scallop features in accordance with one or more examples. The stent 2600 may comprise stent frame and/or covering components, as with any of the other examples disclosed herein, though such components are not illustrated particularly in FIGS. 26A-26D and FIG. 27 for the purpose of visual clarity. That is, although the stent 2600 is illustrated as a solid, planer form in the form/shape of a tubular stent having various circular and non-circular cross-sectional shapes, it should be understood that such illustrated form may be formed by a stent frame having struts and open cells as described in detail herein, wherein such frame may or may not be covered by a fluid-tight covering on an outer and/or inner aspects thereof.

The stent 2600 may be similar to certain other stents disclosed herein, in that a stent 2600 includes axial end portions 2640 that may have a generally-circular cross-sectional curvature, which may serve to provide hemostasis-promoting contact with the target blood vessel in which the stent 2600 is implanted. Furthermore, the stent 2600, as with other examples disclosed herein, may include a non-circular medial portion 2620, which may have an oval and/or peanut-shaped cross-sectional form, as described in detail herein. The stent 2600 may further comprise transition portions 2650 where the shape/form of the stent 2600 transitions from the circular end shape 2640 to the non-circular medial portion shape 2620. The axial ends 2640 and transition portions 2650 may together be referred to herein as the ends (or end portions) 2645 of the stent 2600.

The stent 2600 may further include certain scallop features 2602, which may comprise cut-out portions of the structure of the circular end portions 2640 and/or transition portions 2650 in/on one or more sides/areas of the stent. For example, as illustrated, the ends 2645 of the stent 2600 may comprise two, mirrored scallop features 2602 on opposite circumferential sides of the stent 2600. The scallop feature(s) 2602 may be aligned with and/or occupy the major-axis sides of the stent 2600, with respect to the major-axis sides/ends of the non-circular medial portion 2620. The scallop features 2602 may have any desirable shape or configuration. Furthermore, the scallop features 2602 may be cut-out from any area of the end portions 2645, or from the medial portion 2620 of the stent 2600.

FIG. 27 shows the stent 2600 with scalloped end portions 2645 implanted in a target blood vessel 61 in accordance with one or more examples. The scallop features 2602 may accommodate the presence of side branch(es) 69 emanating from the blood vessel 61. For example, the stent 2600 may be implanted in an area of the blood vessel 61, such that at least one of the end portions 2645 of the stent 2600 axially overlaps one or more side branches 69, wherein the scallop feature(s) 2602 of the stent 2600 are positioned such that the scallop(s) 2602 provide fluid access to the side branch(es) 69. That is, the implementation of scallop feature(s) 2602 prevent the end portion(s) 2645 from blocking/obstructing fluid flow from within the primary lumen of the blood vessel 61 from passing into the side branch(s) 69. Therefore, with the stent 2600 implemented with scallop features 2602, as shown, the stent 2600 may advantageously provide a hemostasis seal on axial ends of the medial portion 2620 in the area of the side branch(s) 69, while maintaining open fluid passage into the side branch(s). Any of the stent devices disclosed herein may be implemented with one or more scallop features.

### Stent- and Vessel-Shaping Balloon Devices

Various solutions are presented herein of stents and other vessel-reshaping devices that can increase compliance in blood vessels of, for example, hypertensive patients (including heart failure patients). For example, disclosed above are non-circular stent solutions for adding compliance to the arterial system. Such stent devices can be configured to be biased toward an oval (or other non-circular) shape under relatively low-pressure conditions. Under higher (e.g., systolic) pressure conditions, the aorta or other blood vessel in which the stent is implanted can be deformed into a more circular shape, after which the stent is configured to revert the blood vessel back toward the non-circular (e.g., oval) shape. In this manner, the non-circular stent can be configured to restore some compliance to an otherwise insufficiently-compliant blood vessel (e.g., aortic) segment.

Some stent solutions are provided/configured as a ‘one-size-fits-all’ solution, in that the stent is configured with a specific geometry (e.g., size) and/or rigidity. Such solutions may be less suitable or effective for certain anatomies that present unique geometries, for which customized, case-specific stent expansion geometry and/or rigidity may be advantageous. In some implementations, vessel-reshaping solutions of the present disclosure provide dynamically-controllable shape and/or rigidity in a manner as to better conform to specific anatomical structures.

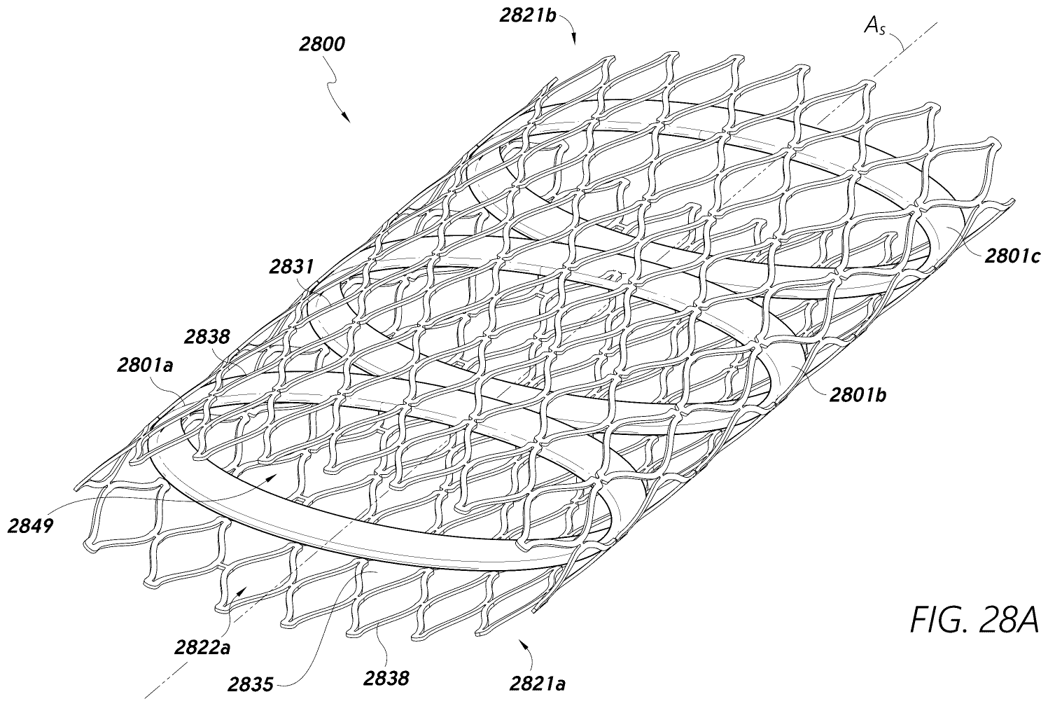

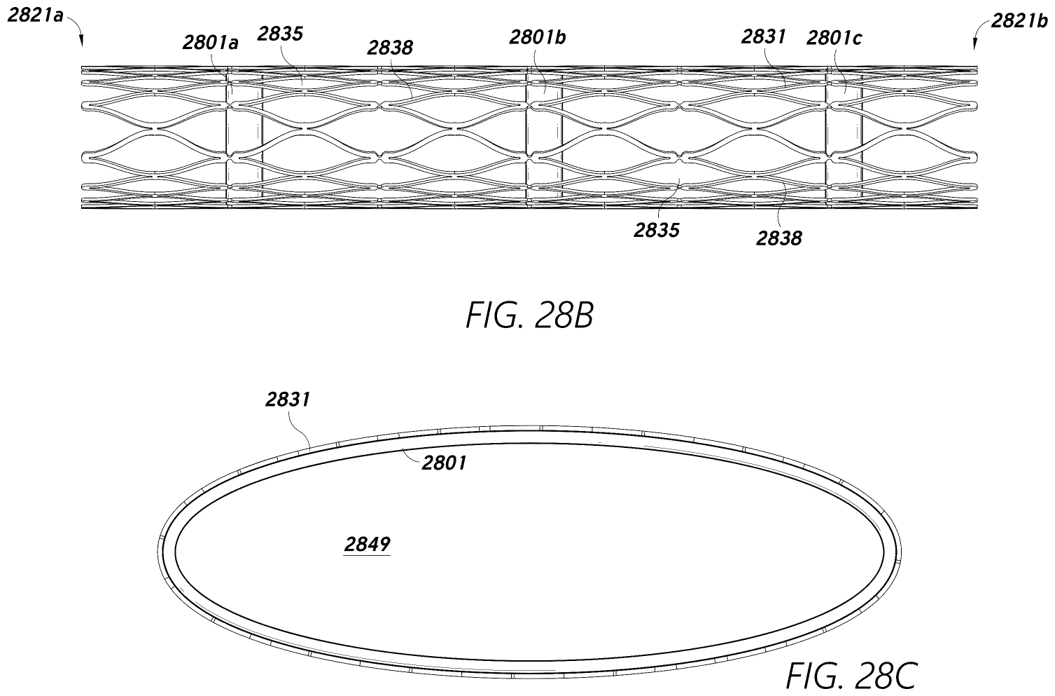

FIGS. 28A-28C show perspective, side, and axial views, respectively, of a stent 2800 having one or more balloons associated therewith in accordance with one or more examples. The stent 2800 may have the features of any of the example non-circular stents disclosed herein. For example, the stent 2800 may comprise a stent frame 2831 formed of an arrangement of struts 2838 arranged to form a plurality of rows and/or columns of cells 2835. The stent 2800 may or may not have a covering as disclosed herein.

The stent 2800 includes one or more donut-shaped balloons disposed within or without the frame 2831. The balloon(s) 2801 can be physically attached/secured to the frame 2810, or may be separate components that are simply disposed/positioned within or without the frame 2810. The balloon(s) 2801 can be inflatable, such that they may be inflated within (or without) the stent 2800 to effect expansion and/or shaping of the frame 2810 to conform to the shape of the inflated/expanded balloon(s) 2801. Although three axially-offset balloons 2810 are shown, including a first balloon 2801a in an area of a first axial end 2821a of the stent 2800, a second balloon 2801b disposed in a medial area/portion of the stent 2800, and a third balloon 2801c disposed in an area of a second axial end 2821b of the stent 2800, it should be understood that stent re-shaping balloons may be positioned or associated with any axial position of a stent, and may further be arranged in any number of balloons and in any position or combination of positions. The balloon(s) 2801 may be inflated such that a degree of inflation of the individual balloon(s) dictates, at least in part, a deployed shape and/or rigidity of the stent 2800.

The balloon(s) 2801 can be hollow, donut-shaped balloons, which can be inflated inside the stent after deployment of the stent in the target blood vessel. The degree to which the balloon(s) 2801 is/are inflated can control the shape and/or rigidity of the stent, thereby providing the surgeon/technician to control such parameters based on the specific anatomical characteristics at the site of implantation. The donut-shape of the balloon(s) 2801 can permit blood-flow therethrough with relatively low flow disturbance, as blood may flow through a central flow channel 2849 of the balloon(s) 2801.

The implementation of multiple axially-offset balloons, as shown in FIG. 28A, can advantageously provide control not only over the shape and rigidity of the stent 2800 as a whole, but can allow for shaping of the stent 2800 in different shapes and/or rigidities along different axial portions/lengths of the stent 2800 to better conform to the target anatomical structure, which may vary in size, shape, and/or dimension over the area/length spanned by the implanted stent.

The use of the expansion balloon(s) 2801 can promote conformal contact between the frame 2831 and the blood vessel wall, thereby facilitating tissue ingrowth and/or other attachment or secure frictional interference between the frame 2831 and the blood vessel wall to secure the stent 2800 in-place in the target position in the blood vessel. As with any of the stent- and/or vessel-shaping balloons disclosed herein, the balloon(s) 2801. individually or collectively, can span substantially the entire circumference and/or length of the stent frame 2831, or only a portion of the circumference and/or length. The balloon(s) 2801, as with any other balloon devices disclosed herein, may comprise any suitable or desirable material. For example, the balloon(s) 2840 may comprise a compliant or non-compliant material (e.g., polymer).

With respect to any vessel- and/or stent-reshaping balloons disclosed herein, such balloons can advantageously comprise or be associated with one or more detachable inflation one-way nozzles that is/are fluidly coupled with/to an external delivery device that may be utilized for deployment purposes. Furthermore, any balloon device disclosed herein may comprise a plurality of segments arranged in parallel to address necessary or desired variations in shape, size, and/or configuration along the vessel length.

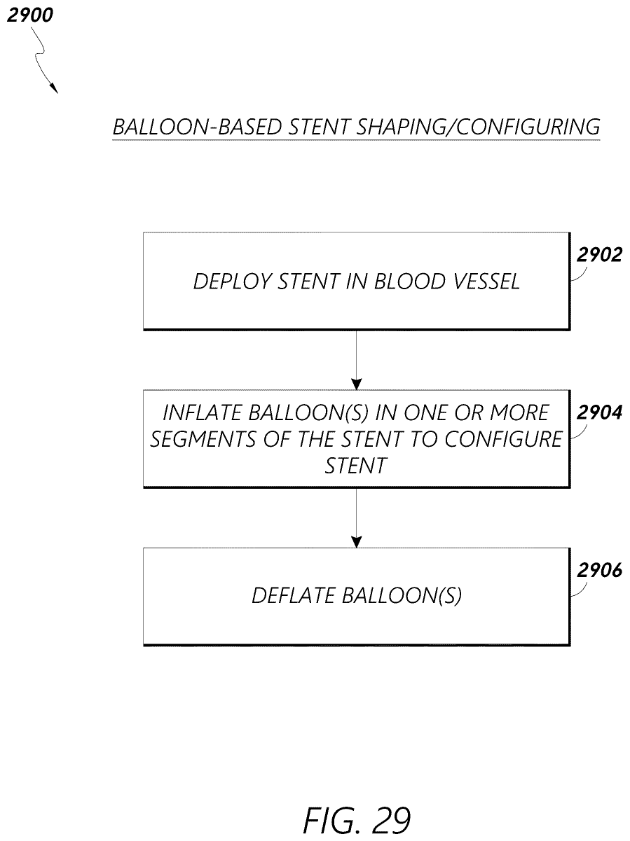

FIG. 29 is a flow diagram illustrating a process 2900 for shaping a stent using one or more balloon devices in accordance with one or more examples of the present disclosure. The process 2900 may be implemented using any of the stent and/or balloon devices disclosed herein.

At block 2902, the process 2900 involves deploying a stent in a target blood vessel, such as a segment of the aorta or other arterial blood vessel. The stent may be, for example, a stent having a biased non-circular shape, which may allow for compliance-enhancing blood vessel reshaping using the stent as described herein. Alternatively, the stent may have a biased circular shape, wherein the stent may be configured to be reshaped by one or more balloon devices to a non-circular cross-sectional shape after deployment in the target blood vessel.

At block 2904, the process 2900 involves inflating one or more balloons within and/or without one or more segments of the stent to thereby shape and/or otherwise configure the stent with respect to rigidity or other feature(s) thereof. The reshaping of the stent in accordance with block 2904 may be implemented using a plurality of axially-offset balloon devices, or a single tubular balloon device, as disclosed in detail herein. The balloon(s) used to expand the stent may be tubular in shape in that they form an axial channel through which blood can flow during shaping of the stent.

The balloon(s) used to shape and/or otherwise configure the stent can be pre-attached to the stent prior to deployment of the stent in the blood vessel, or may be introduced into the lumen of the stent after deployment thereof. Furthermore, the balloon(s) may be detachable from the stent to allow for removal of the balloon(s) after shaping of the stent.

Different balloons disposed within and/or outside of the stent channel may be inflated to different volumes and/or shapes to create non-uniform shapes, rigidity, and/or other characteristics of the implanted stent along the length of the stent. For example, in some implementations, balloons associated with end portions of the stent may be inflated to a circular shape to create circular and shapes for the stent, whereas one or more balloons in a medial portion of the stent may be inflated to a non-circular stent, such as an oval-shaped or other shape. In such cases, the circular shape of the ends of the stent may serve to create a desirable seal between the stent and the blood vessel, whereas the non-circular portion(s) may be configured to cyclically reshape the blood vessel to add compliance thereto.

At block 2906, the process 2900 involves deflating the one or more balloon(s) after the desired shaping/configuration of the stent has been implemented. Deflating of the balloon(s) may be implemented to allow for removal of the balloon(s) from the blood vessel. In some implementations, the balloon(s) may be maintained within or without the stent after shaping of the stent for an indefinite period after the implantation procedure. In such cases, deflation of the balloon(s) may reduce the volume thereof, thereby reducing the impact of the balloon(s) on blood flow within the vessel. The balloon(s) may be attached to the stent in some implementations, such as through suturing, adhesive, or other attachment means or mechanism. With the stent customizability shaped and/or configured in connection with the process 2900, the stent may be maintained in the blood vessel and serve to improve the compliance and/or blood flow characteristics associated therewith.

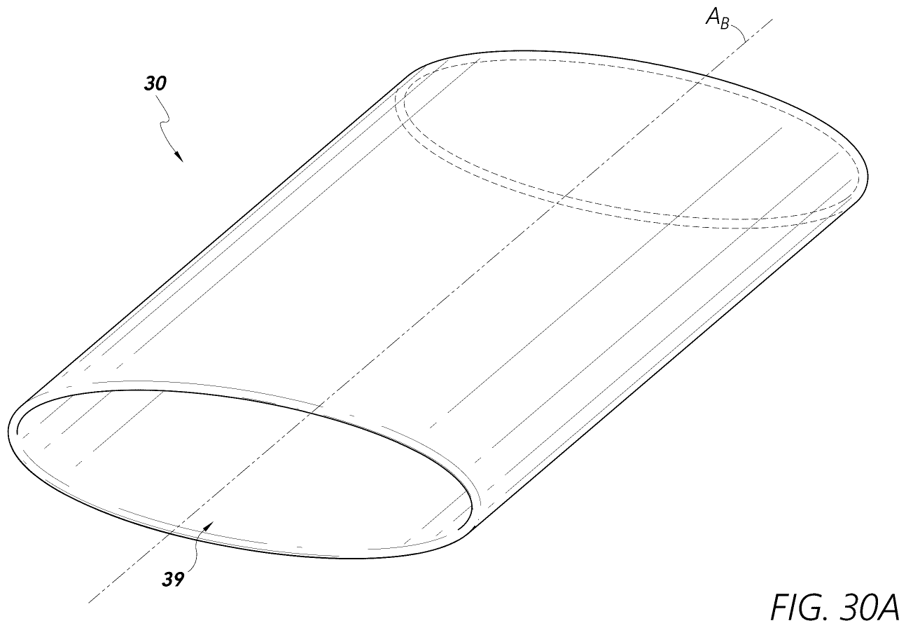

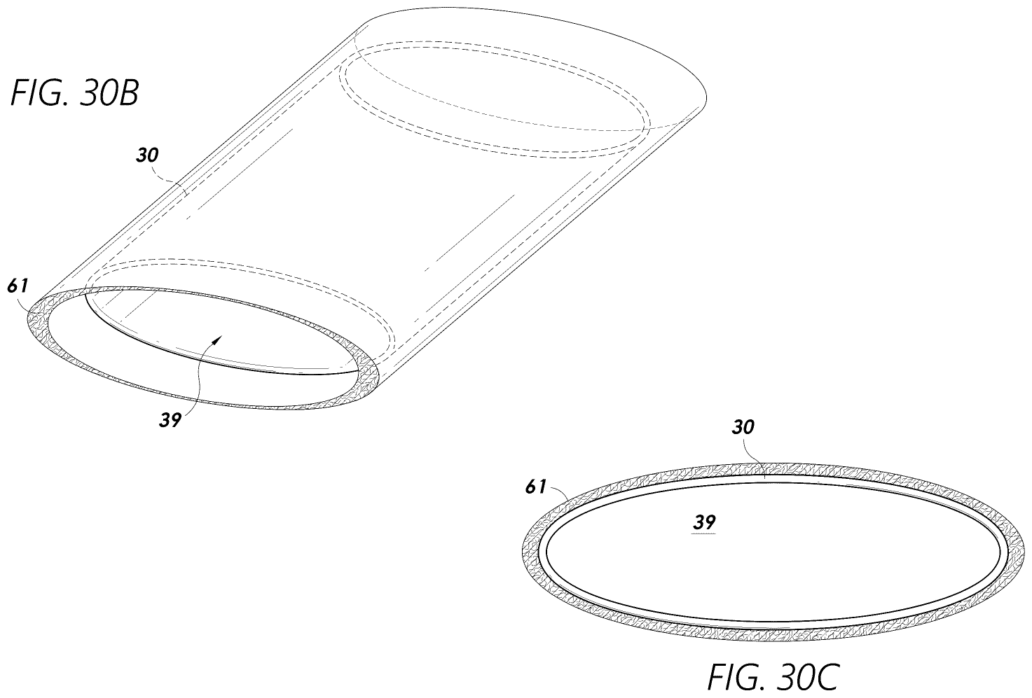

FIGS. 30A-30C show perspective, side, and axial views, respectively, of a vessel-reshaping balloon device 30 in accordance with one or more examples. The balloon device 30 may be tubular in shape, comprising wall(s) forming a central flow channel 39 about an axis A_{B} of the device 30. The balloon device 30, in an inflated state thereof as illustrated in FIG. 30A, may have a non-circular cross-sectional shape, such as an oval-shaped or other non-circular shape.

The balloon device 30 may be implanted as a permanent implant within a blood vessel, as shown in FIG. 30B, wherein the balloon may be at least partially inflated before and/or after deployment from a delivery system used to transport the device 30 to the target implantation site. For example, delivery processes associated with balloon devices as in FIGS. 30A-30C may involve deploying a balloon tube from the delivery system in a target segment of the blood vessel and subsequently inflating the balloon to a desirable inflation volume and/or shape, after which the balloon may be fluidly sealed such that the device 30 retains, in a permanent or semi-permanent manner, the shape and/or configuration thereof as inflated. When implanted, the implant device 30 may reshape the blood vessel 61 to the non-circular shape as shown in FIG. 30B and FIG. 30C during low-pressure conditions (e.g., diastole), wherein higher pressure within the channel 39 of the balloon device may cause circularization thereof, thereby reshaping the blood vessel to transition between less-circular and more-circular shapes to improve compliance as described in detail herein. That is, the balloon device 30 may function similar to the non-circular stents disclosed in detail herein, but may be implanted and perform such functionality without the need of a rigid mental stent frame. The circularization of the balloon during systole may be caused at least in part by the fluid pressure within the channel 39 of the device pushing outwardly against the minor-axis walls of the device 30, and/or the circularization/reshaping in systole may be caused by the re-shaping of the blood vessel in the areas axially outside of the balloon 30 caused by high blood pressure, such reshaping of the blood vessel causing the blood vessel to push inwardly on major-axis ends of the balloon 30 to cause reshaping thereof.

The implant device 30 may provide for control over the reshaping effect thereof by controlling the inflation volume of the balloon 30, such as during implantation. Furthermore, relative to metal-frame stents, the balloon device 30 may provide improved distribution of pressure forces exerted by the device on the surrounding blood vessel. The balloon device 30 additionally may be able and/or configured to crimp to relatively smaller sizes and/or provide improved flexibility compared to certain metal-frame stents. However, the balloon device 30 may present certain risks associated with rupture thereof. Therefore, it may be desirable to fill the balloon with biocompatible gas/fluid, such as CO2, sailing fluid, or the like. In cases where the balloon 30 undesirably ruptures, such event may effectively defeat the compliance-enhancing functionality of the device.

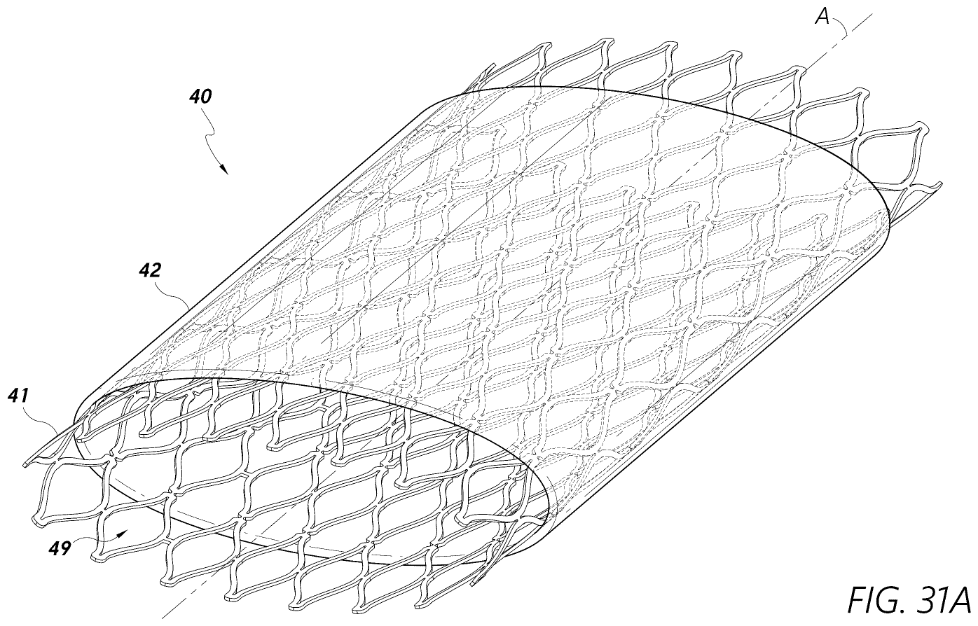

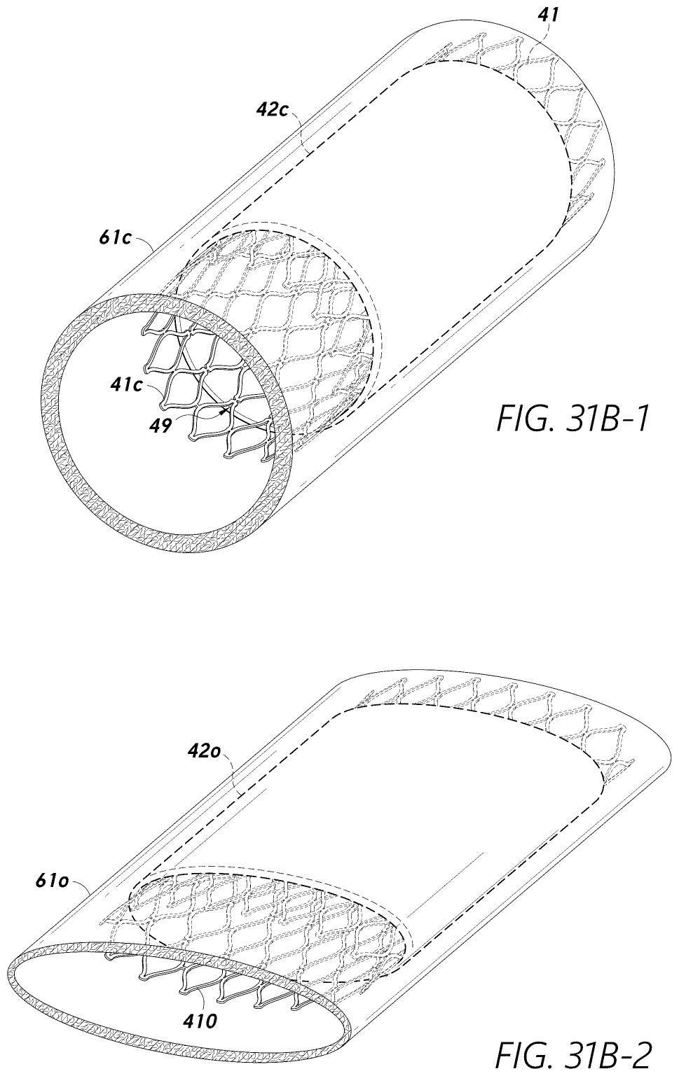

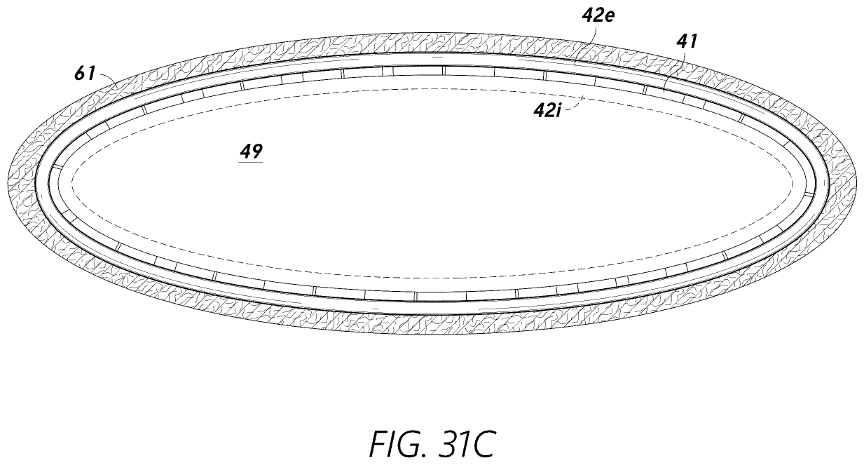

FIGS. 31A and 31C show perspective and axial views, respectively, of a stent-reshaping balloon 42 reshaping a stent in accordance with one or more examples. FIGS. 31B-1 and 31B-2 show the stent-reshaping balloon 42 of FIGS. 31A and 31C in a blood vessel 61 in circular and non-circular configurations, respectively, in accordance with one or more examples. The balloon 42 may span at least 50% of the length of the sent frame 41 in some implementations.

The balloon device 42, which may be associated with a stent 41 in a combined assembly 40, may be tubular in shape, comprising wall(s) forming a central flow channel 49 about an axis A_{B} of the device 40. The balloon device 42, in an inflated state thereof as illustrated in FIG. 31A, may have a non-circular cross-sectional shape, such as an oval-shaped or other non-circular shape. In some implementations, the balloon 42 has an extremely-ovalized shape with a major axis that is at least twice as long as a minor axis thereof. The balloon 42 may be either external to the stent 41, as shown, or may be internal to the stent 41. In some implementations, balloon devices may be disposed within and without the stent 41, wherein such balloon devices may be separate or integrated balloons/portions.

As shown in FIG. 31B-1, the stent 41c and balloon 42c may be implanted in a blood vessel initially in a circular shape conforming generally to the shape of the circular blood vessel 61. For example, the stent 41c may be deployed in a circular shape within the blood vessel 61c, such that the blood vessel 61c is likewise generally circular with the stent 41c implanted therein. The balloon 42c may be implanted together with the stent 41c, such that the balloon 42c is disposed outside of the stent 41c in a deflated state prior to inflation of the balloon. Alternatively, the tube 42c may be introduced into an inner lumen of the stent 41c after deployment of the stent 41c.

As initially implanted/deployed, the stent 41c may have a relaxed circular shape, as shown in FIG. 31B-1. The balloon 42 may be inflated in a manner as to cause the balloon 42 to form a non-circular shape that is associated with a natural shape thereof as inflated. For example, as shown in FIG. 31B-2, the balloon 42o may ovalize as pressure increases therein from inflation, wherein such ovalization of the balloon 42 causes a commensurate reshaping/ovalization of the stent 41o. Therefore, the balloon 42 can advantageously reshape the stent 41 to the ovalized shape 41o thereof, wherein, once reshaped as shown in FIG. 31B-2, the stent 41o may serve as a non-circular, compliance-enhancing stent as described in detail herein. Furthermore, the balloon 42 may be maintained together with the stent 41, wherein the combination of the stent 41 and balloon 42 can provide desirable reshaping of the blood vessel 61o. As shown in the axial view of FIG. 31C, it should be understood that the balloon 42 may be position/disposed external to the stent 41, as shown in the illustrated balloon 42e. or alternatively or additionally, a balloon 42i may be disposed within the stent 41, wherein the balloon 41i can reshape the stent 41 in the manner described above.

The balloon 42 may be at least partially inflated before and/or after deployment from a delivery system used to transport the device/assembly 40 to the target implantation site. For example, the balloon 42 may be deployed from the delivery system, together with or subsequent to deployment of the stent 41, in a target segment of the blood vessel and subsequently inflated to a desirable inflation volume and/or shape to thereby reshape the stent 41, after which the balloon 42 may be fluidly sealed such that the device/assembly 40 retains, in a permanent or semi-permanent manner, the shape and/or configuration thereof as inflated. When implanted, the implant device/assembly 40 may reshape the blood vessel 61 to the non-circular shape as shown in FIG. 31B-2 and FIG. 31C during low-pressure conditions (e.g., diastole), wherein higher pressure within the channel 49 of the balloon device 42 and/or stent 41 may cause circularization thereof, thereby reshaping the blood vessel to transition between less-circular and more-circular shapes to improve compliance as described in detail herein. That is, the stent 41, as reshaped by the balloon 42, may function similar to other non-circular stents disclosed in detail herein. The balloon device 42 may provide for control over the reshaping effect thereof by controlling the inflation volume of the balloon 42, such as during implantation.

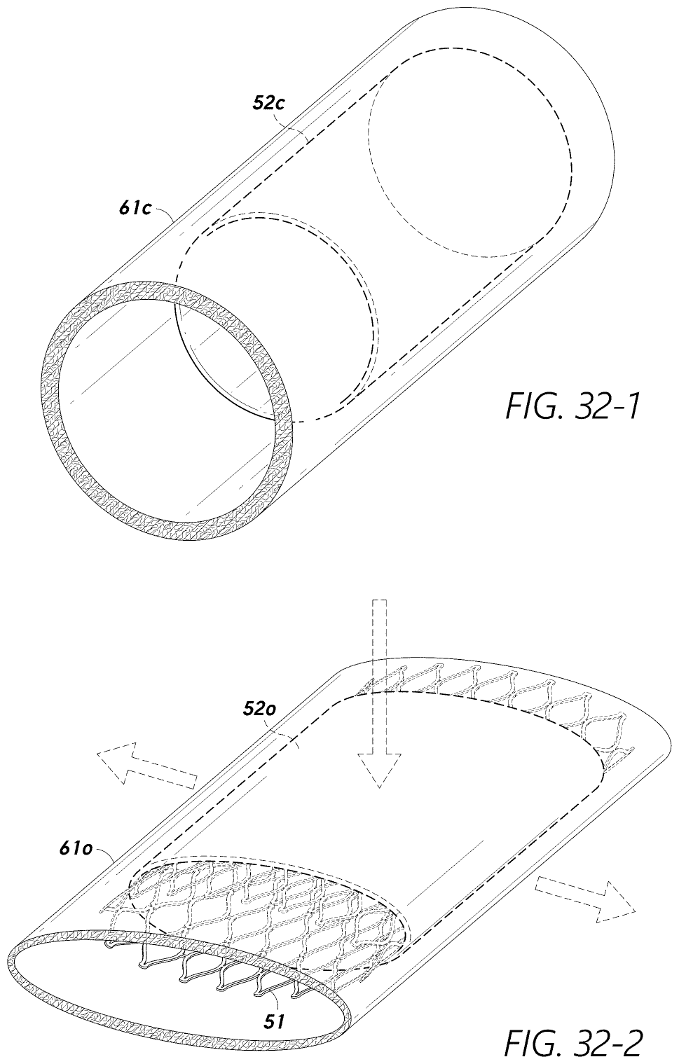

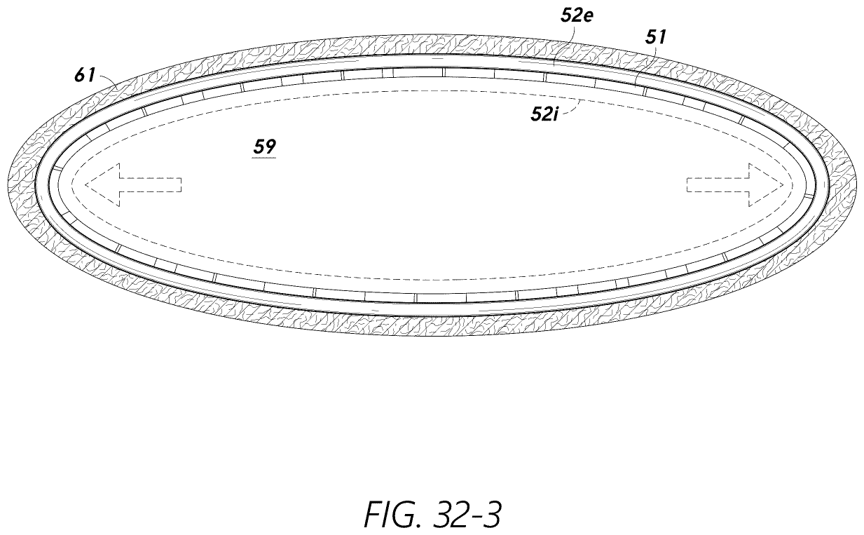

FIG. 32-1 shows a circular balloon 52 disposed within a blood vessel 61 in accordance with one or more examples. FIGS. 32-2 and 32-3 show perspective and axial views, respectively, of a non-circular stent 51 reshaping the balloon 52 and blood vessel 61 of FIG. 32-1 to a non-circular shape in accordance with one or more examples.

In FIG. 32-1, the balloon 52 is shown deployed in a target blood vessel 61, wherein the tube 52 is initially deployed and inflated as having a circular axial cross-sectional shape 520 that conforms to some degree with the natural circular shape 61c of the blood vessel 61. That is, the balloon tube 52 may have a natural circular shape 52C when inflated with inflation fluid. The balloon 52 may be inflated in a manner as to expand the tube to an outer diameter that is equal to or greater than a natural inner diameter of the blood vessel 61 to thereby secure the balloon 52 in place. Although shown as implanted separately as a separate balloon 52 in the image of FIG. 32-1, it should be understood that the balloon 52 may be implanted around (or inside) a stent 51, which is described below.

After (or concurrent with) deployment and/or expansion of the circular balloon 52c, a stent 51 may be disposed within or without the balloon 52. That is, although the stent 51 is shown within the channel of the balloon 52 in FIG. 32-2, it should be understood that the stent 51 may be outside of the balloon tube 52 in some implementations. The stent 51 may be expanded to a non-circular shape, as shown in FIG. 32-2. For example, the shape of the stent may be an oval shape as described in detail herein. Such oval expansion of the stent 51 may cause the balloon 52 to likewise assume an ovalized/non-circular shape 520, as shown. For example, the expansion of the stent 51 may cause major axis ends of the balloon 52 to be pushed radially outwardly, as identified by dashed arrows in FIG. 32-2, which may further cause minor-axis walls of the tube to be deflected radially inwardly, as shown. The stent 51 may have a biased/natural oval/non-circular shape, or a balloon or other device may be implemented to cause the expansion of the stent to reshape the stent in the non-circular/oval shape shown.

With the balloon 52 having been remodeled/reshaped to the oval/non-circular shape 520 shown, the stent 51 may be removed, thereby leaving the ovalized balloon 52o in place within the blood vessel 61 to cause reshaping thereof in accordance with aspects of the present disclosure. That is, the balloon 52 may be plastically reshaped by the stent 51 such that it holds its oval shape after removal of the stent 51. Alternatively, the stent 51 may be permanently/semi-permanently maintained within or without the balloon 52 to hold and/or otherwise control the reshaping thereof. FIG. 32-3 shows an axial view of the balloon tube 52 and stent 51 in the reshaped configuration thereof. As shown in FIG. 32-3, the balloon may be disposed radially outside of the stent 51, as represented by the depiction of the balloon 52e, and/or the balloon may be disposed within the stent 51, as represented by the dashed balloon 52i. In either implementation, the stent 51 may cause oval reshaping of the balloon 52, thereby ultimately resulting in reshaping of the blood vessel 61 to provide compliance enhancement as described herein.

### ADDITIONAL DESCRIPTION OF EXAMPLES

Provided below is a list of examples, each of which may include aspects of any of the other examples disclosed herein. Furthermore, aspects of any example described above may be implemented in any of the numbered examples provided below.

Example 1: An implant device comprising a stent frame having an oval cross-sectional shape and a fluid-tight covering disposed on a portion of the stent frame.

Example 2: The implant device of any example herein, in particular example 1, wherein the oval cross-sectional shape includes first and second arched end walls on opposite major-axis ends of the stent frame, and first and second sidewalls on opposite minor-axis ends of the stent frame.

Example 3: The implant device of any example herein, in particular example 2, wherein the first and second sidewalls are deflected radially inward to form a peanut shape of the stent frame.

Example 4: The implant device of any example herein, in particular examples 1-3, wherein the covering is on an inner diameter of the stent frame.

Example 5: The implant device of any example herein, in particular example 4, wherein the covering projects into a plane of the stent frame within cells of the stent frame.

Example 6: The implant device of any example herein, in particular examples 1-5, wherein the covering is on an outer diameter of the stent frame.

Example 7: The implant device of any example herein, in particular examples 1-6, wherein the covering is on both an inner diameter and an outer diameter of the stent frame.

Example 8: The implant device of any example herein, in particular examples 1-7, wherein the covering is disposed on minor-axis sidewall portions of the stent frame.

Example 9: The implant device of any example herein, in particular example 8, wherein the covering is not disposed on major-axis end wall portions of the stent frame.

Example 10: The implant device of any example herein, in particular examples 1-9, wherein the stent frame forms a central minor-axis diameter, outer minor-axis diameters on opposite sides of the central minor-axis diameter, the outer minor-axis diameters being greater than the central minor-axis diameter, and a major-axis diameter that is at least twice as long as the outer minor-axis diameters.

Example 11: The implant device of any example herein, in particular examples 1-10, wherein the stent frame includes one or more hinge features.

Example 12: The implant device of any example herein, in particular example 11, wherein the one or more hinge features comprises a plurality of circumferentially-offset hinge features.

Example 13: The implant device of any example herein, in particular example 11 or any example herein, in particular example 12, wherein the one or more hinge features are formed by one or more longitudinal gaps in the stent frame.

Example 14: The implant device of any example herein, in particular examples 11-13, wherein the one or more hinge features are formed by relatively thin stent frame structure providing structural circumferential discontinuity in the stent frame in positions of the one or more hinge features.

Example 15: The implant device of any example herein, in particular examples 11-14, wherein the one or more hinge features each separate a major-axis end wall of the stent frame from a minor-axis sidewall of the stent frame.

Example 16: The implant device of any example herein, in particular example 15, wherein the one or more hinge features impede transfer of mechanical force between the major-axis end wall and the minor-axis sidewall.

Example 17: The implant device of any example herein, in particular example 15 or any example herein, in particular example 16, further comprising one or more stopper extensions extending into a lumen of the stent frame from at least one of the major-axis end wall or the minor-axis sidewall.

Example 18: The implant device of any example herein, in particular example 17, wherein the one or more stopper extensions comprises a first stopper extension extending from the major-axis end wall, and a second stopper extension extending from the minor-axis sidewall, wherein the second stopper extension is configured to deflect into interference contact with the first stopper extension in response to radially-outward deflection of the minor-axis sidewall.

Example 19: The implant device of any example herein, in particular example 18, wherein the interference contact increases transfer of mechanical force between the minor-axis sidewall and the major-axis end wall.

Example 20: The implant device of any example herein, in particular examples 17-19, wherein the one or more stopper extensions extend from portions of the stent frame that are adjacent respective ones of the one or more hinge features.

Example 21: The implant device of any example herein, in particular examples 1-20, wherein the covering has one or more fenestrations therein through which fluid can flow.

Example 22: The implant device of any example herein, in particular example 21, wherein the one or more fenestrations are associated with one or more major-axis ends of the stent frame.

Example 23: The implant device of any example herein, in particular example 21 or any example herein, in particular example 22, further comprising a branch stent projecting radially outwardly from an outer diameter of the stent frame, one of the one or more fenestrations providing fluid access into the branch stent.

Example 24: The implant device of any example herein, in particular example 23, wherein the covering covers at least a portion of the branch stent.

Example 25: The implant device of any example herein, in particular examples 21-24, wherein the stent frame comprises struts forming open cells through which the fluid can flow through the one or more fenestrations.

Example 26: The implant device of any example herein, in particular examples 1-25, wherein the stent frame has one or more scallop features formed in an axial end portion thereof.

Example 27: A stent implant device comprising a first end portion having a circular cross-sectional shape, and a medial portion having a non-circular cross-sectional shape.

Example 28: The stent implant device of any example herein, in particular example 27, wherein the first end portion and the medial portion are formed from a stent frame.

Example 29: The stent implant device of any example herein, in particular example 28, further comprising a covering disposed on at least one of an inner diameter or an outer diameter of the stent frame.

Example 30: The stent implant device of any example herein, in particular examples 27-29, wherein a perimeter of the first end portion is equal to a perimeter of the medial portion.

Example 31: The stent implant device of any example herein, in particular examples 27-30, further comprising a transitional portion that transitions from the circular cross-sectional shape to the non-circular cross-sectional shape.

Example 32: The stent implant device of any example herein, in particular examples 27-31, wherein the medial portion has a peanut cross-sectional shape.

Example 33: The stent implant device of any example herein, in particular examples 27-32, wherein the medial portion spans a majority of a length of the stent implant device.

Example 34: The stent implant device of any example herein, in particular examples 27-33, further comprising a second end portion having a circular cross-sectional shape, the first and second end portions being disposed on opposite axial sides of the medial portion.

Example 35: The stent implant device of any example herein, in particular examples 27-34, wherein the medial portion is configured to reshape from the non-circular cross-sectional shape to a more-circular cross-sectional shape in response to fluid pressure within a lumen of the stent implant device that is greater than a threshold level.

Example 36: The stent implant device of any example herein, in particular example 35, wherein said reshaping of the medial portion involves expansion of a minor-axis dimension of the medial portion.

Example 37: The stent implant device of any example herein, in particular example 36, wherein said reshaping of the medial portion further involves contraction of a major-axis dimension of the medial portion.

Example 38: The stent implant device of any example herein, in particular example 37, wherein said expansion of the minor-axis dimension and said contraction of the major-axis dimension causes the major-axis dimension and the minor-axis dimension to become substantially equal.

Example 39: The stent implant device of any example herein, in particular examples 35-38, wherein, when the medial portion is in the more-circular cross-sectional shape, the medial portion combined with the first end portion to form a cylindrical form.

Example 40: The stent implant device of any example herein, in particular examples 27-39, wherein the medial portion has a major-axis dimension that is at least twice a maximum minor-axis dimension of the medial portion and the end portion has a diameter that is greater than the minor-axis dimension of the medial portion and less than the major-axis dimension of the medial portion.

Example 41: The stent implant device of any example herein, in particular examples 27-40, wherein the medial portion has an oval shape with minor-axis sidewalls that bow radially outward.

Example 42: The stent implant device of any example herein, in particular examples 27-41, wherein the medial portion comprises circumferentially-offset hinges separating minor-axis sidewall segments of the medial portion from major-axis end wall segments of the medial portion.

Example 43: The stent implant device of any example herein, in particular examples 27-42, wherein, when the stent implant device is deployed in a blood vessel lumen, the stent implant device reshapes a longitudinal segment of the blood vessel lumen into an oval shape.

Example 44: The stent implant device of any example herein, in particular examples 27-43, wherein the first end portion is configured to form a hemostatic seal between the stent implant device and a blood vessel in which the stent implant device is deployed.

Example 45: The stent implant device of any example herein, in particular examples 27-44, wherein the first end portion has one or more scallop cut-outs formed therein.

Example 46: The stent implant device of any example herein, in particular example 45, wherein the one or more scallop cut-outs comprise first and second cut-outs on opposite circumferential sides of the first end portion.

Example 47: The stent implant device of any example herein, in particular example 46. wherein the first and second cut-outs are aligned with respective major-axis ends of the medial portion.

Example 48: A stent implant device comprising first and second end portions having a first oval cross-sectional shape with outwardly-bowed minor-axis sidewalls, and a medial portion having a second oval cross-sectional shape with inwardly-deflected minor-axis sidewalls.

Example 49: The stent implant device of any example herein, in particular example 48, wherein the first end portion, the second end portion, and the medial portion are formed from a unitary stent frame structure comprising struts forming open cells.

Example 50: The stent implant device of any example herein, in particular example 49, further comprising a covering disposed on at least one of an inner diameter or an outer diameter of the stent frame structure.

Example 51: The stent implant device of any example herein, in particular examples 48-50, wherein the first end portion, the second end portion, and the medial portion have a common circumference length.

Example 52: The stent implant device of any example herein, in particular examples 48-51. further comprising a first transitional portion that transitions from the first oval cross-sectional shape to the second oval cross-sectional shape between the first end portion and the medial portion.

Example 53: The stent implant device of any example herein, in particular examples 48-52, wherein the medial portion spans a majority of a length of the stent implant device.

Example 54: The stent implant device of any example herein, in particular examples 48-53, wherein the medial portion is configured to reshape from the second oval cross-sectional shape to a more-circular cross-sectional shape in response to fluid pressure within a lumen of the stent implant device that is greater than a threshold level.

Example 55: The stent implant device of any example herein, in particular example 54, wherein said reshaping of the medial portion involves expansion of a minor axis dimension of the medial portion.

Example 56: The stent implant device of any example herein, in particular examples 48-55, wherein the first and second end portions have a major-axis dimension that is greater than a major-axis dimension of the medial portion.

Example 57: The stent implant device of any example herein, in particular examples 48-56, wherein at least one of the first or second end portions has a scallop cut-out formed therein.

Example 58: The stent implant device of any example herein, in particular example 57, wherein the scallop cut-out is aligned with a major-axis end of the medial portion.

Example 59: The stent implant device of any example herein, in particular examples 27-58, wherein the stent implant device is sterilized.

Example 60: An implant device comprising a tubular balloon having a non-circular axial cross-sectional shape, the tubular balloon forming an axial channel therethrough.

Example 61: The implant device of any example herein, in particular example 60, wherein the tubular balloon is configured to reshape a blood vessel segment in which the tubular balloon is implanted from a circular cross-sectional shape to an oval cross-sectional shape.

Example 62: The implant device of any example herein, in particular example 60, wherein the non-circular cross-sectional shape is an oval shape.

Example 63: An implant device comprising a non-circular stent comprising a stent frame, and one or more tubular balloons disposed within the stent frame and configured to be inflated to shape the stent frame.

Example 64: The implant device of any example herein, in particular example 63, wherein the one or more tubular balloons comprises a first donut-shaped balloon associated with a first end portion of the stent frame, a second donut-shaped balloon associated with a medial portion of the stent frame, and a third donut-shaped balloon associated with a second end portion of the stent frame.

Example 65: The implant device of any example herein, in particular example 63, wherein the one or more tubular balloons comprises a single balloon that spans most of a length of the stent frame.

Example 66: A method of reshaping a blood vessel, the method comprising deploying a stent within a blood vessel segment, inflating one or more tubular balloons within the stent to thereby re-shape at least a portion of the stent to a less-circular cross-sectional shape.

Example 67: The method of any example herein, in particular example 66, wherein said deploying the stent within the blood vessel segment comprises expanding the stent to a circular expanded state.

Example 68: The method of any example herein, in particular example 66, wherein said inflating the one or more tubular balloons comprises inflating a first balloon to reshape a first portion of the stent to a first shape and inflating a second balloon to reshape a second portion of the stent to a second shape.

Example 69: The method of any example herein, in particular example 66, further comprising deflating the one or more tubular balloons.

Example 70: The method of any example herein, in particular example 69, further comprising, after said deflating the one or more tubular balloons, removing the one or more tubular balloons from within the stent.

Depending on the example, certain acts, events, or functions of any of the processes or algorithms described herein can be performed in a different sequence, may be added, merged, or left out altogether. Thus, in certain examples, not all described acts or events are necessary for the practice of the processes.

Conditional language used herein, such as, among others, “can,” “could,” “might,” “may,” “e.g.,” and the like, unless specifically stated otherwise, or otherwise understood within the context as used, is intended in its ordinary sense and is generally intended to convey that certain examples include, while other examples do not include, certain features, elements and/or steps. Thus, such conditional language is not generally intended to imply that features, elements and/or steps are in any way required for one or more examples or that one or more examples necessarily include logic for deciding, with or without author input or prompting, whether these features, elements and/or steps are included or are to be performed in any particular example. The terms “comprising,” “including,” “having,” and the like are synonymous, are used in their ordinary sense, and are used inclusively, in an open-ended fashion, and do not exclude additional elements, features, acts, operations, and so forth. Also, the term “or” is used in its inclusive sense (and not in its exclusive sense) so that when used, for example, to connect a list of elements, the term “or” means one, some, or all of the elements in the list. Conjunctive language such as the phrase “at least one of X, Y and Z,” unless specifically stated otherwise, is understood with the context as used in general to convey that an item, term, element, etc. may be either X, Y or Z. Thus, such conjunctive language is not generally intended to imply that certain examples require at least one of X, at least one of Y and at least one of Z to each be present.

It should be appreciated that in the above description of examples, various features are sometimes grouped together in a single example, Figure, or description thereof for the purpose of streamlining the disclosure and aiding in the understanding of one or more of the various inventive aspects. This method of disclosure, however, is not to be interpreted as reflecting an intention that any claim require more features than are expressly recited in that claim. Moreover, any components, features, or steps illustrated and/or described in a particular example herein can be applied to or used with any other example(s). Further, no component, feature, step, or group of components, features, or steps are necessary or indispensable for each example. Thus, it is intended that the scope of the inventions herein disclosed and claimed below should not be limited by the particular examples described above, but should be determined only by a fair reading of the claims that follow.

It should be understood that certain ordinal terms (e.g., “first” or “second”) may be provided for ease of reference and do not necessarily imply physical characteristics or ordering. Therefore, as used herein, an ordinal term (e.g., “first,” “second,” “third,” etc.) used to modify an element, such as a structure, a component, an operation, etc., does not necessarily indicate priority or order of the element with respect to any other element, but rather may generally distinguish the element from another element having a similar or identical name (but for use of the ordinal term). In addition, as used herein, indefinite articles (“a” and “an”) may indicate “one or more” rather than “one.” Further, an operation performed “based on” a condition or event may also be performed based on one or more other conditions or events not explicitly recited.

Unless otherwise defined, all terms (including technical and scientific terms) used herein have the same meaning as commonly understood by one of ordinary skill in the art to which example examples belong. It be further understood that terms, such as those defined in commonly used dictionaries, should be interpreted as having a meaning that is consistent with their meaning in the context of the relevant art and not be interpreted in an idealized or overly formal sense unless expressly so defined herein.

The spatially relative terms “outer,” “inner,” “upper,” “lower,” “below,” “above,” “vertical,” “horizontal,” and similar terms, may be used herein for ease of description to describe the relations between one element or component and another element or component as illustrated in the drawings. It be understood that the spatially relative terms are intended to encompass different orientations of the device in use or operation, in addition to the orientation depicted in the drawings. For example, in the case where a device shown in the drawing is turned over, the device positioned “below” or “beneath” another device may be placed “above” another device. Accordingly, the illustrative term “below” may include both the lower and upper positions. The device may also be oriented in the other direction. and thus the spatially relative terms may be interpreted differently depending on the orientations.

Unless otherwise expressly stated, comparative and/or quantitative terms, such as “less,” “more,” “greater,” and the like, are intended to encompass the concepts of equality For example, “less” can mean not only “less” in the strictest mathematical sense, but also, “less than or equal to.”

What is claimed is:

1. An implant device comprising:
a unitary stent frame including:
a first axial end segment having a circular cross-sectional shape;
a second axial end segment having a circular cross-sectional shape; and
a medial segment formed of shape memory metal disposed between the first axial end segment and the second axial end segment, the medial segment having an oval cross-sectional shape; and
a fluid-tight covering comprising ultra-high molecular weight polyethylene and disposed on minor-axis sidewall portions of the medial segment of the unitary stent frame.

2. The implant device of claim 1, wherein the unitary stent frame is sized for implantation in a pulmonary artery for enhancing compliance of the pulmonary artery.

3. The implant device of claim 1, wherein:
the oval cross-sectional shape of the medial segment includes:
first and second arched end walls on opposite major-axis ends of the unitary stent frame; and
first and second sidewalls on opposite minor-axis ends of the unitary stent frame; and
the first and second sidewalls are deflected radially inward to form a peanut cross-sectional shape of the unitary stent frame.

4. The implant device of claim 1, wherein:
the fluid-tight covering is not disposed on major-axis end wall portions of the unitary stent frame.

5. The implant device of claim 1, wherein the unitary stent frame forms:
a central minor-axis diameter;
outer minor-axis diameters on opposite sides of the central minor-axis diameter, the outer minor-axis diameters being greater than the central minor-axis diameter; and
a major-axis diameter that is at least twice as long as the outer minor-axis diameters.

6. The implant device of claim 1, wherein the medial segment of the unitary stent frame includes a plurality of circumferentially-offset hinge features formed by:
one or more longitudinal gaps in the unitary stent frame; or
relatively thin stent frame structure providing structural circumferential discontinuity in the unitary stent frame in positions of the plurality of circumferentially-offset hinge features.

7. The implant device of claim 6, wherein the plurality of circumferentially-offset hinge features each separate a major-axis end wall of the unitary stent frame from a minor-axis sidewall of the unitary stent frame to impede transfer of mechanical force between the major-axis end wall and the minor-axis sidewall.

8. The implant device of claim 1, wherein:
the fluid-tight covering has one or more fenestrations therein through which fluid can flow; and
the one or more fenestrations are associated with one or more major-axis ends of the unitary stent frame.

9. The implant device of claim 1, wherein the unitary stent frame has one or more scallop features formed in an axial end portion thereof.

10. A stent implant device comprising:
a first and second axial end portions having a circular cross-sectional shape; and
a medial portion disposed between the first and second axial end portions, the medial portion having a biased peanut cross-sectional shape that has a major-axis diameter that is greater than a diameter of the circular cross-sectional shape of the first and second axial end portions;
wherein the medial portion is adapted to:
transition from the peanut cross-sectional shape to an oval cross-sectional shape under baseline blood pressure conditions; and
transition from the oval cross-sectional shape to the circular cross-sectional shape under systolic blood pressure conditions when a major-axis dimension of the medial portion is compressed and minor-axis sidewalls of the medial portion are deflected radially outward.

11. The stent implant device of claim 10, wherein the first and second axial end portions and the medial portion are formed from a unitary stent frame, the stent implant device further comprising a covering disposed on at least one of an inner diameter or an outer diameter of the unitary stent frame.

12. The stent implant device of claim 11, further comprising:
a first covering disposed along an inner diameter of the unitary stent frame; and
a second covering disposed along an outer diameter of the unitary stent frame to sandwich the unitary stent frame between the first covering and the second covering.

13. The stent implant device of claim 10, wherein a first perimeter of the first axial end portion is equal to a second perimeter of the medial portion.

14. The stent implant device of claim 10, wherein:
the major-axis dimension of the medial portion is at least twice a maximum minor-axis dimension of the medial portion when the medial portion has the biased peanut cross-sectional shape; and
the first and second axial end portions have a diameter that is greater than the maximum minor-axis dimension of the medial portion and less than the major-axis dimension of the medial portion.

15. The stent implant device of claim 10, wherein:
the first axial end portion has one or more scallop cut-outs formed therein;
the one or more scallop cut-outs comprise first and second cut-outs on opposite circumferential sides of the first axial end portion; and
the first and second cut-outs are aligned with the major-axis dimension of the medial portion.

16. A stent implant device comprising:
a first axial end segment having a first oval cross-sectional shape with outwardly-bowed minor-axis sidewalls;
a second axial end segment having the first oval cross-sectional shape with outwardly-bowed minor-axis sidewalls; and
a medial segment disposed between the first axial end segment and the second axial end segment, the medial segment having a peanut cross-sectional shape with inwardly-deflected minor-axis sidewalls.

17. The stent implant device of claim 16, wherein:
the first axial end segment, the second axial end segment, and the medial segment are formed from a unitary stent frame structure comprising memory metal struts forming open cells; and
the stent implant device further comprises an ultra-high molecular weight polyethylene covering disposed on at least one of an inner diameter or an outer diameter of the unitary stent frame structure and covering at least some of the open cells.

18. The stent implant device of claim 16, wherein the medial segment spans a majority of a length of the stent implant device.

19. The stent implant device of claim 16, wherein the medial segment is configured to reshape from the peanut cross-sectional shape to a second oval cross-sectional shape in response to fluid pressure within a lumen of the stent implant device that is greater than a first threshold level.

20. The stent implant device of claim 19, wherein:
the medial segment is configured to reshape from the second oval cross-sectional shape to a more-circular cross-sectional shape in response to fluid pressure within the lumen of the stent implant device that is greater than a second threshold level that is higher than the first threshold level; and
said reshaping of the medial segment involves expansion of a minor axis dimension of the medial segment.
